SEQUENCE LISTING (57 sequences)

SEQ ID NO: 1 - DNA, 16 bp, Artificial Sequence
  Iteron
tgacacttgaggggca

SEQ ID NO: 2 - DNA, 16 bp, Artificial Sequence
  Iteron
tgacacttgaggggcg

SEQ ID NO: 3 - DNA, 16 bp, Artificial Sequence
  Iteron
tgacatttgaggggcc

SEQ ID NO: 4 - DNA, 16 bp, Artificial Sequence
  Iteron
tgacacttgaggggcg

SEQ ID NO: 5 - DNA, 4841 bp, Artificial Sequence
  anti-F cassette
caacacacaccagacaagagagctgcgtggtagtttcatggccttcttctccttgcgcaaagcgcggtaagaggctatcctgatgttgtctaagcatgcaggggcctcgtgggttaatgaaaaattaactacggggcttttgtccttctgccacacaacacggtaacaaaccaccttcacgtcatgaggcagaaagcctcaagcgccgggcacatcatagcccatataccagcacgctgaccacattcacttttcctaagcttacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatgccttgagcaactgatagctgtcgctgtcaactgtcactgtaatacgctgcttcatagcacacctctttttgacatacttcgggtatacatatcagtatatattcttatgccgcaaaaatcagcgcgcaaatacgcatactattatctggcttttagtaagccttatgtattttacctttcgttatgttaaccaataaaaattaaaatctgccttataaaaacaaagcgtaattaccgcattcccgtttcgtatggtctagcaccacgctgggtttactgtttggttgaaagttatatttttattaaacattgtgcgttaaagcctggtgtgtttttttagtggatgttatatttaaatataacttttatggaggtgaagaatgcataccacccgactgaagagggttggcggctcagttatgctgaccgtcccaccggcactgctgaatgcgctgtctctgggcacagataatgaagttggcatggtcattgataatggccggctgattgttgagccgtacagacgcccgcaatattcactggctgagctactggcacagtgtgatccgaatgctgaaatatcagctgaagaacgagaatggctggatgcaccggcgactggtcaggaggaaatctgacatggaaagaggggaaatctggcttgtctcgctggatcgggtacctctcgcacagcgattttcgtgtcagataagtgaatatcaacagtgtgagacacacgatcaacacacaccagacaagggaacttcgtggtagtttcatggccttcttctccttgcgcaaagcgcggtaagaggctatcctgatgtggactagacatagggatgcctcgtggtggttaatgaaaattaacttactacggggctatcttctttctgccacacaacacggcaacaaaccaccttcacgtcatgaggcagaaagcctcaagcggctagaggaggctcgatccagtaaacagatccatgaatgatcaacaaaggatccattaaagatccccataccgctgcaaaccttgtcactcatgggccgggaccacgatcacataagcagtggcatgttactgataaactgtaacatgctaatgataagctgtattcagtaatccatatactgaagtaagttaatgacataaactatggtcagtacgccagactcagctgttaaatacaggctgcaggtttttcttcagtcagttagcggggctctgacacacgatttgctgtttattcttttactgtccacaggcaggaggctttctggaaaacgaaaattcagacatcaaaaaactgttcggcgaggtggataagtcgtccggtgagctggtgacactgacaccaaacaataacaacaccgtacaacctgtggcgctgatgcgtctgggcgtttttgtaccgacccttaaatcactgaagaacagtaaaaaaaatacactgtcacgtactgatgccacggaagagctgacacgtctttccctggcccgtgctgagggattcgataaggttgagatcaccggcccccgcctggatatggataacgatttcaagacctgggtggggatcattcattcctttgcccgccataacgtaattggtgacaaagttgaactgccttttgtcgagtttgcaaaactgtgtggtataccttcaagccagtcatcacgcaggctgcgtgagcgcatcagcccttccctgaaacgcattgccggtaccgtgatctccttttcccgcaccgatgagaagcacacccgggaatacatcacccatctggtacagtcagcctactacgatactgaacgggatattgttcagttacaggctgatccccgcctttttgaactgtaccagtttgacagaaaggtccttctccagcttaaggcgattaatgccctgaagcgacgggagtccgcccaggcactctacacctttatagagagcctgccccgggatccggcaccggtatcgctggcgcggctgcgtgcacgcctcaatctgaagtctcctgtattttcccagaaccagacggtcagacgggcaatggagcagctgcgcgagattggatatcttgattacacggagatccagcgggggcggacaaaactcttctgcattcactaccggcgtccccggttaaaagcaccgaatgatgagagtaaggaaaatccgttgccaccttcacctgcggaaaaagtcagtccggagatggcggagaagcttgccctgcttgagaaactgggcatcacgctggatgacctggaaaaactcttcaaatcccgctgaacataaactgtagtcagtgaagagtgttcctttactgactacagcttatattatcaggtgcagtgagtggtctgctcactgcagtttatattcagtttcctgcagtgctgcctgtagctgagctgtcatctgccggtcccttacgtgagtcaccccgtaacctgatgctgaggcattgctcccttcataaaacatgacttactcactacagcttatatacatgctccagcttatgttatgtctgttctgctgaccacagcttgtcgagcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggatcccctgtcggtaatgagtgaacagtgttcgggtgtcagtcacttctgcctgcacttacaatgcacggaaggagtaaatgccacagaaacaagtattgaaattttctgagcataaaactatactccctgcatagcctgattggtggctatacagtttaagtaggccccgttaatctttcgttcccgccaagaaatgagaagattatcggggtttttgcttttctggtcccgggtaggcacctcatcagaaccagttccctgccaccttacggcgtggccagccatcaaaattctttagacgatcagcaatctaacactcactgacgagacgatcaagaaagttactcattcaccttcttatctcaggctcttttagccatttcccgttcaattagtagctcaatcatttgagcctgagtagttccagtctcttcacaaagctgctgcaagctttctttatgtatgttttgtatgaaaacattgagctctttatgcgtcgtcttttttcgtgaaatagataatctcttcttctcagcggaagataagggattaccctttctgtaaatgcgtttcgatgaggaagttactgcattttcaatctgcgacatctctgtctcctcaaggatcaaacctagatcgtttgcagagaatactacagatttctaaaatcagcgacttactttatgaacgtaacctgtgttggcgcacggtttacgtttcaagatcgtttcctgggatgtcagtacgtctgtttccagtccgttccctcgactgaagatcagtcacaccatcctgcacttacaatgcgcagaaggagcgagcacagaaagaagtcttgaacttttccgagcatataactatactccccgcatagctgaattgttggctatacggtttaagtgggccccggtaatcttctcagtcgccaaactttctgaagattatcggggtttttgcttttctggctcctgtaaatccacatcagaaccagttccctgccaccttacggcgtggccagccacaaaattccttaaacgatcagtaatctagcactaatcttctgaacactcaagaatgtaagcccatcatcacacacatcgtttttgcgcttcactttttatcagtgcggtcagaacttcagcctgagtcaggccatcttcatgacacatttgcatgagcatggccttatactttggttcaagaaatacttttacttccttgaacgaagctcttttacgggccactgataatctttgtttctctgcatcagaaagcggattcccctttctgtatgctcgttttgcgccagatgaggaagtcactgcattttctgtctgcgacatctcgcctcctcaatacttaaacagggatcgtttcgcagaggatactacagttttttgaaatcagcgacttgagaattgtgacgaagatccgggattaccctgttatccc

SEQ ID NO: 6 - DNA, 133 bp, Artificial Sequence
  anti-I cassette
tgttccggaagccataaaaggaaaacccccactatctttcttacgaacttggcggaacgacgaaagatagtgggggcctcacagaatacgggtaaagtataatgaaaccgtaccagagattcaaccctgtgca

SEQ ID NO: 7 - DNA, 541 bp, Artificial Sequence
  anti-K cassette
ccatggccataaggcattcaggacgtatggcagaaacgacggcagtttgccggtgccggaaggctgaaaaaagtttcagaaggccataaaggaaaacccccactatctttcctcgaactttggcgggctcgtgaaagatagtaggggcgttcacagaatacgggataagtatatatgaaaccgtaccagagattcaaccctgtgcagtgtataaatacacggcacaatcgctccgccataagcgacagcttgtggcaggtctgaagaatacttcatataacgcagtacactggagtcagttagcacccgaagagcagatccgtttctgggaagactatgaagcgggaagggcgaccactttcctggttgaaccggaaaggaagcgcacgaagcgccgtcgcggtgagcactccaccaaacccaaatgcgaaaatccgtcctggtatcgtccggagcgctataaggcgctgagcgggcagctcgggcacgcctacaaccgtctggtgaaaaaggacccggtgaccggcgagcagagcctgcg

SEQ ID NO: 8 - DNA, 19 bp, Artificial Sequence
  consensus sequence
  misc_feature (8)..(8): W
  misc_feature (9)..(9): r
tgagggtnntttgtcacag

SEQ ID NO: 9 - DNA, 57415 bp, Artificial Sequence
  plasmid
ttcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgccgcgatgaggaagcgggtgccccgcttcttcatcttcgcgcctcgggcctcgaggccgcctacctgggcgaaaacatcggtgtttgtggcattcatacggactcctgttgggccagctcgcgcacgggctggcgggtcagcttggcttgaagatcgccacgcattgcggcgatctgcttctcggcatccttgcgcttctgcacgccttcctgctggatgcgaataacgtcctcgacggtcttgatgagcgtcgtctgaacctgcttgagcgtgtccacgtcgatcaccaggcgttggttctccttcgccgtctcgacggacgtgcgatgcagcagggccgcattgcgcttcatcaggtcgttggtggtgtcgtcgatggccgtggccagttcgacggcgttcttctgctcgttgaggctcaaggccagcatgaattgccgcttccacgccggcacggtgatttcgcggatggtgtggaatttatcgaccagcatctggttgttggcctggatcatgcggatggtcggcaggctctgcatggccgaatgttgcaaggcgatcaggtcgccgatgcgcttgtccaggttggcaaccatcgcatcgaggtcggccagctcctgcacgcggcccgggtcgttcccgacattgccgcgcagaccctcggcctgctcgcgcagctcggcaaggcggaccttgccggccgcgatgtggacgccaagaaggcggtgttcctcgcgcacggctgcgaacatttcgtcgagcgaggcattgcgctgcgcgatgccttgctgggtggtctgcacttcgctgaccaggtgttcgatctgctcgcgggtcgtgtcgaagcgcgccatgaagcccgtcgaacggacgcggaagcggtcgatcagcgggccaatcaggggcaggcgggaacggttgtcggacaaagggccgacgttcagggaacgggccttggcgacaacctgggtcagtttctcgcctgcttcgtccaggtcgctgttgcgcacctggtccagcaggctatcggcgtagcgggacgtgtgctcggccacgtcgcggccgaactcggcaacggtctgcggactgccgacctcgatccgctgcgcgaccgcatggacttccggcacgtcgctttcctgcaagcccagctcgcgcagggttgccggggtcatgtcgaaggcgacgataggggccttggcgtcgtgcgtcgttttcagtgcgttcatagggttctcccgccgtgttattggttgatgccttccaggctctgcgaaaggctccgcatgagcgcctggtgagctttggccgcctcggcgaccattgccggattcatgttcttggtggtgatgagcgcgagggtgtgctgacgccagacgggcaccaggacggatgccgtttcagagaagcggtccagcatgtccacggcctgcgcccgcgtgagcttcatctgagtgacgctcatttcatgggacgccatgagggttgccaggttggcgagcttgcgcgcgaagcgttcgcgcggcttgtcgaactcgatcacgccggccttggccgcgccggcctcggggttctcgtccaggaactcgcgcccggcttgaatgtaggctctgagccggtctacctcggcctcatgcgtattgagcatgtcatccaaggcgcgcaacgtgtcccgcacgcgctgcgctacgccctcggcttcgtccagcaactggtcgagcgtcttgcgggcgacctgatacctcacctggcgttcaacctcacggccaagcatcttctcgaaccaggtaggcttttccgcgatcttgcgggggtccgcgtcggccagcttcgccacgatctggctgattttgtcggccagcgcggcaactgcgccgtgctccatcagattcgacagctcgttgagggaatccgccccgtcgatgccggccccgtactcgccaatcgtcgccggcgacgcgaagagggcgggcaaaacctcccccttcaatcgcgccatgttcacgctttgttcttccattcgatacaccctcgcggtgggttaattgcttttcgatggaagaagtttagctaaactttctatccctcgtcaacacctttagccgctaaaatttggggacaggtcatttacagaaagccagctcactcctggcgttgccccttgagcgccgctaggcgcgcagcatccttcgcgctgagaaagaacgtcatcagcggcccgaccgtcttgcttgaaccgtcggcaaagcaaacatccatcgaacagccttgcgtgtgggggtccacgccttcgaccagtttccaagggtccatgccccagccctcggcctccggattgaaccagtacgccgatgcgtcgccgtttaggtcgctgtcggcgtagtccttgaccagcacggccacccgcccggtcgtcgggcacacgtagcccggctgcttaggttcctgtcttggcattgctcaaagctccttgaaggggccgctctacagccccttgggcttgtagagcgacacgaaataggtgagtgcggtcagtaccgcgaaatgcaccaggaacgtccagccggcatgaacgccaagggtgttccagtggtacagcatccgcaggaactgaaagaaaacgtcgatagagatgatccacttcgccaccggccacaccaggacagtaacgacccatacaaagcggaccagggcctggacacccttggcaaaagtgaaccggggcggggccttgctcggggcctcaacgcgcggggcaggggcctcggcctccacttccacgcctgggaacttgataatcttcgacattgcttgaccctccacggcgatgcgtgttcaattcgtccagcgctcgcgcgcctagaccgtgatgtgacagcatcgaggtcaagcgccccggagaaatccggggcgtcatccctatgccccgtccaactcgggaaccggcttttccctggtgctcaacctggccggctcgacccacttggtgacttgctgccactcgttaccactgcgaacggctacccgaatctgcacacgtttagccgcgatcttcgtcactatgccagcaacacaaacggaatacccgtacccgccgcgcggggtgtgctgccagttcaccctatctcctacttgcatcatcatcccctggcgtcagtgaccggcccggaatttcgccaagtcgatttcgttgaaggtccagcgctgtttcgcccgttcctccaacctcgacgactcccgcatgacctcgatgcgcaggcgctcgacctggtgcatcagctcatcagcgcgccgctgcttctcggcaatagcattccgctgcgcgaccagctcctggtctacgttcggcagctcgtcgatccacggcatgaacttatcggtcatcggattggcctccggtaattgacctgggaatctacccggcctcaaaacaagaatagggcataatgccctaacttgtcaagcaattttagctaaacaattgaggggattcagcgaggcgtcatgcttgaaaacacctttcccctggcgtgcaatcagcttgtcccggcggcagcgcactgccgcagcgcggccagcaaggtgccttgctcgatccgctgtcgttcctccggcgtaatgcaatcgccgcagataccgcccagctcttcgcgcagggcgtccgccccgatcaggctgcggctaagggtcgaaatggacttaccgcagcggcggcaattggtggtgacaatctcgatcttgcggttgggcatggccctatctccttgagagaggcccgaccgtagccgggcctcgttccgttaccagcctgcgcgagccttcgcgcgttcgacttcctgggtggaccagtggccctttgcttcaccctccagggtgcaaccttgcaggtacgccttgtgccgcagctcttcggcctggcgggtaagctctgccgcctgctccatcagcgccgcgatctggtcacgacgggccagaaaatccgtggcggccgcgccgggcagctcatcgagccaagacatgatcttgcttttcatcggggttgatcctccggttgctgacctgggcgaagtgcccggccttggattgctatattagggcattatgccctagatagtcaaggaaatttagctaaacaatttgcggcgggcgcacgaaaaaacccggcttgcgggccgggctgctggcagcatatcgcaacgatcagggttggcggttttagccgctaaagtcctctcccttggcgtaaagtcctgcgggcgtcagccctggcctttccagatcgccccaatccccgctagatcgcaaaggatcgcccaggcggcataggggatcggcgaatcctcgccaacccaacgccgcaccgtgcggtcgcccttggcacccaagcccaagatgcgcgcagcctgtccgccggtgaggccggccaagtgcaagacttcccggatttctgcgccggtcggctgcacccagcgttccgccgggcgcaggcactcaagccggatattcacgtcgctcatgctgcttttctcctaatcgttatcaaatggcggcccgcaattggtcgtagccgtagcacgactcgatgcaacgcgggtcatattcacaaacctttctaccgttgcgattgatgcgatagcgcggcggcttgtcgtcgtgcgcccacacctgtcgcgtttcgggactgatcttcgtaatgatgacgtgcttcccgatttccttggcaaaaaccgggtgatgcacgctcacaatctcgcaccgcaggccaggaaagaacgtcgccggccacgccggcgcgtcctcgtccttggccggctccggctccggcttggccggtgtaaccggctccctgcgctgcccggccggctcctgagctttcgcacgcgcggccgcgagcttcgccttggcgctggccgcccatttgcgggcgatgaatgcctggtgcgccggcagcacggcatcaacccaggcgaagcccggcggcagttcgtccggcttccgccagtcctcgaccacggcccgcacgcggcgcggcggtggcgtgatacccgcgagccacgccggcagtggctcgtcggcaaggcggtcctggtcgcgggcgtccagataccggcgatgcgagcgcaacagggcgcgcaaaaccccgtccgctacctggtccaccgtcatgccgccgcgcgcatggtcgtaatgggagacgtaattgagcatttccaggcgcttgcgcctggtcaacgaaagagtcagcgccgtaggcgctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcatcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgtggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttccaacgcgagccattcgcgagagccttgagtccacgctagatctatctcatctgcgcaaggcagaacgtgaagacggccgccctggacctcgcccgcgagcgccaggcgcacgaggccggcgcgcggacccgcgccacggcccacgagcggacgccgcagcaggagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttccttgtaggcatcgggattggcggatcaaacactaaagctactggaacgagcagaagtcctccggccgccagttgccaggccgtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgacccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccactcttcattaaccgctatatcgaaaattgcttgcggcttgttagaattgccatgacgtacctcggtgtcacgggtaagattaccgataaactggaactgattatggctcatatcgaaagtctccttgagaaaggagactctagtttagctaaacattggttccgctgtcaagaactttagcggctaaaattttgcgggccgcgaccaaaggtgcgaggggcggcttccgctgtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacgccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgccggcggcatttcgagagcctgggaacgatgtacgagggctatttcgtcctcaccttgacctggttcccgccgctgctcgcccagcgcaagttcgtcgagctgatgtttgacgacgacgcgaccgcaccggatcgcaaggcgcgcacgcggggcctcatcgaccaattcaagcgtgacgtgcgcagcatcgagtcgcgcctgtcgtcggccgtgtcgctcactcgcttgaaggggcacaagatcgtcaacgaggacggcacgaccgtcacgcatgacgacttcctgcgctggctgcaattctgcgtgacgggcctgcaccatccggtgcagctccccagcaacccgatgtacctggacgccctggtcggcggacaggaaatgtggggcggggtagtgcccaaggtcggccgcaagttcgtccaggtggtcgctctcgaaggcttccccttggagtcctatcccggcatcctgacggcgctcggcgagctgccctgcgagtatcggtggtcgagccggttcatcttcatggaccagcacgaagccgtgaagcacctcgacaagttccgcaagaagtggcggcagaagattcgcggcttcttcgaccaggtgttcaacacgaacaccggcccggtcgatcaggacgcgctttcgatggtggccgatgctgaggcggccattgccgaagtcaacagcggcatcgtggccgtgggctactacaccagcgtcgtcgtgctgatggatgaggaccgcacgcgcctggaagctgcggcccgcgatgttgaaaaggccgtcaaccggttgggctttgccgcgcgcatcgagtccatcaacaccctggacgccttccttggtagtttgccgggccacggcgtggaaaacgtccgccggccgctcatcaacacgatgaacctggccgacctgctgccgaccagcaccatctggaccggcaacgcgaacgcgccatgcccgatgtacccgccgctgtcgccggcgctcatgcactgcgtcacgcaaggatcaacgccgttccggctgaacctgcacgtgcgcgacctcggccacacctttatgttcgggccgaccggcgcaggtaaatcgacgcacctggcgatcctcgccgcgcagctccgtcgctatgccggcatgtcgatcttcgcctttgacaagggcatgtcgatgtacccgctggccgccggcatccgtgcggccacgaagggcaccagcggcctgcacttcaccgtggcggccgacgacgaacgcctggcgttctgcccgttgcagttcctgagcaccaagggcgaccgtgcttgggcgatggagtggatcgacaccatcctggcgttgaacggcgtcgaaacgaccccggcccagcgcaacgaaatcggcaacgcgatcatgagcatgcacgccagcggcgcgcgcacgctctccgagttcagcgtgacgattcaggatgaggcgatccgcgaggcgatccgccagtacaccgtcgatggcgcaatgggccatctgctcgacgccgaagaggacggcttggcgctgtccgactttacagtgttcgagatcgaagagctgatgaacctcggcgagaaattcgccctgcctgtgttgctctacctgttccgccgtatcgagcgcgccctgacgggccagccggccgtcatcatcctggacgaagcctggttgatgctcggccacccggcattccgcgcgaagatcagggaatggctcaaggtgctgcgtaaggccaactgccttgtgctgatggcaacgcagagcctgtccgacgccgccaacagcggcatcctggacgtgatcgtggaatcgaccgcgaccaagattttcctgccgaatatttacgccagggatgaggacacggcggccctgtaccgccgcatgggcctgaacgctcgccagatcgagattctggcccaggccgttcccaagcgtcagtactactacgtgtcggaaaacggccgccgtctctacgacctggcacttggcccgctcgcgctcgcgttcgtcggcgcatccgacaaggaatccgtcgccatcatcaagaacctggaagccaagttcggcgaccagtgggtggatgaatggctgcgtggccggggcctcgcccttgatgaatacctggaggcagcatgagttttgcagacacgatcaagggcttgatcttcaagaagaagcccgcaacggccgcagcagcggcgacgccggccgcgaccggcccgcaaaccgacaacccgtacctgacggcgcggcgcacctggaacgaccacgttggttccgttgtgtcgcaaaagcagacctggcaggttgtcggcatcctttcgctgatgatcgtcctcgcggcggtcggcggcatcatccacatcggcagccagtcgaagttcgtgccctatgtctacgaggtagacaagctcgggcagacggccgccgtggggccgatgaccagggcgtcgaaagccgatccgcgtgtcattcacgcctcggtggctgagttcgtcggcgatgctcgcctggtgacgccggacgtagctttgcagcgcaaggccgtctaccgcctctatgccaagctcgggccgaatgacccggccaccgccaagatgaacgaatggctcaacggcaccgccgacgccagcccgttcgctcgcgcggccgtcgaaacggtcagcaccgaaatcacttccgtaatcccgcagacgcccgacacctggcaggtcgattgggtcgagacgacgcgcgacaggcaaggcgtggtgaaaggccagcccgtgcgcatgcgggccttggtgacggtctacgtcgtcgagccgacggcggacaccaaggaagaacaactgcgaaacaacccggccgggatctacgtccgggacttctcctggtcgagacttctgtgaggcactgaattatgaaaaaggaactgtttgctttggtcctggccgcgtccgttagcgtgcctgcatttgccgccgatcccggcgcggacctgactgacctctatttttccggcaagaacccggagctgaccgcgcaagagcgggcggccatcgccatcgccaagaagtgggaggcgggtaccgccggcatgcggccggtggccggccccggtggttcggtgcgcttcctgttcggcgcgcagcagccgagcatcgtatgcgccgtgctgcaagtgtgcgacgtggccctgcaacccggcgagcaagtcaactcgatcaacctgggcgacaccgcccgttggacggtcgagccggccattaccggcagcggcgcgaacgaaacccagcacctcatcatcaagccgatggatgtgggcctggaaaccagcctggtcgtgaccacggaccgccgcagctaccacatgcgcctgcgctcgcatcgcacgcagtacatgccgcaggtgtcgttcacctacccggaagatgcccttgcgaagtgggacgccatcaagaaccgcgaacagcgggatcgcgtcgagaaaaccattccgcagaccggcgagtacctgggcaacctgagcttcaactactccgtcagcgggtccacgtcgtggaagccggtgcgcgtctacaacgacggcaagaaaaccatcatccagatgccgcactcgatggaacagaccgaagcgccgacgctcctggtcgttcgcagggagggcggcctgttctccgacgatgaaacggtgatggtcaactaccgggtccagggcgaccgctacatcgtcgatacgattttcgacaaggccatcctcatcgcgggcgtgggcagcagccaggaccgcgtgaccatttcaagggggaactaaaccatgcgtaagattctgaccgtcatcgcactcgcggccacgttggccggctgcgcgacctccaagtacggcagcttcgtccaggacgcgccggccgcctacaaccagaccattgcgaccgacgcggtgaagcagctcgtcaagctctacccgccggcgcaaaccaagctggaattgcagcaggctacgcccgatccgttcggcattgccctggtcactgaccttcgcgcccagggctatgctgtcatggagtacaagcccgacggcaacgcggccgcagctccggctgctgcgtcctcggccgctgcgaagccggcaacgccgcaagcccagggcggctatccgctgcgctacgtgctggaccaattcagcgacagcaacctgtatcgcctgaccgtcatggtcggctctcaatcgctcacgcgcgcctacctcgcccaaaacaacacgatggtcccggccggcgcatgggttcggaaggagtaagccaatgagcgaagatcaaatggcaccggacgcatcgccagatgcggtcaagccgaaaagcggggttcgccgcgtcaacaacatgccgatgtacctcatcggcggtgtgctcggcatcttcctgctggtgatggccctggttgctgcggatcgcgctgcgcagcagaaccagccgggagctgcgaaggctgagaaggccggcagcaccagcatgtttgccgacgaaattgccggcaaacagcaggacggcatcatcaaggccaagccgctggagattccgccggaacaaaccgcccagcaaccgacgacggagctgacgccagccccggcgcagggaacgactatcacggtcgcacggcccgagaacctggaccagcccccgacgccgccgcagggtgcgcgcaacgaggacctggaccgcatccgcatggcgaagttgcagatgctggaagaggcgatcaaggccaagacgacggtgcgcatcgacgcgccgcgcagccagggcagcgccggcggcggtgctccgcagggccgcgaggaaacccttgcgcgcatccaggagctgcgtcggcaggctgagaacgcccgcgccaccgatccgaccgccgcctatcaggccgcgcttgcgcaggctcgcacgatgggcggcgcggcagggggtggcggtatgggcggctcgggtgcgccgaccctcgtgcagacctcgaaccgcagtggtggcggcgctggctatgggtcgttcgacaaccgcagcgagggcgaccgttggcggctcgactcccagccggaagcacctgcaacgccctatgtgctgcgcgctggcttcgtcgttccggctacgcttatctcgggcatcaactccgatctgccaggccaaatcatggcccaggtatcgcagtcggtgtacgacacggcgaccggcaagcacatgctcatcccccaaggctcgcgcctggtgggcagctactcgaacgatgtggcctacgggcagaagcgcgttctggtggcatggcagcgcatcatcttccccgacggcaaggcaatggacattggggccatgccgggcggcgatagcgctgggtatgcaggcttcaacgacaaggtcaacaaccactacttccgcaccttcgcatcggcattcctcatgtcgggcgtcgttgcgggcatcagcttgagtcaggaccgtggcaacagcaacagcggttacggacgacaagacgcgggttccgcgatgagtgaagcgttgggtcaacagctcggccaagtaacggcgcagatgatcgccaaaaacttgaatatcgcgccgacgctggaaatccgtccgggctatcgcttcaacgtcattgtcacgaaagacatgacgttttctaagccctaccaggcgtttgactattaactccaaggagtaacttatgaagaagctcgctaagaatgttttagccgctaaagtagctctggtgctggccctctcggtcggcaccttggcggtcacgcctgcgcaagcgggcattccggtcatcgacggcaccaacctgtcacaaaccactgtcaccgcgattcagcaggttgcgcaggtccagaagcaaatcgaggaataccggacgcagttgcagcagtacgaaaacatgctgcaaaacacggtggccccggccgcctacgtgtgggaccaggcgcagtccaccatcaacggcctgatgagcgccgttgataccctgaactactacaagaaccaggcgggcagcatcgacgcttacctgggcaagttcaaggacgtgtcctactacaaggggtcgccgtgcttctccctgtcgggctgctcggaaagcgagcgcaaggcgatggaagagaaccgccgcctggcgtccgaatcgcagaaaaaggccaacgatgcgctgttccgtggcctcgatcagcagcagagcaacctcaagtccgacgccgccacgctggagcaattgaagggcaaggcgacgacggcgcagggccagttggaagccctcggctacgccaaccagttcgccagccagcaggccaaccagctcatgcaaatccgtggccttctgcttgcgcagcagaacgccatcgccacgcagatgcaggcccagcaggaccggcaggcccagcaggacgctgcgggcgcgaagctgcgcgagggttcgtaccgcgcaagcccgtctaagacctggtgaggggaggcgcgatgaagaaatccaacttcatcgcagttgccgcgctggccgccgtcatggcggccagcctggcaggctgcgacaacaagcccgacaccgacaagctgacctgcgccgatctgccgaaggtcacggatgccgctcaacgcgcggagctgttgaagaagtgcccgcgcggagaaccgggaggcttcaagcccagcgaaaagaaagagtggtgatgacgtatgaaaatccagactagagctgccgcgctcgcggtcctgatgctggccttgatgccggtagcggcatacgcccaaatcgacaattcgggcatcctcgacaacgtattgcagcgctaccagaacgccgcgagcggctgggccactgtcgtccagaacgccgcaacctggctgttctggaccttgaccgtgattagcatggtctggaccttcggcatgatggcactgcgcaaggccgacattggcgagttcttcgccgagttcgtgcggttcaccatcttcaccggcttcttctggtggctgctgaccaacggcccgaatttcgcgtcgtccatctatgcgtccctgcggcagattgcaggccaggcaacggggttggggcaggggctttcgccgtccggcatcgtcgatgttggcttcgagattttcttcaaggtgatggacgaaacctcgtactggtcgccggtcgatagcttcgtcggtgcctcgttggcggccgccatcctctgcatcctggccctggtcggcgtgaatatgcttctgctcctggcgtccggatggattcttgcctacggcggtgtgttcttcctgggcttcggcggctcgcgctggacctcggacatggcgatcaactactacaagaccgtcctcggggtcgccgcgcagctcttcgcaatggtgctgctcgtaggcatcggcaagaccttcctcgatgactactacagccgcatgagcgaaggcatcaacttcaaggaacttggagtgatgctgatcgtcggcctgatcctgctcgttctggtcaacaaggtgccgcagctcatcgccggcatcatcaccggcgcgagcgtcggcggtgctggtatcggccagttcggcgctggcacgctcgtcggtgcggccgcgacggccggcgcggcaatcgcaactggcggcgcatctatcgcggccggcgctgcggcggcggccggtggcgcgcaggccatcatggcggccgcgtcgaaggccagcgataacgtctctgccggcactgacattctgtcgagcatgatgggcggcggcggtggcggcggcggtggtagcgccggcaccagcggcggcgacggcggcggctcgggtggcggcggtggctcgggcggcggtgaaaccccgatggcctcggccgccggcgacaacagcagcggcgcacgcggcggcagttcgggcggcggctcgggtggtggccgttcgtctggcggtatcggtgccacggcggccaagggcggccggatcgcggccgataccgtcgccaacctggcgaaaggtgccggctcgattgccaaggccaaggccggcgaaatgcgcgcatcggcccaggaacgcatcggcgataccgtaggcggcaagatcgcgcaggcaattcgcggcgcgggtgcggcggcgcagaccgctgcaaccgtcgccgatagcaacagccaggcgcaggaacaacctgcaccggcacccgcaccgtcgttcgacgacaacagcctttccgcaagcaacaacagggaagcggccgccgacgcggattccgaagtggcgagcttcgtcaacaagcccgcccaatcctgaaacgactcttaggagctacgaccatgcaactgaaaaaagcgttctcgtcggccgccctggtggtggccttgggcctcggcgcaactggctcggccagcgcgcaagacgtgctgacgggcgatacccgcctggcctgcgaggccattctgtgcctgtccacgggcagccggcccagcgagtgcagcccgtcgctctcgcggtacttcggcatccacaagcgcaagctgtcggacacgctcaaggcgcggctgaacttcctcaacctctgcccggtatcgaaccagacgccggaaatgcagacgctcgtttcctcgatttcgcgcggggccgggcgctgcgatgcgtcctcgctgaactccgtgctgcgtgagtggcggagctgggacgaccagttctacatcggcaaccgcctgccggactactgcgcggcctacaccggccatgcctataccgacttcaacacgaccgcgccgcgctacgtcggcacgccggaagagggcggctattggatcgaggcggccgactacgaccgcgcgctcaaggagtacgaggcgaagctgaaagagcggcagcagcagtacggtcgctatggcagcgacgcctaccgtcggttcgagcggtaaggggaggggatagcgatgccgtttgccaagctgctggcacggaacgctctgccggtggtcgccctggtggcggccactggcttcggtgcggcggatgcgaccgccgcacggctcttccccgatctgtcggaacagatggaagagcgcgttgtgtgctcggtgtctgcggccgcgaagtacgagattccggccaacattcttctcgccattcgggaaaaggagggcggcaagccgggccagtgggtcaagaacaccaatggcacctatgacgtgggcgagctgcaattcaacaccgcctacctgggcgacctggcgaagtatgggatcacggcccaggacgttgctgcggcaggctgctatccctatgacctggcggcctggcggttgcgcgggcacattcgcaacgacaggggcgatctgtggacacgcgccgctaactatcactcgcgcacgccgtcgaagaacgcgatctatcgcgccgatctgatggtgaaggccgacaagtgggcgaagtggctggatgcgcgtttcgtcaccgtcaactatggccccagctcgccggcgcagccggcagggaaggggaccacacttgcggccgctgatacgtcggcagcagcgccggccgaagcgcagccgatgaagcaaggccggatcacccgcaccagcctccgcagctcgggttacgtaccccggcagctcatcatcaacaacacgccataaggaggaacggccgtttagcggctaaagcctatgggcattcgcaacctgacgcagcgatacatgaacggggccagggcctacgcggcctgggcggcatcgcaggcgaaagcgccgtttgatcttctggtactgggcatcgggcctgtcatcgtctttggcctggtcgcgcatacgctgctcgcgttcctgcccacatgggccatgtacgccgccggcgctctgctggtcctcgcggccctgcctttggcgctgcacgtcctccgggaatacgcgctgcgctatgggcgcaaatagcgccctgcagggcgttcttactccaagggggagggcatgaatacacgcgccatgaacgacgccagcggccgggcctcgctgcctgccatggtgatcgccgacggcaccattgaagccttgaagtggctcgccttgcttcccatgaccggggatcacgtcaacaagtacctgttcaacggtacgctgccatatctgttcgaggcggggcgcttggccctgcctcttttcgttttcgtcctggcgtacaacctcgcccgcccgggcgcgctcgagcgcggtttgtacgggcgagcgatgaaacgcctgttggccttcggcctggtcgcctcggtcccgttcattgcgttgggtggagtggtgggcggatggtggccgctgaacgtcatgttcacgctgttggccgcaaccgcgatgctctacctggtcgagcgcggccgctcggtcgctcctatagcgctgttcgtcgtggccggcggcctggtcgagttctgttggccggcgctgctgctggccgcgtctgtctggttgtacctcaagcgcccgacgtgggcggccgcgttgatggcgctgctgtcttgcgcgtccctgtggtacatcaatggcaacctttgggcgcttgctgttgtgcccctggtgatcgtcgccgccggcgtcgatcttcgtgtcccgcgcctgcgctgggccttttacacgtactacccgctgcatcttgccgctctttggctgatccgcattccgatgcgcgaggcgggctacttgtttttcacctgacctttgagattccaatatgcaattgctcaagaaatgcaccatcgcggccctgccgctgctcgccctgtccggctgcgcactgctgaacatccccatgccgacgccgcccggttcgaccccgccggaaatgctgaccgtgccagtggcgcaaatctgccgcgacgctgacaagaaccctgttcgggcaacggagctgtacggcaagaaagggttgtcggccaccggcaaggtgcaggtgatttccgaaggcttcaagcctcgctatcgggtgctgctgccgcctggcagcgcctcggtccatgctgggaccgataaccagctcgccatcaagtcggtttccaccggccagaccacgcgcgtcactggcaccgtgaaggacgtgtcctacgaccataacggctgctcgatctcgcttgacgatgcgaagttctactgaggggagggcggcggatgctgacacggttgaagggcttccttgctcgtcgccgcgagttgaaggaactggatgtgtccgtggtgagccggccccggccggctccggcggaattggtccaggttgatgcacgcgaggccgtttggcgcgtgccggtgcccggccaggccgaccgcttcatgtcggccaagcctggcgcgatcaacgatgaaatgttcgtggttcgggtggacaccgaagcgttctatcgggcttggctgcgcagcagctcgacgggccgcgaaacgcggtcggacaactgcccgctgcgctcggaaatgccgcaggactacaagttcaagcacgccgtccagggcttcgcgcacggcagggaaaatcctgtgccgctggccttcgccggcgcgcaccaggagcgccaccgggtggacattggtttcagcaacggggtcacgcgctcgttctggctgattgccaacaaggctccgtcgttcccgatccaggtccacggccgggagtcggccgagctgctgaacaaggtttgcggcctcgatcctgcgccgctgtcgttcacggaactgttcgcgcaggcccaacgccaggctccgcaggtcgccacaccggcccggcctgcgccggcagcggccacccggccagctcccaaggtgcagccacgccccggccgaagcggcccgcgcaaaggccgcggactctgactacaaccgtgcgcaaggcgcattagggaggatgtatgtatgtaatcgcctgcggcatcgttgccggcttggcggctgcggtggccctgttgggcttcacgccgatgatggaggcgcttgccgccggcgaacgccgcaaggcactcgcgcaatggacgcggacgatgttcctggtgctgctgcctgtcgtgctgatgtgcgcgcccatcgggtccagcatttacgacgccgtgcaagcggacgctggcaagcccatcgctttccacaacggccggatcacggtcgtcatggccctggtcggcagcttggccgttgtcctggtcgcggctgcgcgtgcggtggtcaaccgcaagcatgccagcttctggttcgtcggctgggtgatggcgtcggttttggccggcggcgtcggcgcgatcgccagcgcgaagcaactggcgttcctcggcgaacatagcggcatggtggccttcggcttcttccgcgaccaggtgaaggacatgcactgcgatgcggacgtgatcctggcccggtgggatgaaaaggcgaactcgccggtggtctaccgctgcccgaaggcgtacctgctcaacaggttcgcatccgcgcccttcgtgccctggccggactacaccgagggggaaagcgaggatctaggtagggcgctcgcagcggccctgcgggacgcgaaaaggtgagaaaagccgggcactgcccggctttatttttgctgctgcgcgttccaggccgcccacactcgtttgacctggctcgggctgcatccgaccagcttggccgtcttggcaatgctcgatccgccggagcgaagcgtgatgatgcggtcgtgcatgccggcgtcacgtttgcggccggtgtagcggccggcggccttcgccaactggacaccctgacgttgacgctcgcgccgatcctcgtagtcgtcgcgggccatctgcaaggcgagcttcaaaagcatgtcctggacggattccagaacgattttcgccactccgttcgcctcggcggccagctccgacaggtccaccacgccaggcacggccagcttggcccctttggcccggatcgacgcaaccaggcgctcggcctcggccaacggcaagcggctgatgcggtcgatcttctccgcaacgacgacttcaccaggttgcaggtccgcgatcatgcgcagcagctcgggccggtcggcgcgtgcgccggacgccttctcgcggtagatgccggcgacgtagtacccggcggcccgcgtggccgctacaaggctctcctggcgttcaagattctgctcgtccgtactggcgcgcaggtagatgcgggcgaccttcaaccttcgtccctccggttgttgctctcgcgtcgccatttccacggctcgacggcgtgcggatcggaccagaggccgacgcgcttgcctcgcgcctcctgttcgagccgcagcatttcagggtcggccgcgcggccgtggaagcgataggcccacgccatgccctggtgaaccatcgcggcgttgacgttgcgcggctgcggcggccggctggccagctccatgttgacccacacggtgcccagcgtgcggccgtaacggtcggtgtccttctcgtcgaccaggacgtgccggcggaacaccatgccggccagcgcctggcgcgcacgttcgccgaaggcttgccgcttttccggcgcgtcaatgtccaccaggcgcacgcgcaccggctgcttgtctaccagcacgtcgatggtgtcgccgtcgatgatgcgcacgacctcgccgcgcagctcggcccatgccggcgaggcaacgaccaggacggccagcgcggcagcggcgcgcagcatggcgtagcttcggcgcttcatgcgtggccccattgctgatgatcggggtacgccaggtgcagcactgcatcgaaattggccttgcagtagccgtccagcgccacccgcgagccgaacgccggcgaaaggtactcgaccaggccgggccggtcgcggacctcgcgccccaggacgtggatgcgccggccgcgtgtgccgtcgggtccaggcacgaaggccagcgcctcgatgttgaagtcgatggatagaagttgtcggtagtgcttggccgccctcatcgcgtcccccttggtcaaattgggtatacccatttgggcctagtctagccggcatggcgcattacagcaatacgcaatttaaatgcgcctagcgcattttcccgaccttaatgcgcctcgcgctgtagcctcacgcccacatatgtgctaatgtggttacgtgtattttatggaggttatccaatgagccgcctgacaatcgacatgacggaccagcagcaccagagcctgaaagccctggccgccttgcagggcaagaccattaagcaatacgccctcgaacgtctgttccccggtgacgctgatgccgatcaggcatggcaggaactgaaaaccatgctggggaaccgcatcaacgatgggcttgccggcaaggtgtccaccaagagcgtcggcgaaattcttgatgaagaactcagcggggatcgcgcttgacggcctacatcctcacggctgaggccgaagccgatctacgcggcatcatccgctacacgcgccgggagtggggcgcggcgcaggtgcgccgctatatcgctaagctggaacagggcatagccaggcttgccgccggcgaaggcccgtttaaggacatgagcgaactctttcccgcgctgcggatggcccgctgcgaacaccactacgttttttgcctgccgcgtgcgggcgaacccgcgttggtcgtggcgatcctgcatgagcgcatggacctcatgacgcgacttgccgacaggctcaagggctgatttcagccgctaaaaatcgcgccactcacaacgtcctgatggcgtacttacccaaagaacagctaggagaatcatttatgctcagcacacttccacaagctcatgcaactttcttgaaccgcatccgcgatgcggtcgcttccgatgttcgcttccgcgctcttctgatcggcggctcttacgttcacggaggactcgatgagcactccgatttggatttcgacatcgttgttgaggacaactgctacgcagatgtcttgtctacacgcaaggattttgccgaggcactgcccggcttcctcaacgcgttcaccggcgaacatgtaggagaaccgcgccttctgatctgcctatatggtccgccactgctacacatcgatttgaagttttctcttgcttccgatctcgaccagcaaatcgagcggcgggcggttctgtttgctcgtgatccggcagagatcgagaagcgcattgaggcggcagcggtggcatggccaaaccgtccctccgagtggttcgaagcacgttgtcagcgccagtgatataagacggtaattcaccatttggattgtccgctccacccaacatgttgtttccttaaggttctcacaccagaaaggacatcaacatgctgagcagagaggacttttacatgataaagcaaatgcgccagcagggcgcgtacattgtcgatattgcgactcagattggttgctctgaacggacggtcagacgctacctcaaataccctgaaccgccagccagaaagacccgccacaaaatggttaagctgaaaccgtttatggattacatcgacatgcgcctggcagagaatgtctggaatagtgaggttatctttgcggagattaaggcaatgggttatacgggcggacgttccatgctgcgttactacatccagcccaaacgtaaaatgcgtccgtcaaaaagaacagttcgcttcgaaactcagcctagataccagctccagcacgactggggcgaagttgaggtggaggttgccgggcaacggtgcaaagttaactttgcggttaatacgctggggttctcccgccgcttccatgtcttcgccgcaccaaaacaggatgctgagcatacctacgaatcactggttcgcgccttccgctacttcggtggttgtgtgaaaacggtgctggttgataaccagaaggctgcggtgctgaagaataacaacgggaaagtcgtgttcaactccggattcctgttgctggccgaccactataacttcctgccacgggcatgccgtccacgcagggccagaacaaaaggtaaggttgagcggatggtgaaatacctcaaggagaacttcttcgttcggtaccgcaggttcgacagcttcactcatgttaatcaacaactggagcaatggatagccgatgtggctgacaaacgggaacttcgccagttcaaagaaacgccggaacagcgcttcgcgctggagcaggaacatctgcagccgttaccggatacggacttcgataccagttacttcgacatccgccatgtgtcctgggacagctatatcgaggttggtggtaatcgttacagcgttcccgaagcgctgtgtggtcagccggtatcgatacgaatatcgctggatgacgagttgcggatctacagtaatgagaaactggtggcctcacatcgcctctgttcggcatcgtctggctggcagacagtgccggagcatcacgccccgctctggcagcaggtcagtcaggtggaacatcgaccactgagtgcctatgaggagctgttgtgatgcatgagctggaagtcctgctgagtcgcctgaaaatggagcatctgagttatcacgttgaaagcctgctggaacaggcagctaaaaaagagctgaactaccgggagttcctgtgcatggcgctacagcaggaatggaacggcaggcatcagcgcggtatggagtccaggctgaagcaggctcgtctgccgtgggtcaaaacgctggagcagttcgactttaccttccagccgggcatcgaccgtaaggttgtccgggaactggctggtctggcgttcgtggagcgcagcgaaaacgtgatcctgctgggacctcctggtgtcggaaaaactcatctggccatagctcttggcgtgaaagcggtggatgcgggacatcgggtactgtttatgccactggacagactgatcgcgacactgatgaaagcgaaacaggaaaaccggctggagcgtcagctgcagcaactgagttatgcccgggtgttgatcctggatgaaataggctatctgccgatgaacagagaggaagccagcctgttcttccggctactgaaccgtcgatatgaaaaagcgagcatcatactgacgtcaaacaaagggttcgcagactggggagaaatgttcggagatcacgtgctggcaacagcgatactggatcggttgctacatcactcaaccacgctgaatatcaaaggagagagttaccggttaaaagagaaacgtaaagctggagtgctgaccaaaaacacaacgccaatcagtgatgatgaaatggtgaaaagcggacagcatcagtaacgaaagtatcttagcgggcatgaaaatggcaaataacggtcaaacatcgtggcgttgacaacgtgcctggatctggctacactatgcggccaccaagctcgcccgtggagtctctacgaagcgatcggcatgctcggtttccttccgtgagcaagtgttaggacctttgctctaccgtcgcgctggaaaggaccagcgcggagtgaggcgattggaaacccttcgactgggatgaagagcgcagacctagccaccaccattgcgctgcacgatgcgttgtctgtcagggatgcttaattaaggaagatctagggtaatcccggatcttcgtcacaattctcaagtcgctgatttcaaaaaactgtagtatcctctgcgaaacgatccctgtttaagtattgaggaggcgagatgtcgcagacagaaaatgcagtgacttcctcatctggcgcaaaacgagcatacagaaaggggaatccgctttctgatgcagagaaacaaagattatcagtggcccgtaaaagagcttcgttcaaggaagtaaaagtatttcttgaaccaaagtataaggccatgctcatgcaaatgtgtcatgaagatggcctgactcaggctgaagttctgaccgcactgataaaaagtgaagcgcaaaaacgatgtgtgtgatgatgggcttacattcttgagtgttcagaagattagtgctagattactgatcgtttaaggaattttgtggctggccacgccgtaaggtggcagggaactggttctgatgtggatttacaggagccagaaaagcaaaaaccccgataatcttcagaaagtttggcgactgagaagattaccggggcccacttaaaccgtatagccaacaattcagctatgcggggagtatagttatatgctcggaaaagttcaagacttctttctgtgctcgctccttctgcgcattgtaagtgcaggatggtgtgactgatcttcagtcgagggaacggactggaaacagacgtactgacatcccaggaaacgatcttgaaacgtaaaccgtgcgccaacacaggttacgttcataaagtaagtcgctgattttagaaatctgtagtattctctgcaaacgatctaggtttgatccttgaggagacagagatgtcgcagattgaaaatgcagtaacttcctcatcgaaacgcatttacagaaagggtaatcccttatcttccgctgagaagaagagattatctatttcacgaaaaaagacgacgcataaagagctcaatgttttcatacaaaacatacataaagaaagcttgcagcagctttgtgaagagactggaactactcaggctcaaatgattgagctactaattgaacgggaaatggctaaaagagcctgagataagaaggtgaatgagtaactttcttgatcgtctcgtcagtgagtgttagattgctgatcgtctaaagaattttgatggctggccacgccgtaaggtggcagggaactggttctgatgaggtgcctacccgggaccagaaaagcaaaaaccccgataatcttctcatttcttggcgggaacgaaagattaacggggcctacttaaactgtatagccaccaatcaggctatgcagggagtatagttttatgctcagaaaatttcaatacttgtttctgtggcatttactccttccgtgcattgtaagtgcaggcagaagtgactgacacccgaacactgttcactcattaccgacaggggatccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgctcgacaagctgtggtcagcagaacagacataacataagctggagcatgtatataagctgtagtgagtaagtcatgttttatgaagggagcaatgcctcagcatcaggttacggggtgactcacgtaagggaccggcagatgacagctcagctacaggcagcactgcaggaaactgaatataaactgcagtgagcagaccactcactgcacctgataatataagctgtagtcagtaaaggaacactcttcactgactacagtttatgttcagcgggatttgaagagtttttccaggtcatccagcgtgatgcccagtttctcaagcagggcaagcttctccgccatctccggactgactttttccgcaggtgaaggtggcaacggattttccttactctcatcattcggtgcttttaaccggggacgccggtagtgaatgcagaagagttttgtccgcccccgctggatctccgtgtaatcaagatatccaatctcgcgcagctgctccattgcccgtctgaccgtctggttctgggaaaatacaggagacttcagattgaggcgtgcacgcagccgcgccagcgataccggtgccggatcccggggcaggctctctataaaggtgtagagtgcctgggcggactcccgtcgcttcagggcattaatcgccttaagctggagaaggacctttctgtcaaactggtacagttcaaaaaggcggggatcagcctgtaactgaacaatatcccgttcagtatcgtagtaggctgactgtaccagatgggtgatgtattcccgggtgtgcttctcatcggtgcgggaaaaggagatcacggtaccggcaatgcgtttcagggaagggctgatgcgctcacgcagcctgcgtgatgactggcttgaaggtataccacacagttttgcaaactcgacaaaaggcagttcaactttgtcaccaattacgttatggcgggcaaaggaatgaatgatccccacccaggtcttgaaatcgttatccatatccaggcgggggccggtgatctcaaccttatcgaatccctcagcacgggccagggaaagacgtgtcagctcttccgtggcatcagtacgtgacagtgtattttttttactgttcttcagtgatttaagggtcggtacaaaaacgcccagacgcatcagcgccacaggttgtacggtgttgttattgtttggtgtcagtgtcaccagctcaccggacgacttatccacctcgccgaacagttttttgatgtctgaattttcgttttccagaaagcctcctgcctgtggacagtaaaagaataaacagcaaatcgtgtgtcagagccccgctaactgactgaagaaaaacctgcagcctgtatttaacagctgagtctggcgtactgaccatagtttatgtcattaacttacttcagtatatggattactgaatacagcttatcattagcatgttacagtttatcagtaacatgccactgcttatgtgatcgtggtcccggcccatgagtgacaaggtttgcagcggtatggggatctttaatggatcctttgttgatcattcatggatctgtttactggatcgagcctcctctagccgcttgaggctttctgcctcatgacgtgaaggtggtttgttgccgtgttgtgtggcagaaagaagatagccccgtagtaagttaattttcattaaccaccacgaggcatccctatgtctagtccacatcaggatagcctcttaccgcgctttgcgcaaggagaagaaggccatgaaactaccacgaagttcccttgtctggtgtgtgttgatcgtgtgtctcacactgttgatattcacttatctgacacgaaaatcgctgtgcgagaggtacccgatccagcgagacaagccagatttcccctctttccatgtcagatttcctcctgaccagtcgccggtgcatccagccattctcgttcttcagctgatatttcagcattcggatcacactgtgccagtagctcagccagtgaatattgcgggcgtctgtacggctcaacaatcagccggccattatcaatgaccatgccaacttcattatctgtgcccagagacagcgcattcagcagtgccggtgggacggtcagcataactgagccgccaaccctcttcagtcgggtggtatgcattcttcacctccataaaagttatatttaaatataacatccactaaaaaaacacaccaggctttaacgcacaatgtttaataaaaatataactttcaaccaaacagtaaacccagcgtggtgctagaccatacgaaacgggaatgcggtaattacgctttgtttttataaggcagattttaatttttattggttaacataacgaaaggtaaaatacataaggcttactaaaagccagataatagtatgcgtatttgcgcgctgatttttgcggcataagaatatatactgatatgtatacccgaagtatgtcaaaaagaggtgtgctatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtaagcttaggaaaagtgaatgtggtcagcgtgctggtatatgggctatgatgtgcccggcgcttgaggctttctgcctcatgacgtgaaggtggtttgttaccgtgttgtgtggcagaaggacaaaagccccgtagttaatttttcattaacccacgaggcccctgcatgcttagacaacatcaggatagcctcttaccgcgctttgcgcaaggagaagaaggccatgaaactaccacgcagctctcttgtctggtgtgtgttggaattcaccgggtagggataacaggggacgtccagggaggcgttcaggacgactcacaaagaaagccgggcaatgcccggctttttctgctgctacctccgtagtcgtaaggtcgttgcaggtgctcgggtgcggtacaactcgccggtcgccagctcaagcgcgatcacgtcgttgccgtcgtagttgacgatgatgctgttgggccgactgtcctcacgcttcgcagggagaggccagccttcaatcgaagccggcgcaagctcgtagtgcttcccggtttcgacgctgcgcagcgtccaggtcctgcaaccggccacgccggtcgcagaaaccacggcgagcgagccgcgaaaatcgtgcgggtacgcctcgatgttcatacgcctcctagatcgagcgcgagcgtttctgctcggccttggccgcctgttcctgggacacctcgccgatgaccttgccctggccccggctgtaggcgatttcgtagttcttgccgacaaccggcggcttctcaaagatgccccggctgtgtttcacgatcccgccttcgctgaactggtagacgttgcgcccatcgtcgtgcagcacctggccgacgtgcttgtgcgggtggacgtttttgcttgcgtccttcgcatcgctcaactggtgaatgcctttcggtagccccgcctcgggcaacaccttcatggtcagccattcgccgttcactacctggtccacctggcggctgccgttcatgacggcgatcttgacgctgccctcgggtttcatgatgacgccagggcttgccgatgtgcgtggtgccccgatctgtactttgttcatacgctctagttctccttagtaggttctcgcgcggcgttgccgctgttcttgctgctcgatgtcttgctgcttgagctgctgcaccttctgccgctggccctcgtcgagaagcaccttgccgacagcacttctcacctggcgttcaaccccgtccttgcccaggctgctgcgctcggacaggtcgttgaaatcggtgtgcttcttcatgttcgacagggcggcgagctggccatcgttcaacagcgattccttgagcttggccgtgtcggcctcgctcaactggaccttgccggccgccgcgtccgcgaggcgcttttcagcgtgcaaatggttgcggtagttctccggggtgatcggcggcagctccttcgggtaggcgttctcgcccggcgcgaagattgggaagatggccttgccgccgaccgccttggcggcctcctgtgccttcgtcctgccgggattcacgccctgggtgatctgcacctggcggtcgtcgtcgccggcgatcacaacgggcttgtccgggaatttcgcgtgcagggcctcggcaacagcctgtaggttgccggaatcgaacgcggcgacagtcgcgtgccccagcgcttcggccactgtggcggcggtggcatagccttcgccgatcaccagcgccggcgcggccgcgagcgcatccatgccaccgacgacatggaagcatccttccttgcggctgtccttggcgaagcgcttggtgccgtcctcctggatgtactgcatggtccattgcttgccgtcggcgtcgtaggccgggatgtaggttttctggccctcctggtcggtaaggacgccggcgtgcacctgtagacccttgtcgcgcaggtacggcgtcggttccgtgatgggaaccaggctttgcgcctggcggccgatgcgctgcgccgtggcttcgtgctggcgttcttgttcctcggcacgcgcggccagcttggccgccgcctcggcctgcatcttggccttctcggcggggtccagggcgtagcccttggccttccacttcatttcgacgccggtgcggttgtttttgatgtaaccggccgggtggccgtcgaggtggccgacgtagaagcccgacttctcgcccttcttgtcgccctcggtctcgatgcggtgcttcttgccgtccatgatggggtgctcgccgcctggggtgacgacgcagcccatgcttttcagggcctccgcgaactcatcttcgggggtgacggccggggattgctgggtgggcacgttgtccggcagccagcgttgcagcttgcccatgtcggcgttcggtccggcgtaccaggacttggccaccttgtcccactgcgcgccggccgccttggcaacctggcgctcgccgtagggcacggccaggtagacgcgctcctgggccgcgttggggcgctcggccgtgggttgggtaggctgggcctcggcgcgggcctctacggccgctgtagcgccctcgcgcgcccatttggcgaacggggcagggtcaacccctgccggaacgtaccaggcgcgttcctggcggtcccagcgcgctccaagggccttcacctcgtctttctccttgaacggcacgttcaagtaggcgcgctcgggcttggcgggggcttgagcggccgccggctgctcggcggcgttcatggcctgggccatttcctgctgctcgcgctcgtagtcggcgatccggcgctgtaggtcctcgtcgtgcagcatggccgtgccctcggcggccttgcgcgcctccttggcggcaacgcggtcctcgtcggtgctgttgggatcgcggcgaactcgctcttcatggatgcgggcgaacttcgcggcctgctcgtactcgttggccgtcgcgtaagcgtcgatcacggccaggcggtcggcgagcgcttcggcattggtctgcgcgtcggggccggcgaagtcggcaagccattggtggccgccccacgcatggttcgcatagacgccccaaaactccggctctcggtcgccggccggcaccacggaccgttcgccgtcgtgctcgacctcgacgttggcctggacctggacgcggccggtccaatcggcaggcagctcaaagcccagcgtggtttcggtcagcgcggccagcgattggttgccttccgccggctccgcgccggcgcggtacatgcgcagggtctgcgcgatcagctcgtcggccggcgcaatggccggtcgtgccacctggtcttgctgttgctccatagttgccccctgcgcaggctcgatggcctgctgggtcgtttgttcttgaatttgcttctgctcgaacgccaggacgaaatcctggatcttctccgcgtcggcggccgcgcggaaaatctctagcgggtcctcttgtagcgccttgatccacgatccgacataggccgcgtgctggccggggtcgtggccgatgcccagctcgtcgcccaggatcatgctggcaatctcggcccgcagctcttccttggcgtacccctcgctcccgaagggatgcgccaggtcgcggtccagccgcgacgggtggccggtccagtgccccagctcatggagcgcggttgcgtagtagttgtcggcgctcgggaactggcctttgtcgggcagatggatgctgtccgtggacggccgataaaacgcgcggtcgtgctcgccgtggcggatggtggcacctgacgccgcaaggatgtgctcggcccgctcgacggcgctccaagtctgttccttgcgttccaacggcggcaggccgtcgatctgctccgcattgaacacggtggcgaagaacacgcgcgggcgttcgagctgcaccgtcaccttgaccggatcgccgttggcatcgaggaccggcttgccggtctgctcgtcggtcttggtctgctcttcgctgaacttccaatactggatcggcgtgcctttctcgccgcgacgcacctgtgcgccggcggcagcggcctgcttgtaggtcatccagcgcgggtccgcatggccctgggccatgagctgaatcgcgttgatgcccttgtaacgcttcccggtagtcgggttgagcgggatgaaggagccgggcatgcccggttcccacggtttttgccacggcgcagtgccggctttcagttgctcaatgaggcgttcggcaacctgctcgtggaacggctttttgacctctgccatagccaattacctcccgtcattggcggccgcggtcgtcgtgtcctcgggcacggtcgcgtccaccaggtcaatgtcgctctcggcggcgtcctgctcgctcaaggcgtcctcggggaaggccccggccttctccgcttcttccgggtcgaactcgacctggaagccgggcgtcatcgcatcggcgagcttttcacgcagggcggcggcggcgttcgcggtatcgtcgaacggcgcaaagtcgtcctgctgcacggtcttgggatcattcatcgctttcactcctggttggtgccgttacggcctttgctgtagtccggcctgcctttcaggtcggggtatgtctgcttgcacgtcgggaagttgctgcacccccaccagaacatgccgcgcttcttgccaggccgacgggaaaggccgtggccgcaggccatgcacttgtgcagctcggagactttcggggcctcgcgcgggacgggcttgccgcccttgtcgtcgcacgcgaacttgcagccgtcggcaaagccggtgcagccccaaaagtattcgttcttgtccttcttcttgaggcgtcgcagcggcttgccgcaggacgggcaagggtgcgtgtcgatcttcatgttgaggccgttgtctttgatgttggcgacctcggcgccgatgtattccatcagctcgttgacgaacgacagcgtgtcgcgctcgccggcctggatggccttctgctgctcatgccagagcgcggtcatgtcggggaatctggccgtgtcgggcagtgcgtcgtacagctcttcgccggtcggcgtggacacgatgtgcttgcccttctccaccaggtagccgcgctcgaaaagcgtggcgatgatggagtctcgcgttgccggcgtgccgatcccgccgtgctcgccttgcttgcccttgtccttttcgatcaagattttccgcaggcggtcatcgcggatgtatttcgcaacgcgggtaaggtccgacagcagagattccatcgtgtacagcggctgcggtttcgtctcctgctgctcggccttcgcatcggtgcaggtgccggcctggccgtcacgcagcttgcgcaggtcctgttcaatgtcgtcggcattgccttccaggtcctcgttgccggcgtcgttcttgtagagaatcttccagcccggcgacgtggtgacgttcgagcgcacgccgaaacgatgatcgccgacctgggcaagcacgtcggtctggtcatacagatgcttcggccagaactgcgcgacgtaggcgcgcgcgatcagcaggtaaatcttctgctcggcatcggtgagcttcgacaggtcggccgtgctttcggtcgggatgatcgcgtggtgcgcggaaaccttggacgagttgaaggcgcggctcttgatcgtcggattggcgcgctgcgcagcagcggccagcatgggggccgtctgtgcgatggccgccagcacgcccggcgcatcgccgtgctgttcctcgctcaagtattcgcagtcggaacggttgtaggtgatgagcttgtgcttctcgcgcagggcctgcgtaatgtccttcacctggtccggcttgaagccgaacttgcgcgaggcgtccatttgcagtttcagcaggttgtagggcagcggcgcagccgcttccttcgccttggtggtcacggacacgatgcgggcgggttggccgctcacggcggccgcgatgccctcggcgtgctccttgttgctgaggcggcctttctcgtccaccggatcgccgtcggcgacctggtaacgggccgggaactgaatgccctcgacctcgaactggccgttcaccaggtagtagtaggttttctggtgggccgcgttctcgcggcaacggcgcacgacaaggcccaggatcggagtctgcacgcgccccacgctcaacagcccctgatagcccttcgcgcgtgccgcaagcgtgtacaggcgcgtgatgttgaagccgtatagctggtcgccgacgctgcgggcctcggccgcagcggacaggccggcgaactcgcggttgtcgcgcatcgcggcgagctgccggcgcacgatcttcacgttgttgtcgttgataagcagccgctgcaccggcagacggcagttggcgtattccaggatttcatcgaccagaagctggccttcgtcgtccgggtcgccggcgtgaaccacgcttttcgcctgcttcaacaggctgaggatggtcttgaactgagctttcgcaccggcatcgccggacggtttcttgcgccagggaatatggacgatgggcaggtcggccatgttccagttggcgtagcgctcgtcgtagtcctccgggtctagcaaggccagcatgtgaccgtagcaccaggtcacgcggtcggagccgcattcgtaatagccgtccttgcggctgccgccgcccaggccctcgacgatggcttttgccagctccggtttttcagcgattacaaggcgttcaaattgcatatatccccctaccctcaccaggtcagaaccggcctgatgacggtgatgatttgcgaacgattgacaggcccgaagtagcggccgtcgaaagacgtgtcgcttacgtcggacataagcagaacctcggcggtccccagggtgtagctgtcggactgataacgaggcagcggccgtcctgatggatcggccttgatgagcgcgctgtgaggcagcagcccgccattcacgcgcacgccggcgtcggtgatggcaacctcgtcgcctttagcggctaaaactcgcttcatcatgtagccgtagtcgccggggcagaaaccgccggcgatgtagccccgctccttggcgtccgaaaacacgccgacttgcggcgggcagaacatgacgtaagcccccttctccaccggcgcattcgatttccagtacaggccgaccggaatgcttttggtggtgttgaccttcgcgccggcgagataggccgcgccggcgagcaacaaggccgcgccgcctccgatggcgacgtacttggtgaggcgctggaagcggctcatatcgtgatcccctccccttcctcgacggtggccgtctggatcagcttgtcgctgaccttcggagccggtacggccgcgcgggcctggaatatcgggtctttgaagtagagcggctgcttgccgtagatcgcgggatagccggcgacgtacacaaccatgtcgcccgcctcttcaatgctgccgtcggcgctcttcttcggccccggcatgcgcaggcattcatcgggggtcagcaatggccgctgcacttcctggaaggtccgcgagacgttgcccaacagcgccgacgtgcggcggccgctcgtcgtgatctgctccttcacgatggtcgtggtgcctgtcagttttgacaggtgctcggccgtctccacgcggttcggcgggtaggcgttctgcacgtggcagttcgacgtgatgctttcgtcgtggccgtagccggtttcgcggctcttgagctggttaatgtcctggcagatgaggtagcacttgatgccgtagccggcgacgaaggcaagggactcttgcaggatttcgagcttgcccaggctggggaactcgtcgagcatcatcagcagacgatgcttgtagtgcgcgacaggacggccgttctcgaagtccatcttgtcggccagcagccggacgatcatgttgaccatgacgcgcaccagaggccgcagacgggccttgtcgttgggctgcgtcacgatgaacaggcttaccgggtcgtcgtggtgcatcagttgcttgatgcggaagtcggacttgctgacgttgcgggccacaaccgggtcgcggtacagggccaggtaggacttggcggtggacagcacggaaccggattcttcctccgggcggtccatcatgtcgcgggccgcagagccgaccgcagggtggttctgcccgtcaacgtggccgtaggtggtcatttccatccaaagctcgcccacgtcgcggttcgggtcggcaagcatgccgtccaccgacggcagggtggccggcgtaccctcgttcttagccttgtagagcgcgtgcaggatgacgccgacaagcagcgcctggctggttttctgccagtgcgattccaggcccttgccgtccggatcgacgatcagggtggcaaggttctgcacgtcgccaacctcgtactcggtccccaagcggatttcatcgagcgggttccagcacgcgctaccctgcgcggatgccggctcaaagcgcacgaccttgttgcgggcatgcttcttccgccagccggcggtcagcgcccacaactcgcctttcaggtcggtgatgacggcgctgtgcgcccaggaaagcagcgtcggaacgaccaggccgacgcccttgccggagcgcgtcggcgcgtaggtcaagacgtgctcggggccgttgtgccgcaggtagtggaacttgccgtccttgtcctgccagccgcccacatagacgccgctggaagtgggcgggtgtttgcctgacaccagctcgacgacggtgcgcggccggggcagcaggccggcggcctgtatgtccttcttgtcggcccagcgggccgaaccgtgcagatagtcgttcgccttgccggtgttcgccttgaccatctgcgtgacggccgtgcccagcaggcccacggtcgaaacgaccatacccatgctggccgcgcgcatgaaatcgtcgggatattggccgtaccacttgccggcccattgaaggatcgaccagggcgtgtagacgtggttgatattccagccaagtccggcctgatactggaaggaatgggcgaaatattgcgtcgcggtctgcaagcctgccccaagggacaggccggcgaggatgggaacggtcttgctggccttcggttttttcgcccgtatctgtggccccacggcgttgtttcggttcttcatctactcctacctcgggtagttttaagggagcctcgcggggtcacggtgacgggatcaccgatggcgaggcgcttcatgcgttgcaccgtggccttatcgacgggcagcaccagaatctcgtcgttttctttcctcaacagggccagcgcctggtcctcgacgttccgggtgcctgcataggacagcgcaccaacataatcagtatatcgtgcatgcttcggtatatcgaagccgtttagccgcttttgctcgcgctcggcaacatatttctcggccgccgcgatctgttcgggctttagccctcttcctggcccagaaactccccgtcgcagtgcgtgagctggttcggctccttgctgctccacgtgaccaggaacatcacgcggcaatagcatttcagctccgccggcgatgcgaaccacaccgagttgggacagcgctcgcaaacggttttggctttggggcggcggctttcgtccaatgcgtccaacgttgggcttgcggagtgcgacggttccgccggcgctgacggcgcgagcgtcccgtcggtcgccgtcgccgcctgtggcgttgagggtggttctggctgcggcaggtcgaatgcctccatcgccgccgcgatctcttcgtccgtcatttcgttcgggttgctcatgtgcttgctccttcgtcagtagttcttgacggcggcgctcaagggcggcgtcgtcaaaggtgattgccagacggccagcggcggccgcctgcgcgatccgctccttgaactctgctgtgccgttgacggtgatccggtcgccgaagcgctccattgccaggcgcagggcggcgtccaggccgtccgtggtggcctcgcgcgagacttgcaggcggtcgccgtcgtcgcggacggcgctgctgccgacgcgatagatgatggttcccttcttcgtgatgttgtccgtcacggccgcatggcccggcttggcctcgccgctgccctggatggtgttgcccttgaggtcgctgcggccctcgcgtgcgcgcagcgcggccagggccttgtcgtcgcccttcatcgcctcggccttgagccagtcggcccacgcgcggcgctgcgtgcgctcctggaccgcctgacggccctgccggtactcgcggttgatcttgtccaggtcggcgcgcagagccttgtgcgcctgcgcgtacatcagtcgctttgcaatgcgcccctcgcccagcagcttgatagcggcgcggcgcagccggttgctgcgcatcgcggcttcaatcaggcggtcacgacgccggcgcagcgtgtccagctcgcccttgcgcacggcccccatttcctggcgttcagactgataccgggcgtatagctcggtggtgtcgatgcgggtcttgagcggcttcgctcgatactcccgccgccggggggcttcgccgccctcggctggcgtgaatgccccgaatcgggcttcgagcttcggcttggacaggtcgcgcgaaacggtgctggccttgaccgtcgtgccgtcgccggcctcgaagatgaagccgtttccgcgctcgcgcagcttaagcccgttttcccgcaggacgcggtgcaggtcctcccaggattgcgccgcttgcagctccggcaggcattcgcgcttgatccagccgaccaggctttccacgcccgcgtgccgctccatgtcgttcgcgcggttctcggaaacgcgctgccgcgtttcgtgattgtcacgctcaagcccgtagtcccgttcgagcgtcgcgcagaggtcagcgagggcgcggtaggcccgatacggctcatggatggtgtttcgggtcgggtgaatcttgttgatggcgatatggatgtgcaggttgtcggtgtcgtgatgcacggcactgacgcgctgatgctcggcgaagccaagcccagcgcagatgcggtcctcaatcgcgcgcaacgtctccgcgtcgggcttctctcccgcgcggaagctaaccagcaggtgataggtcttgtcggcctcggaacgggtgttgccgtgctgggtcgccatcacctcggccatgacagcgggcagggtgtttgcctcgcagttcgtgacgcgcacgtgacccaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggcttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcattgggtgactcctgacggttgcctctggtgttcaaacgtgtcctggtcgcttgccggctaaaaaaaagccgacctcggcagttcgaggccggctttccctagagccgggcgcgtcaaggttgttccatctattttagtgaactgcgttcgatttatcagttactttcctcccgctttgtgtttcctcccactcgtttccgcgtctagccgacccctcaacatagcggcctcttcttgggctgcctttgcctcttgccgcgcttcgtcacgctcggcttgcaccgtcgtaaagcgctcggcctgcctggccgcctcttgcgccgccaacttcctttgctcctggtgggcctcggcgtcggcctgcgccttcgctttcaccgctgccaactccgtgcgcaaactctccgcttcgcgcctggtggcgtcgcgctcgccgcgaagcgcctgcatttcctggttggccgcgtccagggtcttgcggctctcttctttgaatgcgcgggcgtcctggtgagcgtagtccagctcggcgcgcagctcctgcgctcgacgctccacctcgtcggcccgctgcgtcgccagcgcggcccgctgctcggctcctgccagggcggtgcgtgcttcggccagggcttgccgctggcgtgcggccagctcggccgcctcggcggcctgctgctctagcaatgtaacgcgcgcctgggcttcttccagctcgcgggcctgcgcctcgaaggcgtcggccagctccccgcgcacggcttccaactcgttgcgctcacgatcccagccggcttgcgctgcctgcaacgattcattggcaagggcctgggcggcttgccagagggcggccacggcctggttgccggcctgctgcaccgcgtccggcacctggactgccagcggggcggcctgcgccgtgcgctggcgtcgccattcgcgcatgccggcgctggcgtcgttcatgttgacgcgggcggccttacgcactgcatccacggtcgggaagttctcccggtcgccttgctcgaacagctcgtccgcagccgcaaaaatgcggtcgcgcgtctctttgttcagttccatgttggctccggtaattggtaagaataataatactcttacctaccttatcagcgcaagagtttagctgaacagttctcgacttaacggcaggttttttagcggctgaagggcaggcaaaaaaagccccgcacggtcggcgggggcaaagggtcagcgggaaggggattagcgggcgtcgggcttcttcatgcgtcggggccgcgcttcttgggatggagcacgacgaagcgcgcacgcgcatcgtcctcggccctatcggcccgcgtcgcggtcaggaacttgtcgcgcgctaggtcctccctggtgggcaccaggggcatgaactcggcctgctcgatgtaggtccactccatgaccgcatcgcagtcgaggccgcgttccttcaccgtctcttgcaggtcgcggtacgcccgctcgttgagcggctggtaacgggccaattggtcgtaaatggctgtcggccatgagcggcctttcctgttgagccagcagccgacgacgaagccggcaatgcaggcccctggcacaaccaggccgacgccgggggcaggggatggcagcagctcgccaaccaggaaccccgccgcgatgatgccgatgccggtcaaccagcccttgaaactatccggccccgaaacacccctgcgcattgcctggatgctgcgccggatagcttgcaacatcaggagccgtttcttttgttcgtcagtcatggtccgccctcaccagttgttcgtatcggtgtcggacgaactgaaatcgcaagagctgccggtatcggtccagccgctgtccgtgtcgctgctgccgaagcacggcgaggggtccgcgaacgccgcagacggcgtatccggccgcagcgcatcgcccagcatggccccggtcagcgagccgccggccaggtagcccagcatggtgctgttggtcgccgccggccaccagggccgacgtgacgaaatcgccgtcattccctctggattgttcgctgctcggcggggcagtgcgccgcgccggcggcgtcgtggatggctcgggttggctggcctgcgacggccggcgaaaggtgcgcagcagctcgttatcgaccggctgcggcgtcggggccgccgccttgcgctgcggtcggtgttccttcttcggctcgcgcagcttgaacagcatgatcgcggaaaccagcagcaacgccgcgcctacgcctcccgcgatgtagaacagcatcggattcattcttcggtcctccttgtagcggaaccgttgtctgtgcggcgcgggtggcccgcgccgctgtctttggggatcagccctcgatgagcgcgaccagtttcacgtcggcaaggttcgcctcgaactcctggccgtcgtcctcgtacttcaaccaggcatagccttccgccggcggccgacggttgaggataaggcgggcagggcgctcgtcgtgctcgacctggacgatggcctttttcagcttgtccgggtccggctccttcgcgcccttttccttggcgtccttaccgtcctggtcgccgtcctcgccgtcctggccgtcgccggcctccgcgtcacgctcggcatcagtctggccgttgaaggcatcgacggtgttgggatcgcggcccttctcgtccaggaactcgcgcagcagcttgaccgtgccgcgcgtgatttcctgggtgtcgtcgtcaagccacgcctcgacttcctccgggcgcttcttgaaggccgtcaccagctcgttcaccacggtcacgtcgcgcacgcggccggtgttgaacgcatcggcgatcttctccggcaggtccagcagcgtgacgtgctgggtgatgaacgccggcgacttgccgatttccttggcgatatcgcctttcttcttgcccttcgccagctcgcggccaatgaagtcggcaatttcgcgcggggtcagctcgttgcgttgcaggttctcgataacctggtcggcttcgttgtagtcgttgtcgatgaacgccgggatggacttcttgccggcccacttcgagccacggtagcggcgggcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgctgaa

SEQ ID NO: 10 - DNA, 53081 bp, Artificial Sequence
  plasmid
ttcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgccgcgatgaggaagcgggtgccccgcttcttcatcttcgcgcctcgggcctcgaggccgcctacctgggcgaaaacatcggtgtttgtggcattcatacggactcctgttgggccagctcgcgcacgggctggcgggtcagcttggcttgaagatcgccacgcattgcggcgatctgcttctcggcatccttgcgcttctgcacgccttcctgctggatgcgaataacgtcctcgacggtcttgatgagcgtcgtctgaacctgcttgagcgtgtccacgtcgatcaccaggcgttggttctccttcgccgtctcgacggacgtgcgatgcagcagggccgcattgcgcttcatcaggtcgttggtggtgtcgtcgatggccgtggccagttcgacggcgttcttctgctcgttgaggctcaaggccagcatgaattgccgcttccacgccggcacggtgatttcgcggatggtgtggaatttatcgaccagcatctggttgttggcctggatcatgcggatggtcggcaggctctgcatggccgaatgttgcaaggcgatcaggtcgccgatgcgcttgtccaggttggcaaccatcgcatcgaggtcggccagctcctgcacgcggcccgggtcgttcccgacattgccgcgcagaccctcggcctgctcgcgcagctcggcaaggcggaccttgccggccgcgatgtggacgccaagaaggcggtgttcctcgcgcacggctgcgaacatttcgtcgagcgaggcattgcgctgcgcgatgccttgctgggtggtctgcacttcgctgaccaggtgttcgatctgctcgcgggtcgtgtcgaagcgcgccatgaagcccgtcgaacggacgcggaagcggtcgatcagcgggccaatcaggggcaggcgggaacggttgtcggacaaagggccgacgttcagggaacgggccttggcgacaacctgggtcagtttctcgcctgcttcgtccaggtcgctgttgcgcacctggtccagcaggctatcggcgtagcgggacgtgtgctcggccacgtcgcggccgaactcggcaacggtctgcggactgccgacctcgatccgctgcgcgaccgcatggacttccggcacgtcgctttcctgcaagcccagctcgcgcagggttgccggggtcatgtcgaaggcgacgataggggccttggcgtcgtgcgtcgttttcagtgcgttcatagggttctcccgccgtgttattggttgatgccttccaggctctgcgaaaggctccgcatgagcgcctggtgagctttggccgcctcggcgaccattgccggattcatgttcttggtggtgatgagcgcgagggtgtgctgacgccagacgggcaccaggacggatgccgtttcagagaagcggtccagcatgtccacggcctgcgcccgcgtgagcttcatctgagtgacgctcatttcatgggacgccatgagggttgccaggttggcgagcttgcgcgcgaagcgttcgcgcggcttgtcgaactcgatcacgccggccttggccgcgccggcctcggggttctcgtccaggaactcgcgcccggcttgaatgtaggctctgagccggtctacctcggcctcatgcgtattgagcatgtcatccaaggcgcgcaacgtgtcccgcacgcgctgcgctacgccctcggcttcgtccagcaactggtcgagcgtcttgcgggcgacctgatacctcacctggcgttcaacctcacggccaagcatcttctcgaaccaggtaggcttttccgcgatcttgcgggggtccgcgtcggccagcttcgccacgatctggctgattttgtcggccagcgcggcaactgcgccgtgctccatcagattcgacagctcgttgagggaatccgccccgtcgatgccggccccgtactcgccaatcgtcgccggcgacgcgaagagggcgggcaaaacctcccccttcaatcgcgccatgttcacgctttgttcttccattcgatacaccctcgcggtgggttaattgcttttcgatggaagaagtttagctaaactttctatccctcgtcaacacctttagccgctaaaatttggggacaggtcatttacagaaagccagctcactcctggcgttgccccttgagcgccgctaggcgcgcagcatccttcgcgctgagaaagaacgtcatcagcggcccgaccgtcttgcttgaaccgtcggcaaagcaaacatccatcgaacagccttgcgtgtgggggtccacgccttcgaccagtttccaagggtccatgccccagccctcggcctccggattgaaccagtacgccgatgcgtcgccgtttaggtcgctgtcggcgtagtccttgaccagcacggccacccgcccggtcgtcgggcacacgtagcccggctgcttaggttcctgtcttggcattgctcaaagctccttgaaggggccgctctacagccccttgggcttgtagagcgacacgaaataggtgagtgcggtcagtaccgcgaaatgcaccaggaacgtccagccggcatgaacgccaagggtgttccagtggtacagcatccgcaggaactgaaagaaaacgtcgatagagatgatccacttcgccaccggccacaccaggacagtaacgacccatacaaagcggaccagggcctggacacccttggcaaaagtgaaccggggcggggccttgctcggggcctcaacgcgcggggcaggggcctcggcctccacttccacgcctgggaacttgataatcttcgacattgcttgaccctccacggcgatgcgtgttcaattcgtccagcgctcgcgcgcctagaccgtgatgtgacagcatcgaggtcaagcgccccggagaaatccggggcgtcatccctatgccccgtccaactcgggaaccggcttttccctggtgctcaacctggccggctcgacccacttggtgacttgctgccactcgttaccactgcgaacggctacccgaatctgcacacgtttagccgcgatcttcgtcactatgccagcaacacaaacggaatacccgtacccgccgcgcggggtgtgctgccagttcaccctatctcctacttgcatcatcatcccctggcgtcagtgaccggcccggaatttcgccaagtcgatttcgttgaaggtccagcgctgtttcgcccgttcctccaacctcgacgactcccgcatgacctcgatgcgcaggcgctcgacctggtgcatcagctcatcagcgcgccgctgcttctcggcaatagcattccgctgcgcgaccagctcctggtctacgttcggcagctcgtcgatccacggcatgaacttatcggtcatcggattggcctccggtaattgacctgggaatctacccggcctcaaaacaagaatagggcataatgccctaacttgtcaagcaattttagctaaacaattgaggggattcagcgaggcgtcatgcttgaaaacacctttcccctggcgtgcaatcagcttgtcccggcggcagcgcactgccgcagcgcggccagcaaggtgccttgctcgatccgctgtcgttcctccggcgtaatgcaatcgccgcagataccgcccagctcttcgcgcagggcgtccgccccgatcaggctgcggctaagggtcgaaatggacttaccgcagcggcggcaattggtggtgacaatctcgatcttgcggttgggcatggccctatctccttgagagaggcccgaccgtagccgggcctcgttccgttaccagcctgcgcgagccttcgcgcgttcgacttcctgggtggaccagtggccctttgcttcaccctccagggtgcaaccttgcaggtacgccttgtgccgcagctcttcggcctggcgggtaagctctgccgcctgctccatcagcgccgcgatctggtcacgacgggccagaaaatccgtggcggccgcgccgggcagctcatcgagccaagacatgatcttgcttttcatcggggttgatcctccggttgctgacctgggcgaagtgcccggccttggattgctatattagggcattatgccctagatagtcaaggaaatttagctaaacaatttgcggcgggcgcacgaaaaaacccggcttgcgggccgggctgctggcagcatatcgcaacgatcagggttggcggttttagccgctaaagtcctctcccttggcgtaaagtcctgcgggcgtcagccctggcctttccagatcgccccaatccccgctagatcgcaaaggatcgcccaggcggcataggggatcggcgaatcctcgccaacccaacgccgcaccgtgcggtcgcccttggcacccaagcccaagatgcgcgcagcctgtccgccggtgaggccggccaagtgcaagacttcccggatttctgcgccggtcggctgcacccagcgttccgccgggcgcaggcactcaagccggatattcacgtcgctcatgctgcttttctcctaatcgttatcaaatggcggcccgcaattggtcgtagccgtagcacgactcgatgcaacgcgggtcatattcacaaacctttctaccgttgcgattgatgcgatagcgcggcggcttgtcgtcgtgcgcccacacctgtcgcgtttcgggactgatcttcgtaatgatgacgtgcttcccgatttccttggcaaaaaccgggtgatgcacgctcacaatctcgcaccgcaggccaggaaagaacgtcgccggccacgccggcgcgtcctcgtccttggccggctccggctccggcttggccggtgtaaccggctccctgcgctgcccggccggctcctgagctttcgcacgcgcggccgcgagcttcgccttggcgctggccgcccatttgcgggcgatgaatgcctggtgcgccggcagcacggcatcaacccaggcgaagcccggcggcagttcgtccggcttccgccagtcctcgaccacggcccgcacgcggcgcggcggtggcgtgatacccgcgagccacgccggcagtggctcgtcggcaaggcggtcctggtcgcgggcgtccagataccggcgatgcgagcgcaacagggcgcgcaaaaccccgtccgctacctggtccaccgtcatgccgccgcgcgcatggtcgtaatgggagacgtaattgagcatttccaggcgcttgcgcctggtcaacgaaagagtcagcgccgtaggcgctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcatcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgtggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttccaacgcgagccattcgcgagagccttgagtccacgctagatctatctcatctgcgcaaggcagaacgtgaagacggccgccctggacctcgcccgcgagcgccaggcgcacgaggccggcgcgcggacccgcgccacggcccacgagcggacgccgcagcaggagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttccttgtaggcatcgggattggcggatcaaacactaaagctactggaacgagcagaagtcctccggccgccagttgccaggccgtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgacccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccactcttcattaaccgctatatcgaaaattgcttgcggcttgttagaattgccatgacgtacctcggtgtcacgggtaagattaccgataaactggaactgattatggctcatatcgaaagtctccttgagaaaggagactctagtttagctaaacattggttccgctgtcaagaactttagcggctaaaattttgcgggccgcgaccaaaggtgcgaggggcggcttccgctgtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacgccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgccggcggcatttcgagagcctgggaacgatgtacgagggctatttcgtcctcaccttgacctggttcccgccgctgctcgcccagcgcaagttcgtcgagctgatgtttgacgacgacgcgaccgcaccggatcgcaaggcgcgcacgcggggcctcatcgaccaattcaagcgtgacgtgcgcagcatcgagtcgcgcctgtcgtcggccgtgtcgctcactcgcttgaaggggcacaagatcgtcaacgaggacggcacgaccgtcacgcatgacgacttcctgcgctggctgcaattctgcgtgacgggcctgcaccatccggtgcagctccccagcaacccgatgtacctggacgccctggtcggcggacaggaaatgtggggcggggtagtgcccaaggtcggccgcaagttcgtccaggtggtcgctctcgaaggcttccccttggagtcctatcccggcatcctgacggcgctcggcgagctgccctgcgagtatcggtggtcgagccggttcatcttcatggaccagcacgaagccgtgaagcacctcgacaagttccgcaagaagtggcggcagaagattcgcggcttcttcgaccaggtgttcaacacgaacaccggcccggtcgatcaggacgcgctttcgatggtggccgatgctgaggcggccattgccgaagtcaacagcggcatcgtggccgtgggctactacaccagcgtcgtcgtgctgatggatgaggaccgcacgcgcctggaagctgcggcccgcgatgttgaaaaggccgtcaaccggttgggctttgccgcgcgcatcgagtccatcaacaccctggacgccttccttggtagtttgccgggccacggcgtggaaaacgtccgccggccgctcatcaacacgatgaacctggccgacctgctgccgaccagcaccatctggaccggcaacgcgaacgcgccatgcccgatgtacccgccgctgtcgccggcgctcatgcactgcgtcacgcaaggatcaacgccgttccggctgaacctgcacgtgcgcgacctcggccacacctttatgttcgggccgaccggcgcaggtaaatcgacgcacctggcgatcctcgccgcgcagctccgtcgctatgccggcatgtcgatcttcgcctttgacaagggcatgtcgatgtacccgctggccgccggcatccgtgcggccacgaagggcaccagcggcctgcacttcaccgtggcggccgacgacgaacgcctggcgttctgcccgttgcagttcctgagcaccaagggcgaccgtgcttgggcgatggagtggatcgacaccatcctggcgttgaacggcgtcgaaacgaccccggcccagcgcaacgaaatcggcaacgcgatcatgagcatgcacgccagcggcgcgcgcacgctctccgagttcagcgtgacgattcaggatgaggcgatccgcgaggcgatccgccagtacaccgtcgatggcgcaatgggccatctgctcgacgccgaagaggacggcttggcgctgtccgactttacagtgttcgagatcgaagagctgatgaacctcggcgagaaattcgccctgcctgtgttgctctacctgttccgccgtatcgagcgcgccctgacgggccagccggccgtcatcatcctggacgaagcctggttgatgctcggccacccggcattccgcgcgaagatcagggaatggctcaaggtgctgcgtaaggccaactgccttgtgctgatggcaacgcagagcctgtccgacgccgccaacagcggcatcctggacgtgatcgtggaatcgaccgcgaccaagattttcctgccgaatatttacgccagggatgaggacacggcggccctgtaccgccgcatgggcctgaacgctcgccagatcgagattctggcccaggccgttcccaagcgtcagtactactacgtgtcggaaaacggccgccgtctctacgacctggcacttggcccgctcgcgctcgcgttcgtcggcgcatccgacaaggaatccgtcgccatcatcaagaacctggaagccaagttcggcgaccagtgggtggatgaatggctgcgtggccggggcctcgcccttgatgaatacctggaggcagcatgagttttgcagacacgatcaagggcttgatcttcaagaagaagcccgcaacggccgcagcagcggcgacgccggccgcgaccggcccgcaaaccgacaacccgtacctgacggcgcggcgcacctggaacgaccacgttggttccgttgtgtcgcaaaagcagacctggcaggttgtcggcatcctttcgctgatgatcgtcctcgcggcggtcggcggcatcatccacatcggcagccagtcgaagttcgtgccctatgtctacgaggtagacaagctcgggcagacggccgccgtggggccgatgaccagggcgtcgaaagccgatccgcgtgtcattcacgcctcggtggctgagttcgtcggcgatgctcgcctggtgacgccggacgtagctttgcagcgcaaggccgtctaccgcctctatgccaagctcgggccgaatgacccggccaccgccaagatgaacgaatggctcaacggcaccgccgacgccagcccgttcgctcgcgcggccgtcgaaacggtcagcaccgaaatcacttccgtaatcccgcagacgcccgacacctggcaggtcgattgggtcgagacgacgcgcgacaggcaaggcgtggtgaaaggccagcccgtgcgcatgcgggccttggtgacggtctacgtcgtcgagccgacggcggacaccaaggaagaacaactgcgaaacaacccggccgggatctacgtccgggacttctcctggtcgagacttctgtgaggcactgaattatgaaaaaggaactgtttgctttggtcctggccgcgtccgttagcgtgcctgcatttgccgccgatcccggcgcggacctgactgacctctatttttccggcaagaacccggagctgaccgcgcaagagcgggcggccatcgccatcgccaagaagtgggaggcgggtaccgccggcatgcggccggtggccggccccggtggttcggtgcgcttcctgttcggcgcgcagcagccgagcatcgtatgcgccgtgctgcaagtgtgcgacgtggccctgcaacccggcgagcaagtcaactcgatcaacctgggcgacaccgcccgttggacggtcgagccggccattaccggcagcggcgcgaacgaaacccagcacctcatcatcaagccgatggatgtgggcctggaaaccagcctggtcgtgaccacggaccgccgcagctaccacatgcgcctgcgctcgcatcgcacgcagtacatgccgcaggtgtcgttcacctacccggaagatgcccttgcgaagtgggacgccatcaagaaccgcgaacagcgggatcgcgtcgagaaaaccattccgcagaccggcgagtacctgggcaacctgagcttcaactactccgtcagcgggtccacgtcgtggaagccggtgcgcgtctacaacgacggcaagaaaaccatcatccagatgccgcactcgatggaacagaccgaagcgccgacgctcctggtcgttcgcagggagggcggcctgttctccgacgatgaaacggtgatggtcaactaccgggtccagggcgaccgctacatcgtcgatacgattttcgacaaggccatcctcatcgcgggcgtgggcagcagccaggaccgcgtgaccatttcaagggggaactaaaccatgcgtaagattctgaccgtcatcgcactcgcggccacgttggccggctgcgcgacctccaagtacggcagcttcgtccaggacgcgccggccgcctacaaccagaccattgcgaccgacgcggtgaagcagctcgtcaagctctacccgccggcgcaaaccaagctggaattgcagcaggctacgcccgatccgttcggcattgccctggtcactgaccttcgcgcccagggctatgctgtcatggagtacaagcccgacggcaacgcggccgcagctccggctgctgcgtcctcggccgctgcgaagccggcaacgccgcaagcccagggcggctatccgctgcgctacgtgctggaccaattcagcgacagcaacctgtatcgcctgaccgtcatggtcggctctcaatcgctcacgcgcgcctacctcgcccaaaacaacacgatggtcccggccggcgcatgggttcggaaggagtaagccaatgagcgaagatcaaatggcaccggacgcatcgccagatgcggtcaagccgaaaagcggggttcgccgcgtcaacaacatgccgatgtacctcatcggcggtgtgctcggcatcttcctgctggtgatggccctggttgctgcggatcgcgctgcgcagcagaaccagccgggagctgcgaaggctgagaaggccggcagcaccagcatgtttgccgacgaaattgccggcaaacagcaggacggcatcatcaaggccaagccgctggagattccgccggaacaaaccgcccagcaaccgacgacggagctgacgccagccccggcgcagggaacgactatcacggtcgcacggcccgagaacctggaccagcccccgacgccgccgcagggtgcgcgcaacgaggacctggaccgcatccgcatggcgaagttgcagatgctggaagaggcgatcaaggccaagacgacggtgcgcatcgacgcgccgcgcagccagggcagcgccggcggcggtgctccgcagggccgcgaggaaacccttgcgcgcatccaggagctgcgtcggcaggctgagaacgcccgcgccaccgatccgaccgccgcctatcaggccgcgcttgcgcaggctcgcacgatgggcggcgcggcagggggtggcggtatgggcggctcgggtgcgccgaccctcgtgcagacctcgaaccgcagtggtggcggcgctggctatgggtcgttcgacaaccgcagcgagggcgaccgttggcggctcgactcccagccggaagcacctgcaacgccctatgtgctgcgcgctggcttcgtcgttccggctacgcttatctcgggcatcaactccgatctgccaggccaaatcatggcccaggtatcgcagtcggtgtacgacacggcgaccggcaagcacatgctcatcccccaaggctcgcgcctggtgggcagctactcgaacgatgtggcctacgggcagaagcgcgttctggtggcatggcagcgcatcatcttccccgacggcaaggcaatggacattggggccatgccgggcggcgatagcgctgggtatgcaggcttcaacgacaaggtcaacaaccactacttccgcaccttcgcatcggcattcctcatgtcgggcgtcgttgcgggcatcagcttgagtcaggaccgtggcaacagcaacagcggttacggacgacaagacgcgggttccgcgatgagtgaagcgttgggtcaacagctcggccaagtaacggcgcagatgatcgccaaaaacttgaatatcgcgccgacgctggaaatccgtccgggctatcgcttcaacgtcattgtcacgaaagacatgacgttttctaagccctaccaggcgtttgactattaactccaaggagtaacttatgaagaagctcgctaagaatgttttagccgctaaagtagctctggtgctggccctctcggtcggcaccttggcggtcacgcctgcgcaagcgggcattccggtcatcgacggcaccaacctgtcacaaaccactgtcaccgcgattcagcaggttgcgcaggtccagaagcaaatcgaggaataccggacgcagttgcagcagtacgaaaacatgctgcaaaacacggtggccccggccgcctacgtgtgggaccaggcgcagtccaccatcaacggcctgatgagcgccgttgataccctgaactactacaagaaccaggcgggcagcatcgacgcttacctgggcaagttcaaggacgtgtcctactacaaggggtcgccgtgcttctccctgtcgggctgctcggaaagcgagcgcaaggcgatggaagagaaccgccgcctggcgtccgaatcgcagaaaaaggccaacgatgcgctgttccgtggcctcgatcagcagcagagcaacctcaagtccgacgccgccacgctggagcaattgaagggcaaggcgacgacggcgcagggccagttggaagccctcggctacgccaaccagttcgccagccagcaggccaaccagctcatgcaaatccgtggccttctgcttgcgcagcagaacgccatcgccacgcagatgcaggcccagcaggaccggcaggcccagcaggacgctgcgggcgcgaagctgcgcgagggttcgtaccgcgcaagcccgtctaagacctggtgaggggaggcgcgatgaagaaatccaacttcatcgcagttgccgcgctggccgccgtcatggcggccagcctggcaggctgcgacaacaagcccgacaccgacaagctgacctgcgccgatctgccgaaggtcacggatgccgctcaacgcgcggagctgttgaagaagtgcccgcgcggagaaccgggaggcttcaagcccagcgaaaagaaagagtggtgatgacgtatgaaaatccagactagagctgccgcgctcgcggtcctgatgctggccttgatgccggtagcggcatacgcccaaatcgacaattcgggcatcctcgacaacgtattgcagcgctaccagaacgccgcgagcggctgggccactgtcgtccagaacgccgcaacctggctgttctggaccttgaccgtgattagcatggtctggaccttcggcatgatggcactgcgcaaggccgacattggcgagttcttcgccgagttcgtgcggttcaccatcttcaccggcttcttctggtggctgctgaccaacggcccgaatttcgcgtcgtccatctatgcgtccctgcggcagattgcaggccaggcaacggggttggggcaggggctttcgccgtccggcatcgtcgatgttggcttcgagattttcttcaaggtgatggacgaaacctcgtactggtcgccggtcgatagcttcgtcggtgcctcgttggcggccgccatcctctgcatcctggccctggtcggcgtgaatatgcttctgctcctggcgtccggatggattcttgcctacggcggtgtgttcttcctgggcttcggcggctcgcgctggacctcggacatggcgatcaactactacaagaccgtcctcggggtcgccgcgcagctcttcgcaatggtgctgctcgtaggcatcggcaagaccttcctcgatgactactacagccgcatgagcgaaggcatcaacttcaaggaacttggagtgatgctgatcgtcggcctgatcctgctcgttctggtcaacaaggtgccgcagctcatcgccggcatcatcaccggcgcgagcgtcggcggtgctggtatcggccagttcggcgctggcacgctcgtcggtgcggccgcgacggccggcgcggcaatcgcaactggcggcgcatctatcgcggccggcgctgcggcggcggccggtggcgcgcaggccatcatggcggccgcgtcgaaggccagcgataacgtctctgccggcactgacattctgtcgagcatgatgggcggcggcggtggcggcggcggtggtagcgccggcaccagcggcggcgacggcggcggctcgggtggcggcggtggctcgggcggcggtgaaaccccgatggcctcggccgccggcgacaacagcagcggcgcacgcggcggcagttcgggcggcggctcgggtggtggccgttcgtctggcggtatcggtgccacggcggccaagggcggccggatcgcggccgataccgtcgccaacctggcgaaaggtgccggctcgattgccaaggccaaggccggcgaaatgcgcgcatcggcccaggaacgcatcggcgataccgtaggcggcaagatcgcgcaggcaattcgcggcgcgggtgcggcggcgcagaccgctgcaaccgtcgccgatagcaacagccaggcgcaggaacaacctgcaccggcacccgcaccgtcgttcgacgacaacagcctttccgcaagcaacaacagggaagcggccgccgacgcggattccgaagtggcgagcttcgtcaacaagcccgcccaatcctgaaacgactcttaggagctacgaccatgcaactgaaaaaagcgttctcgtcggccgccctggtggtggccttgggcctcggcgcaactggctcggccagcgcgcaagacgtgctgacgggcgatacccgcctggcctgcgaggccattctgtgcctgtccacgggcagccggcccagcgagtgcagcccgtcgctctcgcggtacttcggcatccacaagcgcaagctgtcggacacgctcaaggcgcggctgaacttcctcaacctctgcccggtatcgaaccagacgccggaaatgcagacgctcgtttcctcgatttcgcgcggggccgggcgctgcgatgcgtcctcgctgaactccgtgctgcgtgagtggcggagctgggacgaccagttctacatcggcaaccgcctgccggactactgcgcggcctacaccggccatgcctataccgacttcaacacgaccgcgccgcgctacgtcggcacgccggaagagggcggctattggatcgaggcggccgactacgaccgcgcgctcaaggagtacgaggcgaagctgaaagagcggcagcagcagtacggtcgctatggcagcgacgcctaccgtcggttcgagcggtaaggggaggggatagcgatgccgtttgccaagctgctggcacggaacgctctgccggtggtcgccctggtggcggccactggcttcggtgcggcggatgcgaccgccgcacggctcttccccgatctgtcggaacagatggaagagcgcgttgtgtgctcggtgtctgcggccgcgaagtacgagattccggccaacattcttctcgccattcgggaaaaggagggcggcaagccgggccagtgggtcaagaacaccaatggcacctatgacgtgggcgagctgcaattcaacaccgcctacctgggcgacctggcgaagtatgggatcacggcccaggacgttgctgcggcaggctgctatccctatgacctggcggcctggcggttgcgcgggcacattcgcaacgacaggggcgatctgtggacacgcgccgctaactatcactcgcgcacgccgtcgaagaacgcgatctatcgcgccgatctgatggtgaaggccgacaagtgggcgaagtggctggatgcgcgtttcgtcaccgtcaactatggccccagctcgccggcgcagccggcagggaaggggaccacacttgcggccgctgatacgtcggcagcagcgccggccgaagcgcagccgatgaagcaaggccggatcacccgcaccagcctccgcagctcgggttacgtaccccggcagctcatcatcaacaacacgccataaggaggaacggccgtttagcggctaaagcctatgggcattcgcaacctgacgcagcgatacatgaacggggccagggcctacgcggcctgggcggcatcgcaggcgaaagcgccgtttgatcttctggtactgggcatcgggcctgtcatcgtctttggcctggtcgcgcatacgctgctcgcgttcctgcccacatgggccatgtacgccgccggcgctctgctggtcctcgcggccctgcctttggcgctgcacgtcctccgggaatacgcgctgcgctatgggcgcaaatagcgccctgcagggcgttcttactccaagggggagggcatgaatacacgcgccatgaacgacgccagcggccgggcctcgctgcctgccatggtgatcgccgacggcaccattgaagccttgaagtggctcgccttgcttcccatgaccggggatcacgtcaacaagtacctgttcaacggtacgctgccatatctgttcgaggcggggcgcttggccctgcctcttttcgttttcgtcctggcgtacaacctcgcccgcccgggcgcgctcgagcgcggtttgtacgggcgagcgatgaaacgcctgttggccttcggcctggtcgcctcggtcccgttcattgcgttgggtggagtggtgggcggatggtggccgctgaacgtcatgttcacgctgttggccgcaaccgcgatgctctacctggtcgagcgcggccgctcggtcgctcctatagcgctgttcgtcgtggccggcggcctggtcgagttctgttggccggcgctgctgctggccgcgtctgtctggttgtacctcaagcgcccgacgtgggcggccgcgttgatggcgctgctgtcttgcgcgtccctgtggtacatcaatggcaacctttgggcgcttgctgttgtgcccctggtgatcgtcgccgccggcgtcgatcttcgtgtcccgcgcctgcgctgggccttttacacgtactacccgctgcatcttgccgctctttggctgatccgcattccgatgcgcgaggcgggctacttgtttttcacctgacctttgagattccaatatgcaattgctcaagaaatgcaccatcgcggccctgccgctgctcgccctgtccggctgcgcactgctgaacatccccatgccgacgccgcccggttcgaccccgccggaaatgctgaccgtgccagtggcgcaaatctgccgcgacgctgacaagaaccctgttcgggcaacggagctgtacggcaagaaagggttgtcggccaccggcaaggtgcaggtgatttccgaaggcttcaagcctcgctatcgggtgctgctgccgcctggcagcgcctcggtccatgctgggaccgataaccagctcgccatcaagtcggtttccaccggccagaccacgcgcgtcactggcaccgtgaaggacgtgtcctacgaccataacggctgctcgatctcgcttgacgatgcgaagttctactgaggggagggcggcggatgctgacacggttgaagggcttccttgctcgtcgccgcgagttgaaggaactggatgtgtccgtggtgagccggccccggccggctccggcggaattggtccaggttgatgcacgcgaggccgtttggcgcgtgccggtgcccggccaggccgaccgcttcatgtcggccaagcctggcgcgatcaacgatgaaatgttcgtggttcgggtggacaccgaagcgttctatcgggcttggctgcgcagcagctcgacgggccgcgaaacgcggtcggacaactgcccgctgcgctcggaaatgccgcaggactacaagttcaagcacgccgtccagggcttcgcgcacggcagggaaaatcctgtgccgctggccttcgccggcgcgcaccaggagcgccaccgggtggacattggtttcagcaacggggtcacgcgctcgttctggctgattgccaacaaggctccgtcgttcccgatccaggtccacggccgggagtcggccgagctgctgaacaaggtttgcggcctcgatcctgcgccgctgtcgttcacggaactgttcgcgcaggcccaacgccaggctccgcaggtcgccacaccggcccggcctgcgccggcagcggccacccggccagctcccaaggtgcagccacgccccggccgaagcggcccgcgcaaaggccgcggactctgactacaaccgtgcgcaaggcgcattagggaggatgtatgtatgtaatcgcctgcggcatcgttgccggcttggcggctgcggtggccctgttgggcttcacgccgatgatggaggcgcttgccgccggcgaacgccgcaaggcactcgcgcaatggacgcggacgatgttcctggtgctgctgcctgtcgtgctgatgtgcgcgcccatcgggtccagcatttacgacgccgtgcaagcggacgctggcaagcccatcgctttccacaacggccggatcacggtcgtcatggccctggtcggcagcttggccgttgtcctggtcgcggctgcgcgtgcggtggtcaaccgcaagcatgccagcttctggttcgtcggctgggtgatggcgtcggttttggccggcggcgtcggcgcgatcgccagcgcgaagcaactggcgttcctcggcgaacatagcggcatggtggccttcggcttcttccgcgaccaggtgaaggacatgcactgcgatgcggacgtgatcctggcccggtgggatgaaaaggcgaactcgccggtggtctaccgctgcccgaaggcgtacctgctcaacaggttcgcatccgcgcccttcgtgccctggccggactacaccgagggggaaagcgaggatctaggtagggcgctcgcagcggccctgcgggacgcgaaaaggtgagaaaagccgggcactgcccggctttatttttgctgctgcgcgttccaggccgcccacactcgtttgacctggctcgggctgcatccgaccagcttggccgtcttggcaatgctcgatccgccggagcgaagcgtgatgatgcggtcgtgcatgccggcgtcacgtttgcggccggtgtagcggccggcggccttcgccaactggacaccctgacgttgacgctcgcgccgatcctcgtagtcgtcgcgggccatctgcaaggcgagcttcaaaagcatgtcctggacggattccagaacgattttcgccactccgttcgcctcggcggccagctccgacaggtccaccacgccaggcacggccagcttggcccctttggcccggatcgacgcaaccaggcgctcggcctcggccaacggcaagcggctgatgcggtcgatcttctccgcaacgacgacttcaccaggttgcaggtccgcgatcatgcgcagcagctcgggccggtcggcgcgtgcgccggacgccttctcgcggtagatgccggcgacgtagtacccggcggcccgcgtggccgctacaaggctctcctggcgttcaagattctgctcgtccgtactggcgcgcaggtagatgcgggcgaccttcaaccttcgtccctccggttgttgctctcgcgtcgccatttccacggctcgacggcgtgcggatcggaccagaggccgacgcgcttgcctcgcgcctcctgttcgagccgcagcatttcagggtcggccgcgcggccgtggaagcgataggcccacgccatgccctggtgaaccatcgcggcgttgacgttgcgcggctgcggcggccggctggccagctccatgttgacccacacggtgcccagcgtgcggccgtaacggtcggtgtccttctcgtcgaccaggacgtgccggcggaacaccatgccggccagcgcctggcgcgcacgttcgccgaaggcttgccgcttttccggcgcgtcaatgtccaccaggcgcacgcgcaccggctgcttgtctaccagcacgtcgatggtgtcgccgtcgatgatgcgcacgacctcgccgcgcagctcggcccatgccggcgaggcaacgaccaggacggccagcgcggcagcggcgcgcagcatggcgtagcttcggcgcttcatgcgtggccccattgctgatgatcggggtacgccaggtgcagcactgcatcgaaattggccttgcagtagccgtccagcgccacccgcgagccgaacgccggcgaaaggtactcgaccaggccgggccggtcgcggacctcgcgccccaggacgtggatgcgccggccgcgtgtgccgtcgggtccaggcacgaaggccagcgcctcgatgttgaagtcgatggatagaagttgtcggtagtgcttggccgccctcatcgcgtcccccttggtcaaattgggtatacccatttgggcctagtctagccggcatggcgcattacagcaatacgcaatttaaatgcgcctagcgcattttcccgaccttaatgcgcctcgcgctgtagcctcacgcccacatatgtgctaatgtggttacgtgtattttatggaggttatccaatgagccgcctgacaatcgacatgacggaccagcagcaccagagcctgaaagccctggccgccttgcagggcaagaccattaagcaatacgccctcgaacgtctgttccccggtgacgctgatgccgatcaggcatggcaggaactgaaaaccatgctggggaaccgcatcaacgatgggcttgccggcaaggtgtccaccaagagcgtcggcgaaattcttgatgaagaactcagcggggatcgcgcttgacggcctacatcctcacggctgaggccgaagccgatctacgcggcatcatccgctacacgcgccgggagtggggcgcggcgcaggtgcgccgctatatcgctaagctggaacagggcatagccaggcttgccgccggcgaaggcccgtttaaggacatgagcgaactctttcccgcgctgcggatggcccgctgcgaacaccactacgttttttgcctgccgcgtgcgggcgaacccgcgttggtcgtggcgatcctgcatgagcgcatggacctcatgacgcgacttgccgacaggctcaagggctgatttcagccgctaaaaatcgcgccactcacaacgtcctgatggcgtacttacccaaagaacagctaggagaatcatttatgctcagcacacttccacaagctcatgcaactttcttgaaccgcatccgcgatgcggtcgcttccgatgttcgcttccgcgctcttctgatcggcggctcttacgttcacggaggactcgatgagcactccgatttggatttcgacatcgttgttgaggacaactgctacgcagatgtcttgtctacacgcaaggattttgccgaggcactgcccggcttcctcaacgcgttcaccggcgaacatgtaggagaaccgcgccttctgatctgcctatatggtccgccactgctacacatcgatttgaagttttctcttgcttccgatctcgaccagcaaatcgagcggcgggcggttctgtttgctcgtgatccggcagagatcgagaagcgcattgaggcggcagcggtggcatggccaaaccgtccctccgagtggttcgaagcacgttgtcagcgccagtgatataagacggtaattcaccatttggattgtccgctccacccaacatgttgtttccttaaggttctcacaccagaaaggacatcaacatgctgagcagagaggacttttacatgataaagcaaatgcgccagcagggcgcgtacattgtcgatattgcgactcagattggttgctctgaacggacggtcagacgctacctcaaataccctgaaccgccagccagaaagacccgccacaaaatggttaagctgaaaccgtttatggattacatcgacatgcgcctggcagagaatgtctggaatagtgaggttatctttgcggagattaaggcaatgggttatacgggcggacgttccatgctgcgttactacatccagcccaaacgtaaaatgcgtccgtcaaaaagaacagttcgcttcgaaactcagcctagataccagctccagcacgactggggcgaagttgaggtggaggttgccgggcaacggtgcaaagttaactttgcggttaatacgctggggttctcccgccgcttccatgtcttcgccgcaccaaaacaggatgctgagcatacctacgaatcactggttcgcgccttccgctacttcggtggttgtgtgaaaacggtgctggttgataaccagaaggctgcggtgctgaagaataacaacgggaaagtcgtgttcaactccggattcctgttgctggccgaccactataacttcctgccacgggcatgccgtccacgcagggccagaacaaaaggtaaggttgagcggatggtgaaatacctcaaggagaacttcttcgttcggtaccgcaggttcgacagcttcactcatgttaatcaacaactggagcaatggatagccgatgtggctgacaaacgggaacttcgccagttcaaagaaacgccggaacagcgcttcgcgctggagcaggaacatctgcagccgttaccggatacggacttcgataccagttacttcgacatccgccatgtgtcctgggacagctatatcgaggttggtggtaatcgttacagcgttcccgaagcgctgtgtggtcagccggtatcgatacgaatatcgctggatgacgagttgcggatctacagtaatgagaaactggtggcctcacatcgcctctgttcggcatcgtctggctggcagacagtgccggagcatcacgccccgctctggcagcaggtcagtcaggtggaacatcgaccactgagtgcctatgaggagctgttgtgatgcatgagctggaagtcctgctgagtcgcctgaaaatggagcatctgagttatcacgttgaaagcctgctggaacaggcagctaaaaaagagctgaactaccgggagttcctgtgcatggcgctacagcaggaatggaacggcaggcatcagcgcggtatggagtccaggctgaagcaggctcgtctgccgtgggtcaaaacgctggagcagttcgactttaccttccagccgggcatcgaccgtaaggttgtccgggaactggctggtctggcgttcgtggagcgcagcgaaaacgtgatcctgctgggacctcctggtgtcggaaaaactcatctggccatagctcttggcgtgaaagcggtggatgcgggacatcgggtactgtttatgccactggacagactgatcgcgacactgatgaaagcgaaacaggaaaaccggctggagcgtcagctgcagcaactgagttatgcccgggtgttgatcctggatgaaataggctatctgccgatgaacagagaggaagccagcctgttcttccggctactgaaccgtcgatatgaaaaagcgagcatcatactgacgtcaaacaaagggttcgcagactggggagaaatgttcggagatcacgtgctggcaacagcgatactggatcggttgctacatcactcaaccacgctgaatatcaaaggagagagttaccggttaaaagagaaacgtaaagctggagtgctgaccaaaaacacaacgccaatcagtgatgatgaaatggtgaaaagcggacagcatcagtaacgaaagtatcttagcgggcatgaaaatggcaaataacggtcaaacatcgtggcgttgacaacgtgcctggatctggctacactatgcggccaccaagctcgcccgtggagtctctacgaagcgatcggcatgctcggtttccttccgtgagcaagtgttaggacctttgctctaccgtcgcgctggaaaggaccagcgcggagtgaggcgattggaaacccttcgactgggatgaagagcgcagacctagccaccaccattgcgctgcacgatgcgttgtctgtcagggatgcttaattaaccataaggcattcaggacgtatggcagaaacgacggcagtttgccggtgccggaaggctgaaaaaagtttcagaaggccataaaggaaaacccccactatctttcctcgaactttggcgggctcgtgaaagatagtaggggcgttcacagaatacgggataagtatatatgaaaccgtaccagagattcaaccctgtgcagtgtataaatacacggcacaatcgctccgccataagcgacagcttgtggcaggtctgaagaatacttcatataacgcagtacactggagtcagttagcacccgaagagcagatccgtttctgggaagactatgaagcgggaagggcgaccactttcctggttgaaccggaaaggaagcgcacgaagcgccgtcgcggtgagcactccaccaaacccaaatgcgaaaatccgtcctggtatcgtccggagcgctataaggcgctgagcgggcagctcgggcacgcctacaaccgtctggtgaaaaaggacccggtgaccggcgagcagagcctgcgacgtccagggaggcgttcaggacgactcacaaagaaagccgggcaatgcccggctttttctgctgctacctccgtagtcgtaaggtcgttgcaggtgctcgggtgcggtacaactcgccggtcgccagctcaagcgcgatcacgtcgttgccgtcgtagttgacgatgatgctgttgggccgactgtcctcacgcttcgcagggagaggccagccttcaatcgaagccggcgcaagctcgtagtgcttcccggtttcgacgctgcgcagcgtccaggtcctgcaaccggccacgccggtcgcagaaaccacggcgagcgagccgcgaaaatcgtgcgggtacgcctcgatgttcatacgcctcctagatcgagcgcgagcgtttctgctcggccttggccgcctgttcctgggacacctcgccgatgaccttgccctggccccggctgtaggcgatttcgtagttcttgccgacaaccggcggcttctcaaagatgccccggctgtgtttcacgatcccgccttcgctgaactggtagacgttgcgcccatcgtcgtgcagcacctggccgacgtgcttgtgcgggtggacgtttttgcttgcgtccttcgcatcgctcaactggtgaatgcctttcggtagccccgcctcgggcaacaccttcatggtcagccattcgccgttcactacctggtccacctggcggctgccgttcatgacggcgatcttgacgctgccctcgggtttcatgatgacgccagggcttgccgatgtgcgtggtgccccgatctgtactttgttcatacgctctagttctccttagtaggttctcgcgcggcgttgccgctgttcttgctgctcgatgtcttgctgcttgagctgctgcaccttctgccgctggccctcgtcgagaagcaccttgccgacagcacttctcacctggcgttcaaccccgtccttgcccaggctgctgcgctcggacaggtcgttgaaatcggtgtgcttcttcatgttcgacagggcggcgagctggccatcgttcaacagcgattccttgagcttggccgtgtcggcctcgctcaactggaccttgccggccgccgcgtccgcgaggcgcttttcagcgtgcaaatggttgcggtagttctccggggtgatcggcggcagctccttcgggtaggcgttctcgcccggcgcgaagattgggaagatggccttgccgccgaccgccttggcggcctcctgtgccttcgtcctgccgggattcacgccctgggtgatctgcacctggcggtcgtcgtcgccggcgatcacaacgggcttgtccgggaatttcgcgtgcagggcctcggcaacagcctgtaggttgccggaatcgaacgcggcgacagtcgcgtgccccagcgcttcggccactgtggcggcggtggcatagccttcgccgatcaccagcgccggcgcggccgcgagcgcatccatgccaccgacgacatggaagcatccttccttgcggctgtccttggcgaagcgcttggtgccgtcctcctggatgtactgcatggtccattgcttgccgtcggcgtcgtaggccgggatgtaggttttctggccctcctggtcggtaaggacgccggcgtgcacctgtagacccttgtcgcgcaggtacggcgtcggttccgtgatgggaaccaggctttgcgcctggcggccgatgcgctgcgccgtggcttcgtgctggcgttcttgttcctcggcacgcgcggccagcttggccgccgcctcggcctgcatcttggccttctcggcggggtccagggcgtagcccttggccttccacttcatttcgacgccggtgcggttgtttttgatgtaaccggccgggtggccgtcgaggtggccgacgtagaagcccgacttctcgcccttcttgtcgccctcggtctcgatgcggtgcttcttgccgtccatgatggggtgctcgccgcctggggtgacgacgcagcccatgcttttcagggcctccgcgaactcatcttcgggggtgacggccggggattgctgggtgggcacgttgtccggcagccagcgttgcagcttgcccatgtcggcgttcggtccggcgtaccaggacttggccaccttgtcccactgcgcgccggccgccttggcaacctggcgctcgccgtagggcacggccaggtagacgcgctcctgggccgcgttggggcgctcggccgtgggttgggtaggctgggcctcggcgcgggcctctacggccgctgtagcgccctcgcgcgcccatttggcgaacggggcagggtcaacccctgccggaacgtaccaggcgcgttcctggcggtcccagcgcgctccaagggccttcacctcgtctttctccttgaacggcacgttcaagtaggcgcgctcgggcttggcgggggcttgagcggccgccggctgctcggcggcgttcatggcctgggccatttcctgctgctcgcgctcgtagtcggcgatccggcgctgtaggtcctcgtcgtgcagcatggccgtgccctcggcggccttgcgcgcctccttggcggcaacgcggtcctcgtcggtgctgttgggatcgcggcgaactcgctcttcatggatgcgggcgaacttcgcggcctgctcgtactcgttggccgtcgcgtaagcgtcgatcacggccaggcggtcggcgagcgcttcggcattggtctgcgcgtcggggccggcgaagtcggcaagccattggtggccgccccacgcatggttcgcatagacgccccaaaactccggctctcggtcgccggccggcaccacggaccgttcgccgtcgtgctcgacctcgacgttggcctggacctggacgcggccggtccaatcggcaggcagctcaaagcccagcgtggtttcggtcagcgcggccagcgattggttgccttccgccggctccgcgccggcgcggtacatgcgcagggtctgcgcgatcagctcgtcggccggcgcaatggccggtcgtgccacctggtcttgctgttgctccatagttgccccctgcgcaggctcgatggcctgctgggtcgtttgttcttgaatttgcttctgctcgaacgccaggacgaaatcctggatcttctccgcgtcggcggccgcgcggaaaatctctagcgggtcctcttgtagcgccttgatccacgatccgacataggccgcgtgctggccggggtcgtggccgatgcccagctcgtcgcccaggatcatgctggcaatctcggcccgcagctcttccttggcgtacccctcgctcccgaagggatgcgccaggtcgcggtccagccgcgacgggtggccggtccagtgccccagctcatggagcgcggttgcgtagtagttgtcggcgctcgggaactggcctttgtcgggcagatggatgctgtccgtggacggccgataaaacgcgcggtcgtgctcgccgtggcggatggtggcacctgacgccgcaaggatgtgctcggcccgctcgacggcgctccaagtctgttccttgcgttccaacggcggcaggccgtcgatctgctccgcattgaacacggtggcgaagaacacgcgcgggcgttcgagctgcaccgtcaccttgaccggatcgccgttggcatcgaggaccggcttgccggtctgctcgtcggtcttggtctgctcttcgctgaacttccaatactggatcggcgtgcctttctcgccgcgacgcacctgtgcgccggcggcagcggcctgcttgtaggtcatccagcgcgggtccgcatggccctgggccatgagctgaatcgcgttgatgcccttgtaacgcttcccggtagtcgggttgagcgggatgaaggagccgggcatgcccggttcccacggtttttgccacggcgcagtgccggctttcagttgctcaatgaggcgttcggcaacctgctcgtggaacggctttttgacctctgccatagccaattacctcccgtcattggcggccgcggtcgtcgtgtcctcgggcacggtcgcgtccaccaggtcaatgtcgctctcggcggcgtcctgctcgctcaaggcgtcctcggggaaggccccggccttctccgcttcttccgggtcgaactcgacctggaagccgggcgtcatcgcatcggcgagcttttcacgcagggcggcggcggcgttcgcggtatcgtcgaacggcgcaaagtcgtcctgctgcacggtcttgggatcattcatcgctttcactcctggttggtgccgttacggcctttgctgtagtccggcctgcctttcaggtcggggtatgtctgcttgcacgtcgggaagttgctgcacccccaccagaacatgccgcgcttcttgccaggccgacgggaaaggccgtggccgcaggccatgcacttgtgcagctcggagactttcggggcctcgcgcgggacgggcttgccgcccttgtcgtcgcacgcgaacttgcagccgtcggcaaagccggtgcagccccaaaagtattcgttcttgtccttcttcttgaggcgtcgcagcggcttgccgcaggacgggcaagggtgcgtgtcgatcttcatgttgaggccgttgtctttgatgttggcgacctcggcgccgatgtattccatcagctcgttgacgaacgacagcgtgtcgcgctcgccggcctggatggccttctgctgctcatgccagagcgcggtcatgtcggggaatctggccgtgtcgggcagtgcgtcgtacagctcttcgccggtcggcgtggacacgatgtgcttgcccttctccaccaggtagccgcgctcgaaaagcgtggcgatgatggagtctcgcgttgccggcgtgccgatcccgccgtgctcgccttgcttgcccttgtccttttcgatcaagattttccgcaggcggtcatcgcggatgtatttcgcaacgcgggtaaggtccgacagcagagattccatcgtgtacagcggctgcggtttcgtctcctgctgctcggccttcgcatcggtgcaggtgccggcctggccgtcacgcagcttgcgcaggtcctgttcaatgtcgtcggcattgccttccaggtcctcgttgccggcgtcgttcttgtagagaatcttccagcccggcgacgtggtgacgttcgagcgcacgccgaaacgatgatcgccgacctgggcaagcacgtcggtctggtcatacagatgcttcggccagaactgcgcgacgtaggcgcgcgcgatcagcaggtaaatcttctgctcggcatcggtgagcttcgacaggtcggccgtgctttcggtcgggatgatcgcgtggtgcgcggaaaccttggacgagttgaaggcgcggctcttgatcgtcggattggcgcgctgcgcagcagcggccagcatgggggccgtctgtgcgatggccgccagcacgcccggcgcatcgccgtgctgttcctcgctcaagtattcgcagtcggaacggttgtaggtgatgagcttgtgcttctcgcgcagggcctgcgtaatgtccttcacctggtccggcttgaagccgaacttgcgcgaggcgtccatttgcagtttcagcaggttgtagggcagcggcgcagccgcttccttcgccttggtggtcacggacacgatgcgggcgggttggccgctcacggcggccgcgatgccctcggcgtgctccttgttgctgaggcggcctttctcgtccaccggatcgccgtcggcgacctggtaacgggccgggaactgaatgccctcgacctcgaactggccgttcaccaggtagtagtaggttttctggtgggccgcgttctcgcggcaacggcgcacgacaaggcccaggatcggagtctgcacgcgccccacgctcaacagcccctgatagcccttcgcgcgtgccgcaagcgtgtacaggcgcgtgatgttgaagccgtatagctggtcgccgacgctgcgggcctcggccgcagcggacaggccggcgaactcgcggttgtcgcgcatcgcggcgagctgccggcgcacgatcttcacgttgttgtcgttgataagcagccgctgcaccggcagacggcagttggcgtattccaggatttcatcgaccagaagctggccttcgtcgtccgggtcgccggcgtgaaccacgcttttcgcctgcttcaacaggctgaggatggtcttgaactgagctttcgcaccggcatcgccggacggtttcttgcgccagggaatatggacgatgggcaggtcggccatgttccagttggcgtagcgctcgtcgtagtcctccgggtctagcaaggccagcatgtgaccgtagcaccaggtcacgcggtcggagccgcattcgtaatagccgtccttgcggctgccgccgcccaggccctcgacgatggcttttgccagctccggtttttcagcgattacaaggcgttcaaattgcatatatccccctaccctcaccaggtcagaaccggcctgatgacggtgatgatttgcgaacgattgacaggcccgaagtagcggccgtcgaaagacgtgtcgcttacgtcggacataagcagaacctcggcggtccccagggtgtagctgtcggactgataacgaggcagcggccgtcctgatggatcggccttgatgagcgcgctgtgaggcagcagcccgccattcacgcgcacgccggcgtcggtgatggcaacctcgtcgcctttagcggctaaaactcgcttcatcatgtagccgtagtcgccggggcagaaaccgccggcgatgtagccccgctccttggcgtccgaaaacacgccgacttgcggcgggcagaacatgacgtaagcccccttctccaccggcgcattcgatttccagtacaggccgaccggaatgcttttggtggtgttgaccttcgcgccggcgagataggccgcgccggcgagcaacaaggccgcgccgcctccgatggcgacgtacttggtgaggcgctggaagcggctcatatcgtgatcccctccccttcctcgacggtggccgtctggatcagcttgtcgctgaccttcggagccggtacggccgcgcgggcctggaatatcgggtctttgaagtagagcggctgcttgccgtagatcgcgggatagccggcgacgtacacaaccatgtcgcccgcctcttcaatgctgccgtcggcgctcttcttcggccccggcatgcgcaggcattcatcgggggtcagcaatggccgctgcacttcctggaaggtccgcgagacgttgcccaacagcgccgacgtgcggcggccgctcgtcgtgatctgctccttcacgatggtcgtggtgcctgtcagttttgacaggtgctcggccgtctccacgcggttcggcgggtaggcgttctgcacgtggcagttcgacgtgatgctttcgtcgtggccgtagccggtttcgcggctcttgagctggttaatgtcctggcagatgaggtagcacttgatgccgtagccggcgacgaaggcaagggactcttgcaggatttcgagcttgcccaggctggggaactcgtcgagcatcatcagcagacgatgcttgtagtgcgcgacaggacggccgttctcgaagtccatcttgtcggccagcagccggacgatcatgttgaccatgacgcgcaccagaggccgcagacgggccttgtcgttgggctgcgtcacgatgaacaggcttaccgggtcgtcgtggtgcatcagttgcttgatgcggaagtcggacttgctgacgttgcgggccacaaccgggtcgcggtacagggccaggtaggacttggcggtggacagcacggaaccggattcttcctccgggcggtccatcatgtcgcgggccgcagagccgaccgcagggtggttctgcccgtcaacgtggccgtaggtggtcatttccatccaaagctcgcccacgtcgcggttcgggtcggcaagcatgccgtccaccgacggcagggtggccggcgtaccctcgttcttagccttgtagagcgcgtgcaggatgacgccgacaagcagcgcctggctggttttctgccagtgcgattccaggcccttgccgtccggatcgacgatcagggtggcaaggttctgcacgtcgccaacctcgtactcggtccccaagcggatttcatcgagcgggttccagcacgcgctaccctgcgcggatgccggctcaaagcgcacgaccttgttgcgggcatgcttcttccgccagccggcggtcagcgcccacaactcgcctttcaggtcggtgatgacggcgctgtgcgcccaggaaagcagcgtcggaacgaccaggccgacgcccttgccggagcgcgtcggcgcgtaggtcaagacgtgctcggggccgttgtgccgcaggtagtggaacttgccgtccttgtcctgccagccgcccacatagacgccgctggaagtgggcgggtgtttgcctgacaccagctcgacgacggtgcgcggccggggcagcaggccggcggcctgtatgtccttcttgtcggcccagcgggccgaaccgtgcagatagtcgttcgccttgccggtgttcgccttgaccatctgcgtgacggccgtgcccagcaggcccacggtcgaaacgaccatacccatgctggccgcgcgcatgaaatcgtcgggatattggccgtaccacttgccggcccattgaaggatcgaccagggcgtgtagacgtggttgatattccagccaagtccggcctgatactggaaggaatgggcgaaatattgcgtcgcggtctgcaagcctgccccaagggacaggccggcgaggatgggaacggtcttgctggccttcggttttttcgcccgtatctgtggccccacggcgttgtttcggttcttcatctactcctacctcgggtagttttaagggagcctcgcggggtcacggtgacgggatcaccgatggcgaggcgcttcatgcgttgcaccgtggccttatcgacgggcagcaccagaatctcgtcgttttctttcctcaacagggccagcgcctggtcctcgacgttccgggtgcctgcataggacagcgcaccaacataatcagtatatcgtgcatgcttcggtatatcgaagccgtttagccgcttttgctcgcgctcggcaacatatttctcggccgccgcgatctgttcgggctttagccctcttcctggcccagaaactccccgtcgcagtgcgtgagctggttcggctccttgctgctccacgtgaccaggaacatcacgcggcaatagcatttcagctccgccggcgatgcgaaccacaccgagttgggacagcgctcgcaaacggttttggctttggggcggcggctttcgtccaatgcgtccaacgttgggcttgcggagtgcgacggttccgccggcgctgacggcgcgagcgtcccgtcggtcgccgtcgccgcctgtggcgttgagggtggttctggctgcggcaggtcgaatgcctccatcgccgccgcgatctcttcgtccgtcatttcgttcgggttgctcatgtgcttgctccttcgtcagtagttcttgacggcggcgctcaagggcggcgtcgtcaaaggtgattgccagacggccagcggcggccgcctgcgcgatccgctccttgaactctgctgtgccgttgacggtgatccggtcgccgaagcgctccattgccaggcgcagggcggcgtccaggccgtccgtggtggcctcgcgcgagacttgcaggcggtcgccgtcgtcgcggacggcgctgctgccgacgcgatagatgatggttcccttcttcgtgatgttgtccgtcacggccgcatggcccggcttggcctcgccgctgccctggatggtgttgcccttgaggtcgctgcggccctcgcgtgcgcgcagcgcggccagggccttgtcgtcgcccttcatcgcctcggccttgagccagtcggcccacgcgcggcgctgcgtgcgctcctggaccgcctgacggccctgccggtactcgcggttgatcttgtccaggtcggcgcgcagagccttgtgcgcctgcgcgtacatcagtcgctttgcaatgcgcccctcgcccagcagcttgatagcggcgcggcgcagccggttgctgcgcatcgcggcttcaatcaggcggtcacgacgccggcgcagcgtgtccagctcgcccttgcgcacggcccccatttcctggcgttcagactgataccgggcgtatagctcggtggtgtcgatgcgggtcttgagcggcttcgctcgatactcccgccgccggggggcttcgccgccctcggctggcgtgaatgccccgaatcgggcttcgagcttcggcttggacaggtcgcgcgaaacggtgctggccttgaccgtcgtgccgtcgccggcctcgaagatgaagccgtttccgcgctcgcgcagcttaagcccgttttcccgcaggacgcggtgcaggtcctcccaggattgcgccgcttgcagctccggcaggcattcgcgcttgatccagccgaccaggctttccacgcccgcgtgccgctccatgtcgttcgcgcggttctcggaaacgcgctgccgcgtttcgtgattgtcacgctcaagcccgtagtcccgttcgagcgtcgcgcagaggtcagcgagggcgcggtaggcccgatacggctcatggatggtgtttcgggtcgggtgaatcttgttgatggcgatatggatgtgcaggttgtcggtgtcgtgatgcacggcactgacgcgctgatgctcggcgaagccaagcccagcgcagatgcggtcctcaatcgcgcgcaacgtctccgcgtcgggcttctctcccgcgcggaagctaaccagcaggtgataggtcttgtcggcctcggaacgggtgttgccgtgctgggtcgccatcacctcggccatgacagcgggcagggtgtttgcctcgcagttcgtgacgcgcacgtgacccaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggcttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcattgggtgactcctgacggttgcctctggtgttcaaacgtgtcctggtcgcttgccggctaaaaaaaagccgacctcggcagttcgaggccggctttccctagagccgggcgcgtcaaggttgttccatctattttagtgaactgcgttcgatttatcagttactttcctcccgctttgtgtttcctcccactcgtttccgcgtctagccgacccctcaacatagcggcctcttcttgggctgcctttgcctcttgccgcgcttcgtcacgctcggcttgcaccgtcgtaaagcgctcggcctgcctggccgcctcttgcgccgccaacttcctttgctcctggtgggcctcggcgtcggcctgcgccttcgctttcaccgctgccaactccgtgcgcaaactctccgcttcgcgcctggtggcgtcgcgctcgccgcgaagcgcctgcatttcctggttggccgcgtccagggtcttgcggctctcttctttgaatgcgcgggcgtcctggtgagcgtagtccagctcggcgcgcagctcctgcgctcgacgctccacctcgtcggcccgctgcgtcgccagcgcggcccgctgctcggctcctgccagggcggtgcgtgcttcggccagggcttgccgctggcgtgcggccagctcggccgcctcggcggcctgctgctctagcaatgtaacgcgcgcctgggcttcttccagctcgcgggcctgcgcctcgaaggcgtcggccagctccccgcgcacggcttccaactcgttgcgctcacgatcccagccggcttgcgctgcctgcaacgattcattggcaagggcctgggcggcttgccagagggcggccacggcctggttgccggcctgctgcaccgcgtccggcacctggactgccagcggggcggcctgcgccgtgcgctggcgtcgccattcgcgcatgccggcgctggcgtcgttcatgttgacgcgggcggccttacgcactgcatccacggtcgggaagttctcccggtcgccttgctcgaacagctcgtccgcagccgcaaaaatgcggtcgcgcgtctctttgttcagttccatgttggctccggtaattggtaagaataataatactcttacctaccttatcagcgcaagagtttagctgaacagttctcgacttaacggcaggttttttagcggctgaagggcaggcaaaaaaagccccgcacggtcggcgggggcaaagggtcagcgggaaggggattagcgggcgtcgggcttcttcatgcgtcggggccgcgcttcttgggatggagcacgacgaagcgcgcacgcgcatcgtcctcggccctatcggcccgcgtcgcggtcaggaacttgtcgcgcgctaggtcctccctggtgggcaccaggggcatgaactcggcctgctcgatgtaggtccactccatgaccgcatcgcagtcgaggccgcgttccttcaccgtctcttgcaggtcgcggtacgcccgctcgttgagcggctggtaacgggccaattggtcgtaaatggctgtcggccatgagcggcctttcctgttgagccagcagccgacgacgaagccggcaatgcaggcccctggcacaaccaggccgacgccgggggcaggggatggcagcagctcgccaaccaggaaccccgccgcgatgatgccgatgccggtcaaccagcccttgaaactatccggccccgaaacacccctgcgcattgcctggatgctgcgccggatagcttgcaacatcaggagccgtttcttttgttcgtcagtcatggtccgccctcaccagttgttcgtatcggtgtcggacgaactgaaatcgcaagagctgccggtatcggtccagccgctgtccgtgtcgctgctgccgaagcacggcgaggggtccgcgaacgccgcagacggcgtatccggccgcagcgcatcgcccagcatggccccggtcagcgagccgccggccaggtagcccagcatggtgctgttggtcgccgccggccaccagggccgacgtgacgaaatcgccgtcattccctctggattgttcgctgctcggcggggcagtgcgccgcgccggcggcgtcgtggatggctcgggttggctggcctgcgacggccggcgaaaggtgcgcagcagctcgttatcgaccggctgcggcgtcggggccgccgccttgcgctgcggtcggtgttccttcttcggctcgcgcagcttgaacagcatgatcgcggaaaccagcagcaacgccgcgcctacgcctcccgcgatgtagaacagcatcggattcattcttcggtcctccttgtagcggaaccgttgtctgtgcggcgcgggtggcccgcgccgctgtctttggggatcagccctcgatgagcgcgaccagtttcacgtcggcaaggttcgcctcgaactcctggccgtcgtcctcgtacttcaaccaggcatagccttccgccggcggccgacggttgaggataaggcgggcagggcgctcgtcgtgctcgacctggacgatggcctttttcagcttgtccgggtccggctccttcgcgcccttttccttggcgtccttaccgtcctggtcgccgtcctcgccgtcctggccgtcgccggcctccgcgtcacgctcggcatcagtctggccgttgaaggcatcgacggtgttgggatcgcggcccttctcgtccaggaactcgcgcagcagcttgaccgtgccgcgcgtgatttcctgggtgtcgtcgtcaagccacgcctcgacttcctccgggcgcttcttgaaggccgtcaccagctcgttcaccacggtcacgtcgcgcacgcggccggtgttgaacgcatcggcgatcttctccggcaggtccagcagcgtgacgtgctgggtgatgaacgccggcgacttgccgatttccttggcgatatcgcctttcttcttgcccttcgccagctcgcggccaatgaagtcggcaatttcgcgcggggtcagctcgttgcgttgcaggttctcgataacctggtcggcttcgttgtagtcgttgtcgatgaacgccgggatggacttcttgccggcccacttcgagccacggtagcggcgggcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgctgaa

SEQ ID NO: 11 - DNA, 31 bp, Artificial Sequence
  primer
aggcgtcgaccaaagggttcgcagactgggg

SEQ ID NO: 12 - DNA, 50 bp, Artificial Sequence
  primer
gacgtcgctaacagatcttccttaattaaggcatccctgacagacaacgc

SEQ ID NO: 13 - DNA, 50 bp, Artificial Sequence
  primer
ttaattaaggaagatctgttagcgacgtccagggaggcgttcaggacgac

SEQ ID NO: 14 - DNA, 31 bp, Artificial Sequence
  primer
ccgcaagcttcacagccggggcatctttgag

SEQ ID NO: 15 - DNA, 28 bp, Artificial Sequence
  primer
gtcgacgtcccctgttatccctacccgg

SEQ ID NO: 16 - DNA, 29 bp, Artificial Sequence
  primer
gcgagatctagggtaatcccggatcttcg

SEQ ID NO: 17 - DNA, 25 bp, Artificial Sequence
  primer
agccatggccataaggcattcagga

SEQ ID NO: 18 - DNA, 22 bp, Artificial Sequence
  primer
gtggatccgcaggctctgctcg

SEQ ID NO: 19 - DNA, 22 bp, Artificial Sequence
  primer
atggtgacaaagagagtgcaac

SEQ ID NO: 20 - DNA, 19 bp, Artificial Sequence
  primer
ttacagcccttcggcgatg

SEQ ID NO: 21 - DNA, 35 bp, Artificial Sequence
  primer
gtccaattggtcgaccgtcacaattctcaagtcgc

SEQ ID NO: 22 - DNA, 36 bp, Artificial Sequence
  primer
gtccaattgctcgaggtcacaccatcctgcacttac

SEQ ID NO: 23 - DNA, 30 bp, Artificial Sequence
  primer
cgcgtcgactagccgtagcacgactcgatg

SEQ ID NO: 24 - DNA, 29 bp, Artificial Sequence
  primer
caattacgtctcccattacgaccatgcgc

SEQ ID NO: 25 - DNA, 33 bp, Artificial Sequence
  primer
cgtaatgggagacgtaattgagcatttccaggc

SEQ ID NO: 26 - DNA, 28 bp, Artificial Sequence
  primer
cggaagcttggcggacgttgacacttga

SEQ ID NO: 27 - DNA, 28 bp, Artificial Sequence
  primer
cgcgtcgacccgctagatcgcaaaggat

SEQ ID NO: 28 - DNA, 29 bp, Artificial Sequence
  primer
gaatcgggtatcccattacgaccatgcgc

SEQ ID NO: 29 - DNA, 30 bp, Artificial Sequence
  primer
cgtaatgggatacccgattctgcggttaca

SEQ ID NO: 30 - DNA, 32 bp, Artificial Sequence
  primer
tatgccgccggacgtaattgagcatttccagg

SEQ ID NO: 31 - DNA, 36 bp, Artificial Sequence
  primer
atgctcatccggagttccgtatggcaatgaaagacg

SEQ ID NO: 32 - DNA, 36 bp, Artificial Sequence
  primer
cgtctttcattgccatacggaactccggatgagcat

SEQ ID NO: 33 - DNA, 25 bp, Artificial Sequence
  primer
atcgaattccggccgtacccgattc

SEQ ID NO: 34 - DNA, 25 bp, Artificial Sequence
  primer
gagatagatctagcgtggactcaag

SEQ ID NO: 35 - DNA, 30 bp, Artificial Sequence
  primer
catgaattcgtttagagcgagccaggaaag

SEQ ID NO: 36 - DNA, 29 bp, Artificial Sequence
  primer
tgaagatctaccgcagggaaattctcgtc

SEQ ID NO: 37 - DNA, 32 bp, Artificial Sequence
  primer
agcttacgttaattaaatgtacgacgtcctaa

SEQ ID NO: 38 - DNA, 32 bp, Artificial Sequence
  primer
atgcaattaatttacatgctgcaggattctag

SEQ ID NO: 39 - DNA, 37 bp, Artificial Sequence
  primer
atccaattggatttaattaaccggccgtacccgattc

SEQ ID NO: 40 - DNA, 39 bp, Artificial Sequence
  primer
attctagatacgaattctacctcaaggctctcgcgaatg

SEQ ID NO: 41 - DNA, 41 bp, Artificial Sequence
  primer
atccaattggatttaattaagtttagagcgagccaggaaag

SEQ ID NO: 42 - DNA, 39 bp, Artificial Sequence; identical to SEQ ID NO: 40
  primer

SEQ ID NO: 43 - DNA, 27 bp, Artificial Sequence
  primer
atgtctagaactgtcaaagcgcacccg

SEQ ID NO: 44 - DNA, 29 bp, Artificial Sequence
  primer
atgtctagacgctgtctttggggatcagc

SEQ ID NO: 45 - DNA, 30 bp, Artificial Sequence
  primer
atgtctagaccgcagtcattgggaaatctc

SEQ ID NO: 46 - DNA, 30 bp, Artificial Sequence
  primer
atgtctagaccgtgaccaaagttttcatcg

SEQ ID NO: 47 - DNA, 30 bp, Artificial Sequence
  Primer
agtgcatgcgaagatggagatttcccaatg

SEQ ID NO: 48 - DNA, 80 bp, Artificial Sequence
  alignment
tcgccatcggccggcccctcaagtgtcagcacgccgccacgcgatcacgtcgcgggcttcgtagccgtggcgcttgcagt

SEQ ID NO: 49 - DNA, 80 bp, Artificial Sequence
  alignment
tgcctggggagcgttgttcctggtcatggtcgcctcctggaagccccggccgaagccggggccgcccctcatcagtcaat

SEQ ID NO: 50 - DNA, 160 bp, Artificial Sequence
  alignment
atggtgatcgccatcacgtttcattgacacttgaggggcgtttagagcgagccaggaaagccgaccccctccttggagtaaaaacccttgcggcgttgcagccggcacggatcttccgatcgggcgcggtggtggccgcgtctgtgacctaaaaaggggg

SEQ ID NO: 51 - DNA, 160 bp, Artificial Sequence
  alignment
acggcgcaggagtcaccgcggccttgacagttggggagtccagagggggccttggccccctttggaagagcacgaacttctgccaggcgcttgcgcctgctatggtcagccgtagcgccgtaggcgctccattcttggggtgaaggccgtaggccgaggg

SEQ ID NO: 52 - DNA, 545 bp, Artificial Sequence
  alignment
ggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcatcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgtggtatccacaacgccg

SEQ ID NO: 53 - DNA, 545 bp, Artificial Sequence
  alignment
ataacaactaggtttaatataaaggtgatcattctgcggggcaccttgggccggtttgccgcttttttcgacacccggttataacaactaggtttaatataaaggttatgaagggttaaaagccggttagcttgaccggaaaacgcgcgcaagccgttgaaaaacgcgcgcaagccgttgatacgccaaggaaaaggctcgaagggccgcccctcaagtgtcaatggataagcccctcaagggtcaatagtctgcccgtcaagtgtcaaggatcgcgcccctcatctgtcagcacccccgcccctcaagtgtcagtacggcaagggccttgccaacaggcttatccacaacttctgtggataaagccagcccgatcaagcacataggccgcctcgacggcctccggcgcggctccagccggccgatttcgaggccggcccctcatctgtcagcatcgcgccggggtgcgctgccccccaagtgtcaagaaacgcccctcaactgtcaccagccggcgttttgtccccaaacgatccacaattccg

SEQ ID NO: 54 - DNA, 450 bp, Artificial Sequence
  alignment
tcgccagcctctggttaaaactacctggttatataaaaaggttataaagacggttaaaaacctggttagcatgaccgaaaaacgcgcgtaaaccgttgtcacgccaaggaaaagcgcgaagaggccgcccctcaagtgtcaatggataagccccccatgtgtcaatacgctgcccctcaagtgtcaagaaccgcgcccctcaagtgtcagtagggccgcccctcaagtgtcaataccggggcagggttcctaacagggttatccacaacttcggtggataactccatgccgatcaggcacttaggccgcctggacggctcgaccaggccgaaatcggccgctgcccccaaagtgtcaacatcgcgccggccccgctgcccctcaagtgtcaacgtcagggctgcgccggccggcctcggccgacttgtcccgaaactgtccacaacggcc

SEQ ID NO: 55 - DNA, 246 bp, Artificial Sequence
  alignment
cgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttcca

SEQ ID NO: 56 - DNA, 246 bp, Artificial Sequence
  alignment
cctctatgccccgagaaagggccagaaaggcccgccagggcctcgtagcctagcggcgaggccaccggcccggtgagcgtcgggaaggcgctggaagcccgccaggggcgagaccggccgtggccgcctggctcgatgcgtagcacgacatagccggttctcgcggccgacatagccggttctcgcggccgccggccgggagaatcccccgcagcttggcccctcaaatgtcaaagagtggatctc

SEQ ID NO: 57 - DNA, 246 bp, Artificial Sequence
  alignment
cgcgccggtggggcagggcagggaaggccccggaaacgggccaggaaggcccgtcagggccgcaaagcccagcggcgagggccaccggcccggtgagcgtcgggaaggcgctggaagcccgccaggggcgagacgggcggctctgccgcctggctcgatgcgcagcacgacatagccggttctcgcggccgccggccgggagaatcccccgcagctcggcccctcaagtgtcaaagggtggatctc

(19) United States
(12) Patent Application Publication
(10) Pub. No.: US 2021/0032638 A1
(43) Pub. Date: Feb. 4, 2021

(54) PLASMID CURING

(71) Applicant: The University of Birmingham, Birmingham (GB)

(72) Inventor: Christopher Morton Thomas, Birmingham (GB)

(21) Appl. No.: 16/934,769

(22) Filed: Jul. 21, 2020

Related U.S. Application Data
(60) Provisional application No. 62/879,994, filed on Jul. 29, 2019.

(51) Int. Cl. C12N 15/70; C07K 14/245; A61P 31/04

(52) U.S. Cl. CPC C12N 15/70; A61P 31/04; C07K 14/245

(57) ABSTRACT

A conjugative recombinant vector is provided for displacing a target plasmid from a host cell. The vector is capable of replicating in the host cell, and is adapted to compete with and/or inhibit replication of the target plasmid. Also provided are systems, cells, compositions and kits comprising the vector. The invention finds use in the displacement of target plasmids such as those carrying antibiotic resistance genes, and in methods of treating bacterial infections.

### CROSS-REFERENCE TO RELATED APPLICATION

This patent application claims priority to U.S. Provisional Application No. 62/879,994, filed Jul. 29, 2019, the entire contents of which are hereby incorporated by reference.

### SEQUENCE LISTING

This instant application includes a Sequence Listing which has been submitted electronically in ASCII format and is hereby incorporated by reference in its entirety. Said ASCII copy created on Jul. 21, 2020, is named “214621-9003_As_Filed_Sequence_Listing.txt” and is 164,582 bytes in size.

### FIELD OF THE INVENTION

The present invention relates to a recombinant vector, a system and a method for displacing a target plasmid from a host cell. The invention also relates to the use of the recombinant vector in the treatment of bacterial infections.

### BACKGROUND TO THE INVENTION

Antibiotic resistance in bacteria is an increasingly urgent problem that is recognised as one of the key global challenges to public health. The rise of resistance is due to the selective pressure imposed by the use of antibiotics and other antimicrobial agents to control infection, combined with the genetic plasticity of the bacteria themselves. This allows resistance mechanisms to evolve and spread rapidly between bacteria. Once such resistance mechanisms exist, it is very difficult to get rid of them. A key part of the genetic arsenal possessed by bacteria are plasmids which are small (relative to the chromosome) DNA elements replicating independently within the cell. Many plasmids possess the ability to transfer between cells via specialised conjugative machinery and thus provide a powerful mechanism for bacteria to acquire advantageous genes from other strains or species elsewhere in the bacterial population. In a selective environment, the dominant plasmids tend to carry resistance genes and in clinical contexts where antimicrobial agents are used to treat infections, plasmids accumulate resistance determinants to the multiple antimicrobial agents that their hosts have been exposed to. One of the biggest clinical challenges to treatment of bacterial infections is caused by Gram-negative species resistant to carbapenems and third generation cephalosporin antibiotics. Most of these strains are resistant due to acquisition of enzymes encoded by plasmids. Thus, in situations where strains have become untreatable due to the accumulation of resistance genes on a self-transmissible plasmid, a possible way to reverse the situation might be to displace the resistance plasmids themselves. Also, selectively removing plasmids and the resistance genes they carry from an in-situ population should not cause major disruption to communities which could otherwise have major unintended consequences, as for example use of various antibiotics can eradicate normal gut flora and allow Clostridium difficile to overgrow with deadly effect in vulnerable patients.

In the laboratory, removal of plasmids, a process known as curing, has commonly involved harsh stresses such as increasing growth temperature or limiting thymine. Such extreme treatments are not feasible in living human or animal hosts, but specific interference with plasmid maintenance functions may be a viable route. For stable inheritance, plasmids have evolved a variety of mechanisms: controlled replication, active partitioning, multimer resolution and “addiction” or post-segregational killing (PSK) systems. The PSK systems rely on expression from the plasmid of both an unstable antitoxin or regulatory RNA and a stable toxin; the toxin becomes active in the cell after plasmid loss.

It has long been known that if closely related plasmids are introduced into the same cell, they tend to segregate into separate lineages as the cells divide, a phenomenon known as incompatibility, and this has been used frequently as a way of displacing plasmids from bacteria. However, engineering incompatibility is no simple task when the target plasmids carry multiple replicons and PSK systems, such as the very common F-like plasmids of Enterobacteriaceae. Nevertheless, it has previously been shown that strategically interfering with replication and blocking the PSK systems provides an efficient strategy for displacing such plasmids from a population without stress.

In previous work (described in WO2007/001682), efficient displacement of the F-like plasmids pO157, p1658/97 and pKDSC50, as well as the F′prolac was achieved in E. coli. The strategy used a high copy number vector that was compatible with the target plasmid. This vector contained segments of the three IncF replicons, FIA and FIB (encoding multiple binding sites or iterons that bind Rep protein and may “handcuff” the target plasmid) and FIC (encoding the transcriptional repressor CopB and the anti-sense RNA CopA that blocks repA translation) so that the cell would respond as if the F plasmid had replicated multiple times and shut off F replication. The vector also encoded either the antidote or repressor from the relevant plasmid-borne addiction systems to neutralise them and prevent them blocking growth and cell division of bacteria that lose the target plasmid when its replication is inhibited. Together these strategically chosen anti-replication and anti-addiction segments formed what was termed the “Anti-IncF cassette” or “Anti-F cassette” for short. This approach has since been extended to other plasmid groups, including IncP, IncI and IncK.

However, there remains a need to displace resistance plasmids from the microbiota of a complex environment such as a human or animal gut, where Enterobacteriaceae make up less than 1% of the community. In particular, there is a need to provide a conjugative vector which can be used to target a broad range of plasmids and which is transmitted through a population of bacteria by itself.

The present invention has been devised with these issues in mind.

For the avoidance of doubt, all references cited herein are incorporated herein by reference in their entirety.

### SUMMARY OF THE INVENTION

According to a first aspect there is provided a system for displacing a target plasmid from a host cell, the system comprising:

a) a conjugative recombinant vector which is capable of replicating in the host cell, wherein the vector is derived from an IncP parent plasmid comprising an IncP replicon, the IncP replicon of the parent plasmid comprising an origin of replication (oriV), or a functional fragment or variant thereof, which is associated with a series of iterons,
wherein the vector comprises an IncP replicon comprising a deletion in one or more of the iterons relative to the parent plasmid, and
wherein the vector is adapted to compete with and/or inhibit replication of the target plasmid;
b) a first nucleic acid sequence which encodes TrfA, or a homologue, functional fragment or variant thereof; and
c) a second nucleic acid sequence which encodes KorB, or a homologue, functional fragment or variant thereof.

In some embodiments the first nucleic acid sequence which encodes TrfA, or a homologue, functional fragment or variant thereof, is provided by a nucleic acid molecule which is separate to the conjugative recombinant vector. It will therefore be understood that in such systems, TrfA is expressed in trans.

Alternatively, the nucleic acid sequence which encodes TrfA, or a homologue, functional fragment or variant thereof, may be comprised within the conjugative recombinant vector. In other words, TrfA is expressed in cis.

In some embodiments the second nucleic acid sequence which encodes KorB, or a homologue, functional fragment or variant thereof, is provided by a nucleic acid molecule which is separate to the conjugative recombinant vector. It will therefore be understood that in such systems, KorB is expressed in trans.

Alternatively, the nucleic acid sequence which encodes KorB, or a homologue, functional fragment or variant thereof, may be comprised within the conjugative recombinant vector. In other words, KorB is expressed in cis.

In some embodiments all elements required for displacement of the target plasmid from the host cell, including sequences encoding TrfA and KorB, are provided in the conjugative recombinant vector.

Thus, in a second aspect there is provided a conjugative recombinant vector for displacing a target plasmid from a host cell, wherein the vector:

is capable of replicating in the host cell;

is adapted to compete with and/or inhibit replication of the target plasmid;

is derived from an IncP parent plasmid comprising an IncP replicon, the IncP replicon of the parent plasmid comprising an origin of replication (oriV), or a functional fragment or variant thereof, which is associated with a series of iterons, and

wherein the vector comprises an IncP replicon comprising a deletion in one or more of the iterons relative to the parent plasmid.

In some embodiments, the vector further comprises a first nucleic acid sequence which encodes TrfA, or a homologue, functional fragment or variant thereof.

In some embodiments, the vector further comprises a second nucleic acid sequence which encodes KorB or a homologue, functional fragment or variant thereof.

As is known in the art, a plasmid is a nucleic acid molecule within a host cell that is separate to the chromosome and can replicate independently. Plasmids are typically circular, double-stranded DNA molecules, often found in bacteria. However, examples of linear plasmids are known, and plasmids are also known to be present in archaea and eukaryotic cells.

In some embodiments, the recombinant vector comprises or consists of DNA. The first nucleic acid sequence which encodes TrfA and/or the second nucleic acid sequence which encodes KorB may also comprise or consist of DNA.

In some embodiments, the recombinant vector is circular.

Plasmids can be classified into conjugative and non-conjugative plasmids. As is known to the skilled person, conjugative plasmids contain a set of transfer (tra) genes which enable the plasmid to be transferred from one host cell to another by means of conjugation. For example, F plasmids are conjugative plasmids which encode the formation of a conjugation pilus on the host (donor) cell through which plasmids may be transferred to recipient cells, as well as transfer origin (oriT) and genes whose products activate oriT when the bridge between two bacteria is formed. In IncP plasmids, or recombinant vectors derived therefrom, the transfer genes comprise the transfer origin, oriT, and the tra and trb regions. These regions encode proteins for DNA processing and mating bridge formation. Thus, the recombinant vector of the invention may comprise oriT, tra and trb regions.

Plasmids can be categorized based on Incompatibility (Inc), which is the inability of plasmids which share similar replication and partitioning systems to be stably maintained in the same host cell line. Plasmids have been independently classified into Inc groups for three different genera. There are at least 27 Inc groups in Enterobacteriaceae, 14 in Pseudomonas and 18 in Staphylococcus. Some so-called ‘promiscuous’ plasmids can occur e.g. in both enterobacteria and Pseudomonas spp, and some plasmids have been allocated to an Inc group in both the enterobacterial and pseudomonad plasmid grouping schemes. Thus, some of the Inc groups of Pseudomonas are equivalent to those in enterobacteria, such as the Pseudomonas groups IncP-1 (IncP in enterobacteria), IncP-3 (IncA/C in enterobacteria) and IncP-4 (IncQ in enterobacteria).

The target plasmid being displaced may be any plasmid present in the host cell. The displacement of a target plasmid from a host cell is also referred to herein as “curing”. The plasmid may be an exogenous plasmid, which has been introduced into the host cell, for example, by transformation, and which then subsequently needs to be displaced therefrom. The term “exogenous plasmid” refers to a plasmid which originates from, or is developed or produced by, a cell other than the host cell, but which is then introduced into the host cell by some means.

Alternatively, the target plasmid may be an endogenous plasmid to the host cell, which needs to be displaced or cured therefrom. The term “endogenous plasmid” refers to a plasmid which originates from, or is developed or produced by, the host cell. The plasmid being displaced from the host cell will be autonomously replicating in the host cell.

In some embodiments, the target plasmid carries one or more antibiotic resistance genes. Such plasmids confer antibiotic resistance on the host cell, making those cell populations difficult to control or eradicate using antibiotics. For example, the target plasmid may carry one or more genes that confer resistance to an antibiotic such as a β-lactam, for example a carbapenem or a cephalosporin.

The target plasmid may belong to any Inc group. In some embodiments, the target plasmid belongs to an Inc group selected from IncF, IncI, and IncK.

In some embodiments, the target plasmid is an IncF plasmid.

The host cell may be a prokaryotic cell or a eukaryotic cell, or an archaea.

In some embodiments, the host cell is a prokaryotic cell, such as a bacterial cell. The bacterial cell may be gram positive or gram negative.

In some embodiments, the host cell is a gram positive bacterial cell selected from Bacillus spp, Lactobacillus spp., Lactococcus spp., Staphylococcus spp, Streptococcus spp, Listeria spp, Enterococcus spp, and Clostridium spp. In some further embodiments, the bacterial cell is Clostridium difficile.

In some embodiments, the host cell is a gram negative bacterial cell selected from Enterobacteriaceae spp, Pseudomonas spp, Moraxella spp, Helicobacter spp, Stenotrophomonas spp, Bdellovibrio spp, or Legionella spp. In some further embodiments, the bacterial cell is E. coli or Klebsiella pneumoniae.

The present teaching provides a conjugative recombinant vector for displacing a target plasmid from a host cell. As will be understood in the art, the term “recombinant” means that the vector has been constructed by combining genetic material from more than one source. In other words, the vector of the invention is artificial (i.e. it does not occur in nature).

The conjugative recombinant vector is derived from an IncP parent plasmid. By “derived from” it will be understood that the recombinant vector has been created by adding and/or deleting one or more segments of nucleic acid to or from an IncP parent plasmid. The recombinant vector may itself be considered to be an IncP plasmid.

Any suitable IncP plasmid may be used for construction of the recombinant vector of the invention. The parent plasmid may be an IncPα plasmid or an IncPβ plasmid. Examples of suitable IncP (IncP-1 in Pseudomonas) plasmids include: RK2, RP1, RP4 and R68, which are derived from a common ancestor whose sequence has been compiled (accession number BN000925.1); pUB307 (derived from RP41 by a deletion between coordinates 5464 and 12045 or between coordinates 5466 and 12047, the two possibilities being due to there being 3 identical bases at each end of the deletion); and R751 (accession number NC_001735.4). As used herein, the terms “RK2”, “RP1”, “RP4” and “R68” are interchangeable, because these plasmids are identical to one another.

In some embodiments, the recombinant vector is derived from (i.e. created from) RK2, which constitutes the parent plasmid. RK2 is one of a group of well-characterized IncP plasmids which have a copy number of 3-7 per chromosome. This is higher than many of the large conjugative plasmids that may be targets for displacement, so it was thought that segments cloned into RK2 should be active in blocking replication of target plasmids. However, a key part of this invention is the discovery that the natural copy number of RK2 is not sufficient for efficient curing and the unexpected discovery that removal of an iteron from the oriV region is required to make it an excellent vehicle for plasmid curing.

In some embodiments, the conjugative recombinant vector is derived from (i.e. created from) pUB307. Thus, in these embodiments, pUB307 is the parent plasmid for the conjugative recombinant vector. In particular, it has been found that, when the conjugative recombinant vector is derived from pUB307, the conjugative recombinant vector acts as a universal vector and can be used to target a range of Inc group plasmids. For example, Table 3 shows that conjugative recombinant vectors derived from a pUB307 plasmid were effective in displacing both IncF and IncK target plasmids.

The recombinant vector may have a copy number which is greater than that of the IncP parent plasmid from which the vector was derived. In some embodiments the recombinant vector has a copy number which is at least twice that of the IncP parent plasmid from which the vector was derived.

The recombinant vectors described herein must be capable of replicating in a host cell. Thus, the recombinant vector comprises one or more nucleic acid sequences encoding genes required for replication. The recombinant vector is designed such that it is capable of autonomously replicating in the host cell.

The recombinant vectors comprise an IncP replicon, which is modified relative to the replicon of the IncP parent plasmid from which the vector was derived through deletion of at least one iteron. The term “replicon” refers to a nucleic acid sequence that encodes the ability to replicate autonomously in one or more types or host cells. As is known by those skilled in the art, the IncP replicon (e.g. of the parent plasmid) comprises an origin of replication (oriV).

In some embodiments, the recombinant vector comprises a functional fragment or variant of the oriV sequence of the parent plasmid. By “functional fragment or variant”, it will be understood that the vector is still capable of replicating in the host cell. Fragments or variants of oriV which maintain replication function can be easily identified by the skilled person using standard techniques, for example by linkage to a DNA segment encoding a selectable marker (such as an antibiotic resistance gene) and determining whether this could replicate when provided with the trfA gene in cis or in trans.

In some embodiments, the recombinant vector comprises oriV from the IncP plasmid RK2, or a functional fragment or variant thereof. The essential or “minimal” oriV region corresponds to coordinates 12128-12581 of RK2 (accession number BN000925.1). In some embodiments, the recombinant vector comprises an oriV sequence which is at least 70%, at least 75%, at least 80%, at least 85%, at least 90%, at least 95%, at least 96%, at least 97%, at least 98%, or at least 99% identical to the minimal oriV region of RK2.

In the IncP parent plasmid, from which the recombinant vector is derived, oriV is associated with a series of repeated sequences, known as iterons. As is known by those skilled in the art, an iteron is a repeated sequence that is capable of binding a Rep protein, and which is located in the region of a plasmid that contains the replication origin (oriV). Each iteron is a single repeat of the sequence. The iterons may all have the same sequence, or there may be minor sequence variations between some of the iterons, such as a substitution in one or two bases. The iterons may be immediately adjacent one another (i.e. there are no bases separating them), or one or more iterons may be separated from the others. By “associated with”, it will be understood that the series of iterons is proximal to, spans or overlaps the oriV region. Some, but not necessarily all, of the iterons may be within the region defined as oriV, whereas other iterons within the series may be outside of or separated from oriV.

In different IncP plasmids that could be used as the parent plasmid, oriV may be associated with a series of 5, 6, 7, 8, 9, 10, 11, 12 or more iterons. In some embodiments, oriV is associated with 9 iterons.

In some embodiments, in the parent plasmid a group of five iterons may be associated with oriV which are essential for replication. These iterons (referred to herein as “essential iterons”) may be referred to as iterons 5 to 9 (i5-i9) because of their position in the series of iterons often present in IncP plasmids. Any remaining iterons in the series, which are not essential for replication, may be referred to as “non-essential iterons”. Such iterons may have a regulatory role. In some embodiments, the non-essential iterons may be within 500 base pairs of the essential iterons. For example, iteron 1 may be within 460 base pairs of the essential iterons, and/or iteron 10 may be within 490 base pairs of the essential iterons.

For any given IncP plasmid, the iterons can be easily identified due to the IncP plasmid backbone being well conserved. The start and end points of an iteron can be identified by creating an alignment of a given plasmid sequence with a consensus sequence and then counting out a number of bases that represent the normal length of the iteron, since not all iterons will have the first and the last base conserved.

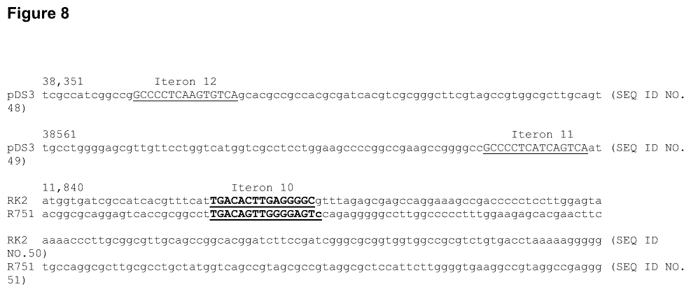

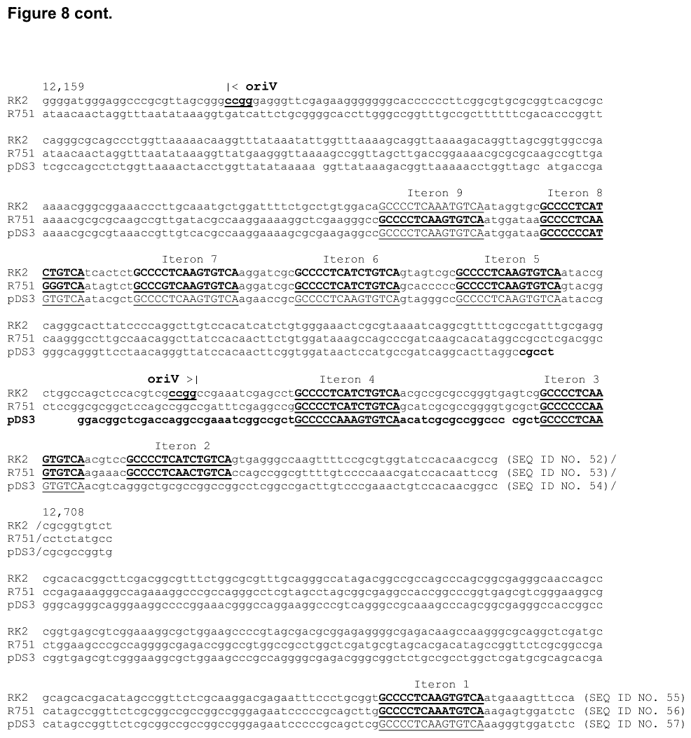

All IncP oriV regions have generally the same organization: i1 on its own; a group of two or three iterons (i2-i4); a group of five iterons (i5-i9), and a single iteron facing in the opposite direction, which is i10. All have the i2-i4 group but in some plasmids there are two iterons, rather than three. In some plasmids there may be one or two additional iterons beyond i10, as shown in FIG. 8. However, the iterons retain their numbers even if one iteron in the sequence is missing. A skilled person would be able to identify each corresponding iteron in any given system. Methods for creating sequence alignments, such as ClustalW, are well-known to the skilled person.

For example, in RK2, oriV is associated with a series 10 iterons designated iteron 1 (i1), iteron 2 (i2), iteron 3 (i3) and so on to iteron 10 (i10). Iterons 2-4 are clustered together, as are iterons 5-9, whereas i1 and i10 are spaced apart from these groups (FIG. 8). The replication origin of RK2 is described by Doran et al, J. Biol. Chem 273, 8447-8453, and Shah et al., J. Mol. Biol. 254, 608-622.

In the IncP plasmid RK2, iterons 1 to 10 correspond to the following coordinates of the annotated sequence of accession number BN000925.1:

### Iteron 1: 12928-12942 (Complement*)

### Iteron 2: 12652-12666 (Complement)

### Iteron 3: 12631-12645 (Complement)

### Iteron 4: 12596-12610 (Complement)

### Iteron 5: 12459-12473 (Complement)

### Iteron 6: 12436-12450 (Complement)

### Iteron 7: 12413-12427 (Complement)

### Iteron 8: 12391-12405 (Complement)

### Iteron 9: 12368-12382 (Complement)

### Iteron 10: 11864-11878

*It is noted that the TrfA recognition sequence in double-stranded DNA is not a symmetrical inverted repeat and was defined in the replication origin as running from iteron 1 to iteron 9 as the recognition sequence 5′-3′. The annotated IncP1 plasmid sequence in accession number BN000925.1 runs 5′-3′ in the opposite direction around the plasmid, so the recognition sequence is found on the complementary strand. The iteron 10 sequence runs in the opposite direction to the others.

In RK2, as well as the related plasmids RP1, RP2, R68 and pUB307, the sequence of iterons 1 and 10 are as follows:

Iteron 1:
(SEQ ID NO. 1)
tgacacttgaggggca
Iteron 10:
(SEQ ID NO. 2)
tgacacttgaggggcg.

As a further example, in the IncPβ plasmid R751, the sequence of iterons 1 and 10 are as follows:

Iteron 1:
(SEQ ID NO. 3)
tgacatttgaggggcc
Iteron 10:
(SEQ ID NO. 4)
tgacacttgaggggcg.

Surprisingly, the present inventors have found that a recombinant vector derived from RK2 which lacks either iteron 1 or iteron 10 of oriV enables efficient curing of a target plasmid from a host cell, whereas a vector containing both iteron 1 and iteron 10 does not potentiate curing. For example, the pUB307 plasmid is a derivative of RP1 (which is identical to RK2) which lacks iteron 10.

Thus, in some embodiments, the recombinant vector comprises an IncP replicon comprising a deletion in one or both terminal iterons of the series, relative to the parent plasmid.

It is thought that the region of oriV of RK2 which spans the coordinates 12181-12581, and which includes iterons 5-9, may be essential for plasmid replication. Thus, in some embodiments, the recombinant vector comprises an IncP replicon comprising a deletion in an iteron other than i5-i9, relative to the parent plasmid.

In some embodiments, the recombinant vector comprises an IncP replicon comprising a deletion in iteron 1 (i1) and/or iteron 10 (i10), relative to the parent plasmid.

The replication protein TrfA is required for the initiation of plasmid DNA replication. TrfA binds to iterons associated with oriV, thereby activating oriV.

Thus, the system or recombinant vector of the invention includes a first nucleic acid sequence encoding the replication protein TrfA, or a homologue or functional fragment or variant thereof which is capable of activating oriV.

The first nucleic acid sequence encoding TrfA may be a trfA gene from an IncP plasmid. In some embodiments the trfA gene is derived from the same parent plasmid as oriV. However, it is known that cross-reactivity can occur between different IncP plasmids such that TrfA proteins from one IncP plasmid may be capable of activating oriV on a different IncP plasmid. Thus, any trfA gene may be selected, provided that it encodes a TrfA protein which is capable of activating oriV. Therefore, in some embodiments, the trfA gene is derived from a different plasmid to oriV.

The trfA gene corresponds to coordinates 16,521 to 17,378 (Open Reading Frame) of RK2. In some embodiments, the recombinant vector comprises a trfA gene having at least 60%, at least 70%, at least 80%, at least 90%, at least 92%, at least 95%, at least 96%, at least 97%, at least 98% or at least 99% homology with trfA of RK2.

The system or recombinant vector of the invention comprises a second nucleic acid sequence which encodes KorB, or a homologue or functional fragment or variant thereof. KorB is a DNA-binding protein which is involved in plasmid partitioning and is known to be a global regulator of IncP-1 plasmid gene expression. Unexpectedly, the inventors have found that the presence of the korB gene is essential for the potentiation of curing. Without wishing to be bound by theory, the requirement for KorB suggests that an important factor for plasmid curing may be the complex that the KorB protein makes with the plasmid DNA. It may be that the binding sites for TrfA and binding of KorB to the plasmid DNA need to be configured correctly to achieve a level of supercoiling or other topological feature(s) sufficient to activate key loci.

The korB gene, or a nucleic acid sequence encoding a homologue or a functional fragment or variant of the KorB protein, may be comprised within a nucleic acid molecule which is separate to the conjugative recombinant vector, such that KorB is expressed in trans. Alternatively, the conjugative recombinant vector may comprise the korB gene such that KorB is expressed in cis.

In RK2, the korB gene corresponds to coordinates 57184-58260 (complement).

The second nucleic acid sequence which encodes KorB (or the homologue, fragment or variant thereof) may comprise a sequence having at least 60%, at least 70%, at least 80%, at least 90%, at least 92%, at least 95%, at least 96%, at least 97%, at least 98% or at least 99% homology with korB of RK2.

The recombinant vector further comprises transfer genes which enable it to be transferred from one host cell to another by means of conjugation. The transfer genes may comprise oriT, tra and trb.

The recombinant vector is adapted to compete with and/or inhibit replication of the target plasmid being displaced. This may be achieved in a number of ways. For example, the recombinant vector may act in a competitive manner, positively out-competing the replication functions of the plasmid being displaced.

The recombinant vector may comprise a nucleic acid sequence which inhibits replication of the target plasmid.

In some embodiments, the nucleic acid sequence comprises all or selected parts of an origin of replication or one or more replicons from the target plasmid being displaced from the host cell. For example, if the target plasmid being displaced is an IncF plasmid, then the nucleic acid sequence which inhibits replication may comprise one or more of the IncF replicons, FIA, FIB and FIC/FIIA.

For example, the nucleic acid sequence may comprise a replication system such as repFIA (e.g. from F/pHCM1, accession no. AP001918, coordinates 49100-49500), repFIB (e.g. from pO157, accession number AP018489.1, coordinates 27686-29287), repFIC (e.g. from pO157, accession number AP018489.1, coordinates 74001-74660 and/or repFIIA (e.g. from pKDSC50 accession no. NC002638.1 coordinates 24300-25024).

Alternatively, or additionally, the recombinant vector may comprise a nucleic acid sequence encoding an inhibitor molecule, which inhibits or prevents replication of the target plasmid. The inhibitor molecule may be either RNA or protein. Hence, advantageously, as the inhibitor inhibits replication of the target plasmid, the target plasmid is thereby displaced from the host cells as the bacteria grow and divide. An example of a suitable inhibitor is the antisense RNA CopA of the IncFII replicon.

The recombinant vector may be adapted to replicate at a higher rate than the plasmid being displaced from the host cell. For example, the nucleic acid sequence may comprise an origin of replication or a replicon which is capable of replicating the nucleic acid molecule at a higher rate than the replication of the plasmid being displaced.

In some embodiments, the target plasmid is an IncF plasmid. In such embodiments, the recombinant vector may comprise a nucleic acid sequence which is adapted to inhibit replication of IncF plasmids. Such a sequence may be referred to as an “anti-F cassette”. An anti-F cassette may comprise one or more of: repFIA incC: repFIB; repFIC copAB; repFIIA copAB. In some embodiments, the anti-F cassette comprises all of: repFIA incC; repFIB; repFIC copAB; repFIIA copAB.

In some embodiments a large segment of a replicon may be used, including both Rep protein binding sites and the coding region for the Rep protein itself, for example the anti-FIB segment of the anti-F cassette. Alternatively, it is possible to use only combinations of Rep binding sites where these are known to inhibit replication, so as to avoid the positive effect of the Rep protein itself.

In some embodiments, the recombinant vector comprises the anti-F cassette from pCURE2 (Hale et al., (2010) Biotechniques, 48(3), 223-228). The anti-F cassette may comprise the sequence:

(SEQ ID NO. 5)
CAACACACACCAGACAAGAGAGCTGCGTGGTAGTTTCATGGCCTTCTTCT
CCTTGCGCAAAGCGCGGTAAGAGGCTATCCTGATGTTGTCTAAGCATGCA
GGGGCCTCGTGGGTTAATGAAAAATTAACTACGGGGCTTTTGTCCTTCTG
CCACACAACACGGTAACAAACCACCTTCACGTCATGAGGCAGAAAGCCTC
AAGCGCCGGGCACATCATAGCCCATATACCAGCACGCTGACCACATTCAC
TTTTCCTAAGCTTACATCCACAAACAGACGATAACGGCTCTCTCTTTTAT
AGGTGTAAACCTTAAACTGCATTTCACCAGTCCCTGTTCTCGTCAGCAAA
AGAGCCGTTCATTTCAATAAACCGGGCGACCTCAGCCATCCCTTCCTGAT
TTTCCGCTTTCCAGCGTTCGGCACGCAGACGACGGGCTTCATTCTGCATG
GTTGTGCTTACCAGACCGGAGATATTGACATCATATGCCTTGAGCAACTG
ATAGCTGTCGCTGTCAACTGTCACTGTAATACGCTGCTTCATAGCACACC
TCTTTTTGACATACTTCGGGTATACATATCAGTATATATTCTTATGCCGC
AAAAATCAGCGCGCAAATACGCATACTATTATCTGGCTTTTAGTAAGCCT
TATGTATTTTACCTTTCGTTATGTTAACCAATAAAAATTAAAATCTGCCT
TATAAAAACAAAGCGTAATTACCGCATTCCCGTTTCGTATGGTCTAGCAC
CACGCTGGGTTTACTGTTTGGTTGAAAGTTATATTTTTATTAAACATTGT
GCGTTAAAGCCTGGTGTGTTTTTTTAGTGGATGTTATATTTAAATATAAC
TTTTATGGAGGTGAAGAATGCATACCACCCGACTGAAGAGGGTTGGCGGC
TCAGTTATGCTGACCGTCCCACCGGCACTGCTGAATGCGCTGTCTCTGGG
CACAGATAATGAAGTTGGCATGGTCATTGATAATGGCCGGCTGATTGTTG
AGCCGTACAGACGCCCGCAATATTCACTGGCTGAGCTACTGGCACAGTGT
GATCCGAATGCTGAAATATCAGCTGAAGAACGAGAATGGCTGGATGCACC
GGCGACTGGTCAGGAGGAAATCTGACATGGAAAGAGGGGAAATCTGGCTT
GTCTCGCTGGATCGGGTACCTCTCGCACAGCGATTTTCGTGTCAGATAAG
TGAATATCAACAGTGTGAGACACACGATCAACACACACCAGACAAGGGAA
CTTCGTGGTAGTTTCATGGCCTTCTTCTCCTTGCGCAAAGCGCGGTAAGA
GGCTATCCTGATGTGGACTAGACATAGGGATGCCTCGTGGTGGTTAATGA
AAATTAACTTACTACGGGGCTATCTTCTTTCTGCCACACAACACGGCAAC
AAACCACCTTCACGTCATGAGGCAGAAAGCCTCAAGCGGCTAGAGGAGGC
TCGATCCAGTAAACAGATCCATGAATGATCAACAAAGGATCCATTAAAGA
TCCCCATACCGCTGCAAACCTTGTCACTCATGGGCCGGGACCACGATCAC
ATAAGCAGTGGCATGTTACTGATAAACTGTAACATGCTAATGATAAGCTG
TATTCAGTAATCCATATACTGAAGTAAGTTAATGACATAAACTATGGTCA
GTACGCCAGACTCAGCTGTTAAATACAGGCTGCAGGTTTTTCTTCAGTCA
GTTAGCGGGGCTCTGACACACGATTTGCTGTTTATTCTTTTACTGTCCAC
AGGCAGGAGGCTTTCTGGAAAACGAAAATTCAGACATCAAAAAACTGTTC
GGCGAGGTGGATAAGTCGTCCGGTGAGCTGGTGACACTGACACCAAACAA
TAACAACACCGTACAACCTGTGGCGCTGATGCGTCTGGGCGTTTTTGTAC
CGACCCTTAAATCACTGAAGAACAGTAAAAAAAATACACTGTCACGTACT
GATGCCACGGAAGAGCTGACACGTCTTTCCCTGGCCCGTGCTGAGGGATT
CGATAAGGTTGAGATCACCGGCCCCCGCCTGGATATGGATAACGATTTCA
AGACCTGGGTGGGGATCATTCATTCCTTTGCCCGCCATAACGTAATTGGT
GACAAAGTTGAACTGCCTTTTGTCGAGTTTGCAAAACTGTGTGGTATACC
TTCAAGCCAGTCATCACGCAGGCTGCGTGAGCGCATCAGCCCTTCCCTGA
AACGCATTGCCGGTACCGTGATCTCCTTTTCCCGCACCGATGAGAAGCAC
ACCCGGGAATACATCACCCATCTGGTACAGTCAGCCTACTACGATACTGA
ACGGGATATTGTTCAGTTACAGGCTGATCCCCGCCTTTTTGAACTGTACC
AGTTTGACAGAAAGGTCCTTCTCCAGCTTAAGGCGATTAATGCCCTGAAG
CGACGGGAGTCCGCCCAGGCACTCTACACCTTTATAGAGAGCCTGCCCCG
GGATCCGGCACCGGTATCGCTGGCGCGGCTGCGTGCACGCCTCAATCTGA
AGTCTCCTGTATTTTCCCAGAACCAGACGGTCAGACGGGCAATGGAGCAG
CTGCGCGAGATTGGATATCTTGATTACACGGAGATCCAGCGGGGGCGGAC
AAAACTCTTCTGCATTCACTACCGGCGTCCCCGGTTAAAAGCACCGAATG
ATGAGAGTAAGGAAAATCCGTTGCCACCTTCACCTGCGGAAAAAGTCAGT
CCGGAGATGGCGGAGAAGCTTGCCCTGCTTGAGAAACTGGGCATCACGCT
GGATGACCTGGAAAAACTCTTCAAATCCCGCTGAACATAAACTGTAGTCA
GTGAAGAGTGTTCCTTTACTGACTACAGCTTATATTATCAGGTGCAGTGA
GTGGTCTGCTCACTGCAGTTTATATTCAGTTTCCTGCAGTGCTGCCTGTA
GCTGAGCTGTCATCTGCCGGTCCCTTACGTGAGTCACCCCGTAACCTGAT
GCTGAGGCATTGCTCCCTTCATAAAACATGACTTACTCACTACAGCTTAT
ATACATGCTCCAGCTTATGTTATGTCTGTTCTGCTGACCACAGCTTGTCG
AGCGGATAGCCAATTCAGAGTAATAAACTGTGATAATCAACCCTCATCAA
TGATGACGAACTAACCCCCGATATCAGGTCACATGACGAAGGGAAAGAGA
AGGAAATCAACTGTGACAAACTGCCCTCAAATTTGGCTTCCTTAAAAATT
ACAGTTCAAAAAGTATGAGAAAATCCATGCAGGCTGAAGGAAACAGCAAA
ACTGTGACAAATTACCCTCAGTAGGTCAGAACAAATGTGACGAACCACCC
TCAAATCTGTGACAGATAACCCTCAGACTATCCTGTCGTCATGGAAGTGA
TATCGCGGAAGGAAAATACGATATGAGTCGTCTGGCGGATCCCCTGTCGG
TAATGAGTGAACAGTGTTCGGGTGTCAGTCACTTCTGCCTGCACTTACAA
TGCACGGAAGGAGTAAATGCCACAGAAACAAGTATTGAAATTTTCTGAGC
ATAAAACTATACTCCCTGCATAGCCTGATTGGTGGCTATACAGTTTAAGT
AGGCCCCGTTAATCTTTCGTTCCCGCCAAGAAATGAGAAGATTATCGGGG
TTTTTGCTTTTCTGGTCCCGGGTAGGCACCTCATCAGAACCAGTTCCCTG
CCACCTTACGGCGTGGCCAGCCATCAAAATTCTTTAGACGATCAGCAATC
TAACACTCACTGACGAGACGATCAAGAAAGTTACTCATTCACCTTCTTAT
CTCAGGCTCTTTTAGCCATTTCCCGTTCAATTAGTAGCTCAATCATTTGA
GCCTGAGTAGTTCCAGTCTCTTCACAAAGCTGCTGCAAGCTTTCTTTATG
TATGTTTTGTATGAAAACATTGAGCTCTTTATGCGTCGTCTTTTTTCGTG
AAATAGATAATCTCTTCTTCTCAGCGGAAGATAAGGGATTACCCTTTCTG
TAAATGCGTTTCGATGAGGAAGTTACTGCATTTTCAATCTGCGACATCTC
TGTCTCCTCAAGGATCAAACCTAGATCGTTTGCAGAGAATACTACAGATT
TCTAAAATCAGCGACTTACTTTATGAACGTAACCTGTGTTGGCGCACGGT
TTACGTTTCAAGATCGTTTCCTGGGATGTCAGTACGTCTGTTTCCAGTCC
GTTCCCTCGACTGAAGATCAGTCACACCATCCTGCACTTACAATGCGCAG
AAGGAGCGAGCACAGAAAGAAGTCTTGAACTTTTCCGAGCATATAACTAT
ACTCCCCGCATAGCTGAATTGTTGGCTATACGGTTTAAGTGGGCCCCGGT
AATCTTCTCAGTCGCCAAACTTTCTGAAGATTATCGGGGTTTTTGCTTTT
CTGGCTCCTGTAAATCCACATCAGAACCAGTTCCCTGCCACCTTACGGCG
TGGCCAGCCACAAAATTCCTTAAACGATCAGTAATCTAGCACTAATCTTC
TGAACACTCAAGAATGTAAGCCCATCATCACACACATCGTTTTTGCGCTT
CACTTTTTATCAGTGCGGTCAGAACTTCAGCCTGAGTCAGGCCATCTTCA
TGACACATTTGCATGAGCATGGCCTTATACTTTGGTTCAAGAAATACTTT
TACTTCCTTGAACGAAGCTCTTTTACGGGCCACTGATAATCTTTGTTTCT
CTGCATCAGAAAGCGGATTCCCCTTTCTGTATGCTCGTTTTGCGCCAGAT
GAGGAAGTCACTGCATTTTCTGTCTGCGACATCTCGCCTCCTCAATACTT
AAACAGGGATCGTTTCGCAGAGGATACTACAGTTTTTTGAAATCAGCGAC
TTGAGAATTGTGACGAAGATCCGGGATTACCCTGTTATCCC.

This sequence may otherwise be referred to as an anti-IncF cassette.

In some embodiments the recombinant vector comprises a nucleic acid sequence which is adapted to inhibit replication of IncI plasmids. Thus, the recombinant vector may comprise an anti-IncI (anti-I) cassette. The anti-IncI cassette may comprise a replication control region that is 100% conserved in IncI plasmids from many strains of Salmonella enterica and Escherichia coli, for example coordinate 3236 to 3368 from pSTM2 (Accession KF390378.2). A suitable anti-I cassette is described by Freire-Martin et al., (2016) J Med Microbiol. 65, 611-618.

An anti-IncI cassette may comprise the sequence:

(SEQ ID NO. 6)
TGTTCCGGAAGCCATAAAAGGAAAACCCCCACTATCTTTCTTACGAACTT
GGCGGAACGACGAAAGATAGTGGGGGCCTCACAGAATACGGGTAAAGTAT
AATGAAACCGTACCAGAGATTCAACCCTGTGCA.

In some embodiments the recombinant vector comprises a nucleic acid sequence which is adapted to inhibit replication of IncK plasmids. The recombinant vector may thus comprise an anti-IncK (anti-K) cassette. The anti-IncK cassette may comprise a replication control region from an archetypal IncK plasmid, such as R387 from E. coli. For example, the IncK cassette may comprise coordinates 475 to 1014 of R387 (Accession M93063.1), as shown in below.

Anti-IncK cassette sequence:

(SEQ ID NO 7)
ccatggccataaggcattcaggacgtatggcagaaacgacggcagtttg
ccggtgccggaaggctgaaaaaagtttcagaaggccataaaggaaaacc
cccactatctttcctcgaactttggcgggctcgtgaaagatagtagggg
cgttcacagaatacgggataagtatatatgaaaccgtaccagagattca
accctgtgcagtgtataaatacacggcacaatcgctccgccataagcga
cagcttgtggcaggtctgaagaatacttcatataacgcagtacactgga
gtcagttagcacccgaagagcagatccgtttctgggaagactatgaagc
gggaagggcgaccactttcctggttgaaccggaaaggaagcgcacgaag
cgccgtcgcggtgagcactccaccaaacccaaatgcgaaaatccgtcct
ggtatcgtccggagcgctataaggcgctgagcgggcagctcgggcacgc
ctacaaccgtctggtgaaaaaggacccggtgaccggcgagcagagcctg
cg.

In some embodiments, the inhibitory effect of the recombinant vector on the target plasmid may be due to binding sites for a replication protein that plays both a negative and a positive role, and which allows ‘handcuffing’ and therefore blocking of the replication origin or other essential parts of the replicon of the target plasmid. For example, in the case of an exemplary anti-IncF cassette, the incC region from the FIA replicon contains five binding sites (iterons) with a consensus sequence TGAGGGT^{T}/_{A}^{G}/_{A}TTTGTCACAG (SEQ ID NO 8) which are necessary for replication inhibition.

Nucleic acid sequences which enable the recombinant vector to compete with and/or inhibit replication of the target plasmid may be referred to as “anti-replication segments”.

It is known that a significant problem involved with curing or displacing target plasmids is host cell death, which occurs when the target plasmid encodes a so-called Post-Segregational Killing (PSK) system. This happens because the plasmid leaves behind either (i) protein, which becomes active as a toxin after loss of the plasmid; or (ii) mRNA, which is translated to produce a toxin. Action of the toxin, which is either lethal or bacteriostatic to the host, is normally prevented by either: (i) regulators, such as antisense RNA, which control translation of the mRNA that is left behind; or (ii) antidote proteins, which counteract the toxic effects of the toxin. The regulators, the antidote proteins, and the antisense RNA are all encoded by the plasmid, and are unstable, and therefore decay once the plasmid that encodes them is no longer present in the host. Therefore, the result is death or cessation of growth of the host cells from which the plasmid has been displaced.

In previous work (described in WO2007/001682), a recombinant displacement vector was designed which incorporated part of a PSK system encoded by the plasmid to be displaced. This was found to be very effective at curing endogenous plasmids encoding a PSK system from a host cell.

Thus, in some embodiments the recombinant vector is adapted to neutralize the toxic effects of a post-segregational killing (PSK) system of a target plasmid.

The term “post-segregational killing (PSK) system” as used herein refers to any of the known mechanisms adopted by plasmids that prevent plasmid-free segregants from surviving. For example, any of the terms known in the art such as: killer system; killing-anti-killing; post-segregational killing; toxin-antitoxin; poison-antidote; plasmid addiction system; or programmed cell death, are all terms that are used by the skilled technician to describe a suitable mechanism used to selectively kill a host cell if it does not contain a copy of the plasmid, and are therefore analogous to the term “PSK system”. The term “PSK system” also encompasses other systems that have effectively the same properties, namely restriction modification systems and bacteriocin production/immunity systems. It will be appreciated that the PSK system usually comes into play after cell division.

The recombinant vector is adapted to neutralise the toxic effects of the target plasmid's PSK system to avoid host cell death, and this may be achieved in several ways. In some embodiments, the recombinant vector is capable of genetically complementing the antidote part of the PSK system on the plasmid being displaced from the host cell. In some embodiments, the recombinant vector is capable of genetically complementing at least one, or each, of the antidote-encoding genes of the PSK systems of the target plasmid. By the term “genetically complement”, we mean that the recombinant vector encodes at least a region of the same PSK system as on the target plasmid so that once the plasmid has been displaced from the cell, the genes “lost” are compensated for or retained in the cell due to being encoded on the recombinant vector.

In some embodiments, the recombinant vector comprises at least a region of an antidote-encoding gene or a functional variant thereof of the PSK system encoded by the target plasmid. The recombinant vector may comprise a nucleic acid sequence having substantially the same sequence as that of the antidote-encoding gene of the PSK system of the target plasmid. It will be appreciated that although similar in function, the sequence of the genes making up various PSK systems will vary between organisms, and some examples are provided herein. Nevertheless, the skilled technician would know how to determine or at least predict the sequence of the antidote-encoding gene or genes of the PSK system encoded by the target plasmid. It is also envisaged that the recombinant vector may comprise a functional variant of the antidote-encoding gene of the PSK system of the target plasmid.

By the term “functional variant of the PSK system”, or “functional variant of an antidote-encoding gene”, we mean that the sequence of the recombinant vector (or the amino acid sequence encoded thereby) which enables the vector to neutralize the toxic effects of the PSK system has at least 60%, at least 70%, at least 80%, at least 90%, at least 95%, at least 97%, at least 98% or at least 99% sequence identity with the amino acid/and/or nucleic acid sequence of the PSK system, or at least the antidote-encoding gene(s) on the target plasmid.

The skilled technician will appreciate how to calculate the percentage identity between two amino acid/polynucleotide/polypeptide sequences. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

The recombinant vector may be adapted to neutralise the toxic effects of the target plasmid's PSK system in several ways. For example, the recombinant vector may encode a regulator protein, which is adapted to modulate expression of a toxin gene of the plasmid's PSK system into mRNA. The regulator protein may be adapted to minimise or substantially prevent expression of the plasmid's toxin gene.

Alternatively, or additionally, the recombinant vector may encode antisense RNA, which is adapted to bind to and prevent the toxic action of any toxic mRNA (generally but not necessarily translation to produce a toxic protein), which may be produced by the target plasmid.

Alternatively, or additionally, the recombinant vector may encode an antidote protein, which is adapted to bind to and prevent the toxic action of any toxic protein which may be produced by the target plasmid.

Alternatively, or additionally, the recombinant vector may encode a DNA modification enzyme, which is adapted to prevent the toxic action of any restriction endonuclease, which may be produced by the target plasmid.

Alternatively, or additionally, the recombinant vector may encode an immunity protein, which protein is adapted to prevent the toxic action of any secreted toxin protein (generically called a bacteriocin), which may be produced by the target plasmid.

In some embodiments the nucleic acid molecule comprises an antidote gene from at least one PSK system independently selected from: flmA/flmB (hok/sok) (e.g. from F plasmid, accession number AP001918, coordinates 62824-62927); srnB/srnC (e.g. from p1658/97, accession no. AF550679, or from pB171, accession no. AB024946) (antisense RNA systems); letA/letB (ccdA/ccdB) (e.g. from pO157, accession no. AB01158, coordinates 28000-29500); and pemI/pemK (e.g. from p1658/97 and pB171) (a toxin/anti-toxin system).

In some embodiments, the recombinant vector comprises both the sok gene and the letA gene, which may be operatively linked together.

Nucleic acid sequences which encode an antisense RNA, and antidote genes encoding an antidote protein, a DNA modification enzyme and/or an immunity protein, may be referred to as “anti-addiction” segments or regions, since they confer on the host cell the ability to lose the target plasmid without cell death. The anti-addition and anti-replication segments can be combined in a nucleic acid “cassette” which can conveniently be inserted into a suitable plasmid to provide the recombinant vector of the invention.

Thus, in some embodiments, the first nucleic acid sequence comprises an anti-replication segment, which inhibits and/or competes with replication of the target plasmid, and an anti-addition segment, which neutralises the toxic effects of the target plasmid's PSK system.

In some embodiments, the recombinant vector further comprises a control region that regulates transcription of the vector backbone. The control region may include genes such as ccr/par genes.

In some embodiments, elements of the vector such as the origin of replication and/or replicon(s), the gene(s) encoding an antidote to a PSK system, and/or a gene encoding an inhibitor molecule are operatively linked together.

It will be appreciated that the recombinant vector may comprise elements that induce expression of the genes it encodes and, optionally, replication of the vector. Such elements may include one or more promoters and regulator units associated with gene expression and replication.

In some embodiments, the recombinant vector comprises a nucleic acid sequence encoding the formation of thin and flexible pili. An example of such thin and flexible pili is those encoded by the IncI1 plasmid R64 (accession number NC_005014.1, genes pilV to pilI and nucleotides 104790 to 117081). Other sequences encoding suitable pili will be available to the skilled person. Such pili may facilitate conjugative transfer of the recombinant vector and thus aid transfer rates between host cells in the gut.

In some embodiments, the recombinant vector comprises a self-destruct mechanism. For example, the iscel gene encoding a homing endonuclease from Saccharomyces cerevisiae under control of an inducible promoter, such as the araBAD promoter, plus regulatory gene araC along with the 18-base pair nucleotide recognition sequence for the endonuclease encoded can linearise the plasmid DNA in vivo, preventing normal replication and thus loss (Herring et al., GENE Volume: 311, 153-163). Other self-destruct mechanisms are described in the literature and will be known to the skilled person. Such mechanisms may help to ensure that the vectors do not persist once curing has occurred.

In some embodiments, the recombinant vector comprises one or more genes encoding a selectable marker. For example, the vector may comprise a gene encoding resistance to an antibiotic, such as tetracycline (designated Tc^{R}), kanamycin (Kn^{R}) or ampicillin (Ap^{R}).

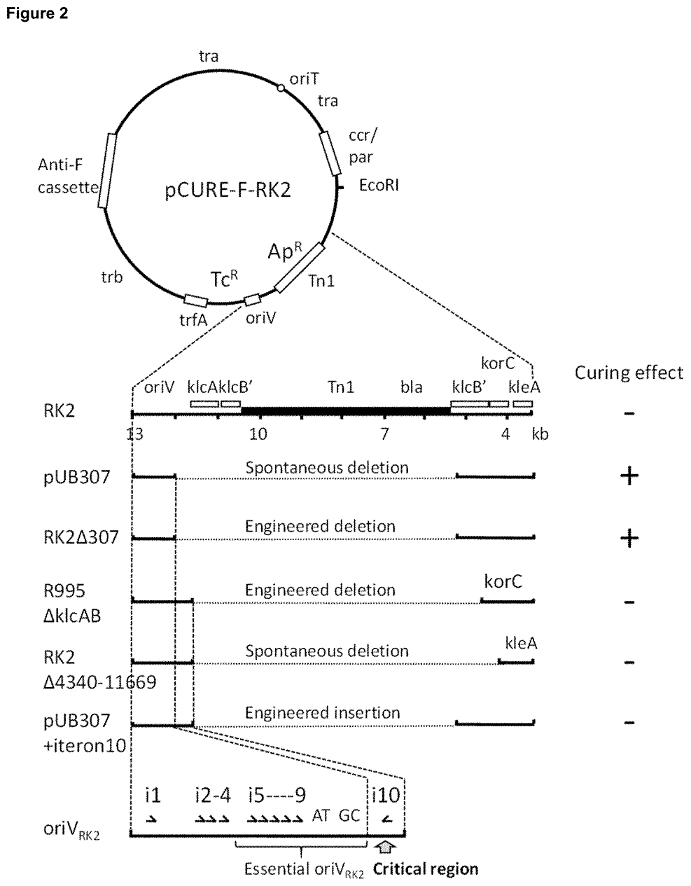

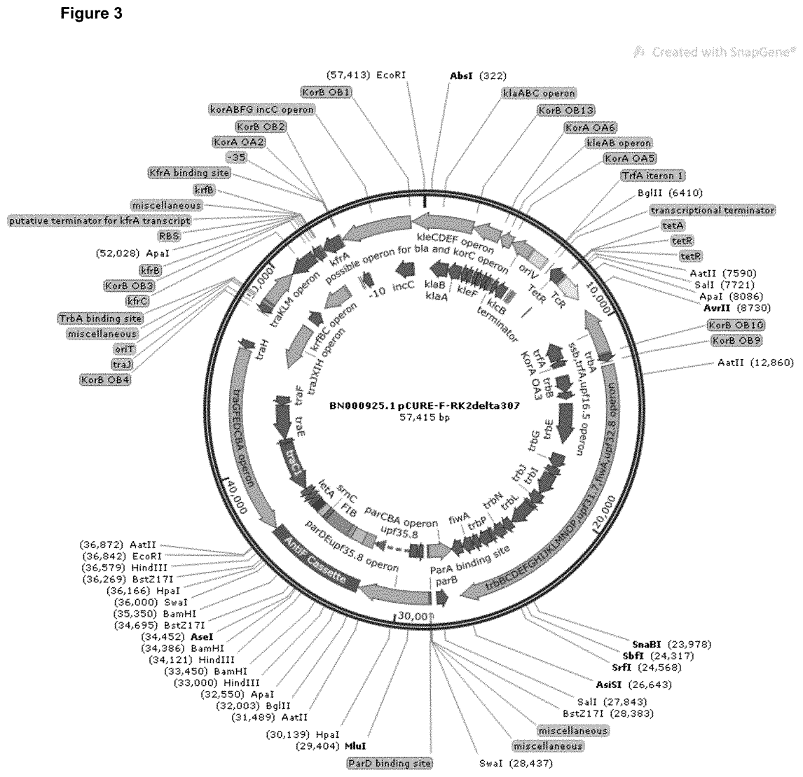

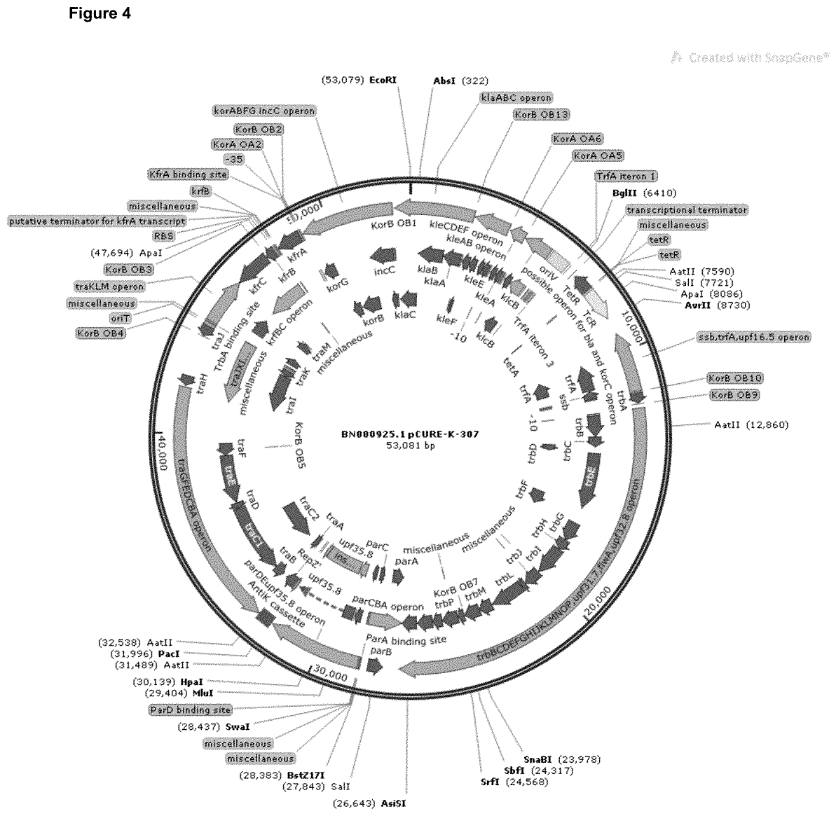

In some embodiments, the recombinant vector is pCURE-F-307 or pCURE-F-RK2Δ307 (FIG. 2; FIG. 3), or a variant which is at least 70%, 80%, 85%, 90%, 95%, 96%, 97%, 98%, 99% identical in sequence thereto.

pCURE-F-307 can be prepared using the methods described herein, starting from a naturally occurring derivative of RP1 (which is identical to RK2), pUB307 (Grinsted, J. et al., Plasmid 1, 34-37, 1977).

pCURE-F-RK2Δ307 was derived by first deleting from RK2 the bases that are missing from RP1 in pUB307 and then inserting the anti-F cassette in the same way as for pCURE-F-307. pCURE-F-307 and pCURE-F-RK2Δ307 are thus expected to be substantially the same in sequence. The sequence of pCURE-F-RK2Δ307 is shown as SEQ ID NO. 9.

In some embodiments, the recombinant vector is pCURE-EK499-307, or a variant which is at least 70%, 80%, 85%, 90%, 95%, 96%, 97%, 98%, 99% identical in sequence thereto. pCURE-EK99-307 can be constructed using the methods described herein. It differs from pCURE-F-307 by having three copies of the FIIA copAB region, the third being derived from plasmid pEK499 so that it is able to produce the variant copA anti-sense RNA that can block translation of the rep mRNA that is essential to produce the FIIA Replication protein. The same FIIA copA copB region as already derived from pO157 and pKDSC50 was amplified by PCR from pEK499 to place an EcoRI site at one end and an MfeI site at the other. These two restriction enzymes generate the same sticky end so that when the pEK499 copAB segment is inserted into the single EcoRI site in the antiF cassette an EcoRI is left at just one end, allowing similar insertions to be repeated if further segments need to be added in future.

The recombinant vectors described above comprise the anti-IncF cassette from pCURE2, which was designed to displace Inc-F plasmids. This cassette includes loci that inhibit replication (repFIA, incC, repFIB, repFIIA, copAB; from pO157; repFIIA, copAB from pKDSC50,) and that neutralize addition systems (flmB, sok; letA, ccdA; pemI and srnC, sok), as described by Hale et al., (2010) Biotechniques, 48(3), 223-228. Advantageously, these vectors may be used to displace an F-like plasmid from a host cell.

In some embodiments, the recombinant vector is pCURE-K-307 or pCURE-K-RK2Δ307, or a variant which is at least 70%, 80%, 85%, 90%, 95%, 96%, 97%, 98%, 99% identical in sequence thereto. The nucleic acid sequence of pCURE-K-307 is shown as SEQ ID No. 10.

The vector pCURE-K-307 comprises an anti-K cassette, which was designed to displace IncK plasmids. This cassette comprises the IncK replication control region of archetypal plasmid R387. Thus, pCURE-K-307 may be used to displace an IncK plasmid from a host cell.

According to a further aspect, there is provided a method of displacing a target plasmid from a host cell, the method comprising introducing a conjugative recombinant vector into the host cell, wherein the recombinant vector is derived from an IncP parent plasmid and comprises an IncP replicon that is modified to have an elevated copy number relative to that of the parent plasmid.

In some embodiments, the IncP replicon comprises a deletion near to the origin of replication (oriV). This deletion results in the elevated copy number relative to the parent plasmid. In some embodiments the deletion is within 0.5 kb of oriV.

In some embodiments, the method comprises introducing the system of the first aspect of the invention, or the conjugative recombinant vector of the second aspect of the invention, into the host cell. In some embodiments, the conjugative recombinant vector is derived (i.e. created from) from pUB307.

Since the recombinant vector of the invention is self-transmissible, it can be introduced into the host cell by conjugative transfer from a donor bacterium. The method of displacing a target plasmid from a host cell may therefore comprise mixing donor cells containing the recombinant vector with recipient host cells containing the target plasmid. The donor and recipient cells may be in liquid culture. Techniques for conjugative transfer of plasmids (e.g. by filter mating) will be well-known to those in the art. Transconjugants (i.e. host cells carrying the recombinant vector) can then be selected for by plating onto agar containing an antibiotic which is selective for the recombinant vector.

Alternatively, the recombinant vector may be introduced into a host cell by transformation, using standard techniques.

In some embodiments in which TrfA and/or KorB is expressed in trans, the first and/or the second nucleic acid sequence (which may be present in one or more further recombinant vectors) comprising the trfA and/or the korB gene may be introduced into the host cell via transformation.

In a further aspect, the present invention provides a cell containing the system or the recombinant vector of the invention. The cell may be a prokaryotic (e.g. bacterial) cell or a eukaryotic cell, as described hereinabove.

The cell may be a bacterial donor cell for conjugative transfer of the system or vector into a host cell. The donor cell may be a gram positive or a gram negative bacterial cell, such as E. coli. The donor cell is also referred to herein as a “donor strain”.

According to another aspect, there is provided a composition comprising the system, the recombinant vector or the cell of the invention.

The composition may be formulated as a liquid (e.g. a suspension, solution or dispersion), a gel, a lotion, a cream or a tablet. The composition may further comprise a buffer. The buffer may be suitable for stably storing the system, the recombinant vector or the cell, for example by freezing.

In some embodiments, the composition is a probiotic, which may be in the form of a food (e.g. yoghurt or cheese) or a beverage. A probiotic composition may contain the system or the recombinant vector within a bacterial strain (i.e. a donor strain) which is established as being safe for animal or human use, such as E. coli Nissle1917.

The probiotic composition may contain other ingredients, e.g. other bacterial strains, which are believed to be beneficial. Health benefits have been associated with specific probiotic strains of the following genera: Lactobacillus, Bifidobacterium, Saccharomyces, Enterococcus, Streptococcus, Pediococcus, Leuconostoc, Bacillus, and Escherichia coli.

According to a further aspect, there is provided a kit comprising:

(i) a recombinant conjugative vector according to the second aspect of the invention, a system according to the first aspect, or a cell containing the system or the recombinant vector of the invention; and
(ii) an instruction manual.

In some embodiments, the kit may further comprise a first nucleic acid molecule encoding TrfA. In some embodiments, the kit may further comprise a second nucleic acid molecule encoding KorB.

In some embodiments, the kit may additionally comprise a bacterial (donor) strain for enabling conjugative transfer of the recombinant vector to a host cell containing a target plasmid. The bacterial strain may be E. coli (e.g. E. coli Nissle1917).

In a further aspect, there is provided a method of treatment of a bacterial infection in a subject, the method comprising administering to the subject the recombinant vector, the system, the cell (or donor strain) or the composition of the present invention.

The subject is typically an animal, e.g. a mammal, especially a human.

Treatment may comprise administering a therapeutically effective amount of the recombinant vector, the system, the cell or the composition, to a subject in need thereof. It would be within the capability of the skilled person to work out an appropriate dosage to deliver a therapeutically effective amount. The optimum dosage may vary depending on factors such as the copy number of the target plasmid and the recombinant vector, the type of host cell, and the type of donor cell (i.e. the cell containing the recombinant vector or system of the invention).

The recombinant vector, the system, the cell or the composition may be administered by any suitable means, such as oral ingestion, topical application, subcutaneous administration or administration via a mucous membrane.

The vector, system, cell or composition (the “agents” of the invention) may be used alone, or in combination with other therapeutic agents. The agents may be administered simultaneously, or sequentially. It will be appreciated that in a combination therapy, each component need not be administered in the same manner. For example, one therapy may be administered by oral administration and another by intravenous administration.

In some embodiments the method further comprises administering an antibiotic to the subject. This may be useful for selecting for cells which carry the recombinant vector or system of the invention, wherein the vector or system comprises a gene which encodes resistance to said antibiotic. The antibiotic may be administered simultaneously with the recombinant vector, the system, the cell or the composition of the invention, and/or subsequently. For example, following administration of a composition comprising a donor strain containing the recombinant vector, the subject may be administered an antibiotic which selects for the vector over a period of a few days.

In yet a further aspect, there is provided the recombinant vector, system, cell or composition as described herein for use as a medicament.

In another aspect, there is provided the recombinant vector, system, cell or composition as described herein for use in the treatment of a bacterial infection.

### DETAILED DESCRIPTION

Embodiments of the invention will now be described by way of example and with reference to the accompanying figures in which:

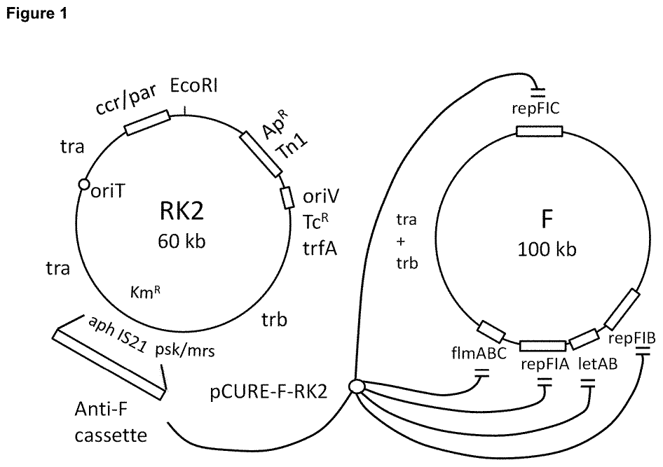

FIG. 1 is a map of RK2 showing the region replaced by the anti-F cassette and showing the targets of the anti-F functions. The other functions marked are: oriV, the vegetative replication origin; oriT, the transfer origin; tra and trb regions encoding proteins for DNA processing and mating bridge formation during transfer; trfA, encoding the Replication protein that activates oriV; ccr/par, the central control region that regulates transcription of RK2 backbone genes and also encodes active partitioning functions; and psk/mrs, encoding addiction and multimer resolution functions. Mobile elements Tn1 and IS21 are associated with ampicillin and kanamycin resistance genes. Recombineering was used to replace aphA and IS21 in RK2 with the anti-F cassette that blocks the repFIA, repFIB and repFIC replicons and neutralises the effect of the flmABC and letAB addiction loci;

FIG. 2 shows the conjugative IncP-1 derivatives constructed and their effectiveness as vehicles for plasmid curing. The anti-F cassette when inserted into RK2 itself caused limited target plasmid loss (Table 3) whereas when inserted into pUB307 it was found to cause very efficient target plasmid loss. Introducing the pUB307 deletion (bases 5464/5466 to 12045/12047) into RK2 by recombineering also potentiated curing while the deletions ΔklcAB and ΔklcABC (bases 4340-116699) did not. Reintroducing the region with iteron 10 (bases 11749 to 12048) by recombineering into pUB307 destroyed the potentiation;

FIG. 3 is a map of the plasmid pCURE-F-RK2Δ307;

FIG. 4 is a map of the plasmid pCURE-K-307;

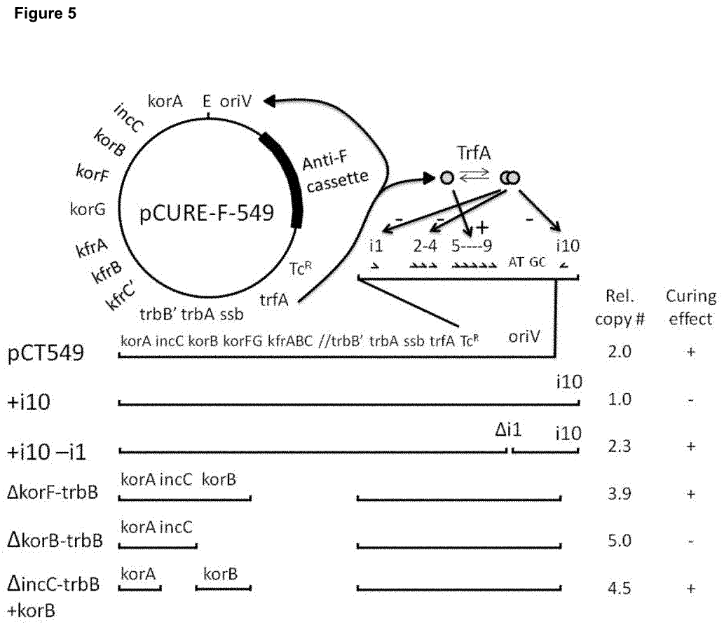

FIG. 5 shows Mini-RK2 plasmids with the anti-F cassette and ability to displace F′prolac. Plasmid pCT549 consists of two segments from RK2: oriV to trbB′ and korA to kfrC′ (apostrophe indicates a truncated gene). TrfA acts at oriV: monomers act positively through iterons 5-9 and dimers act negatively through all the iterons. Due to its construction oriV of pCT549 excluded iteron 10 (i10) and with anti-F cassette efficient curing was seen. A derivative with iterons 1 to 10 gave a lower relative copy number and efficient curing is lost. Deletion of i1 restores efficient curing and copy number. Deleting up to korB had no effect but deleting past it destroys curing. Reinserting korB restores efficient curing;

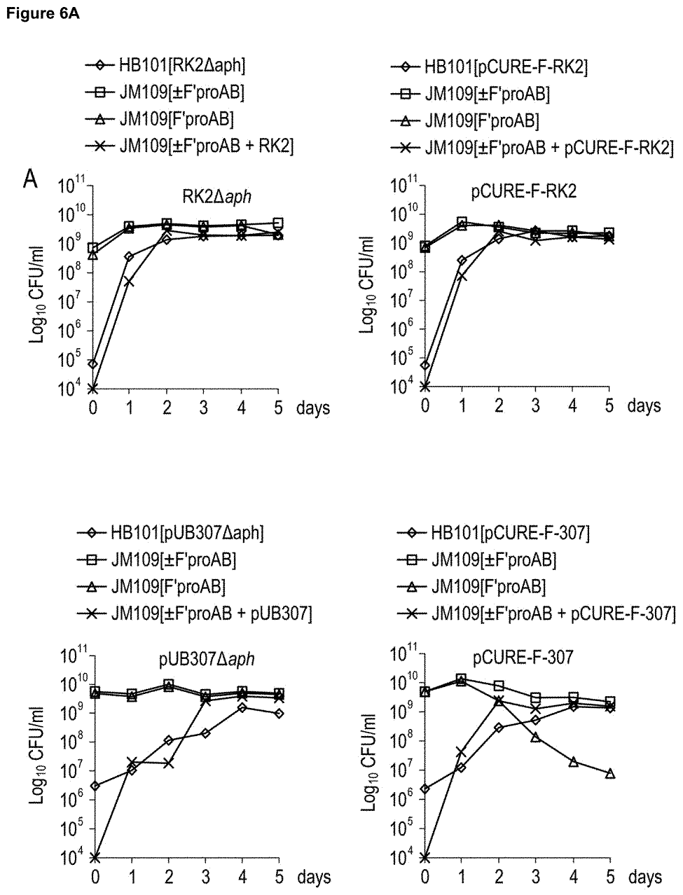

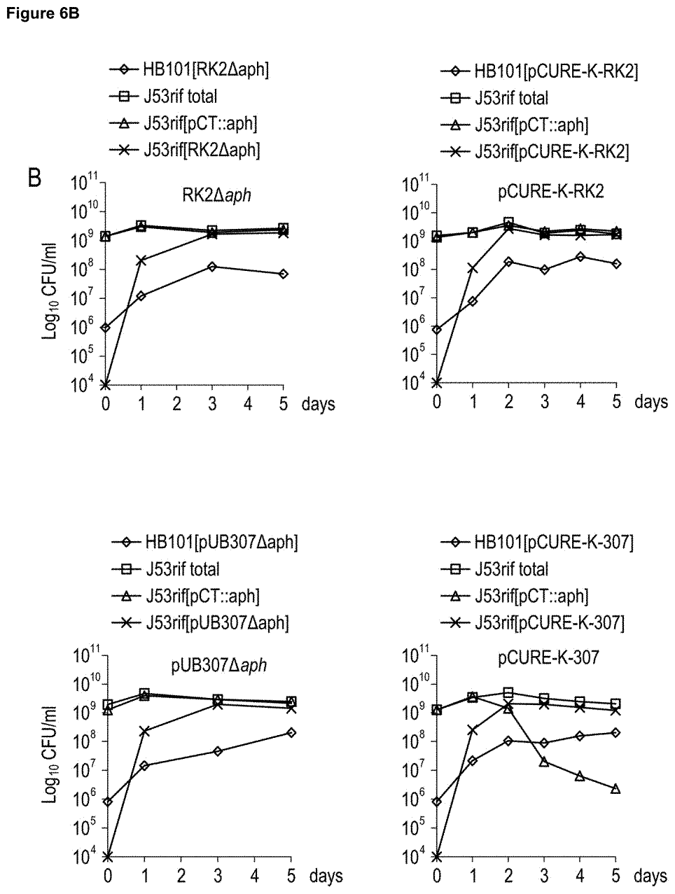

FIG. 6 shows graphs showing the results of an unselected invasion assay to monitor displacement of target plasmids by pCURE plasmids or negative controls in bacteria introduced at a donor:recipient ratio of 1:1000. Data presented is the mean of triplicate cultures. A steep decline in target plasmid was observed with pCURE-F-307 and pCURE-K-307 which was reproducible and highly significant. FIG. 6A. Donor bacteria (HB101 with pCURE-F plasmids or negative controls) were monitored by streptomycin resistance, while target bacteria (JM109 initially with F′prolac) were monitored by nalidixic acid resistance. Presence of F′prolac was monitored on M9 Minimal medium without proline. B. Donor bacteria (HB101 with pCURE-K plasmids or negative controls) were monitored by streptomycin resistance, while target bacteria (J53rif initially with pCT::aph) were monitored by rifampicin resistance. Presence of pCT::aph was monitored by kanamycin resistance. Spread of pCT::aph into donor bacteria was detected by selection of kanamycin and streptomycin resistance;

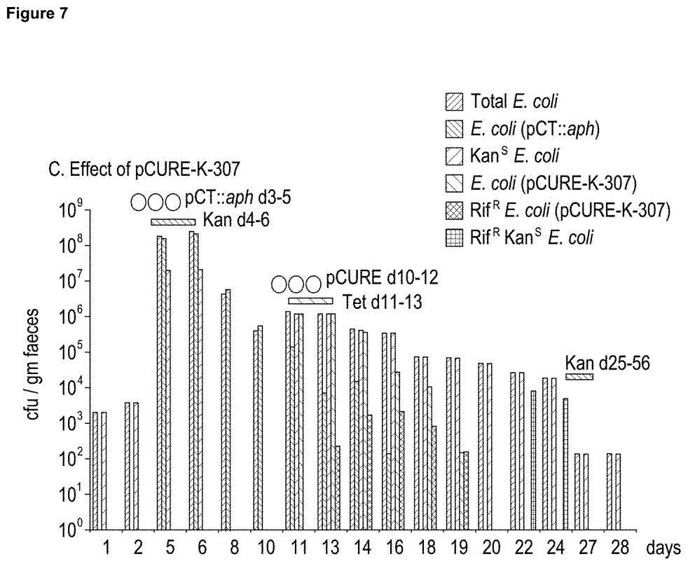

FIG. 7 shows the result of experiments to determine the effect of recombinant vectors on antibiotic resistance plasmids in the mouse gut. Rif^{R} mouse-derived E. coli AL1 carrying pCT::aph was fed to mice on days 3-5 and kanamycin on days 4-6 before monitoring plasmid carriage until day 28. Mice received E. coli Nissle 1917 carrying Tet^{R} curing plasmid, pCURE-K-307 days 10-12 and tetracycline days 11-13 resulting in the appearance of Tet^{R} E. coli including Rif^{R} Tet^{R} indicating transfer to target bacteria. pCURE-K-307 had disappeared by day 20 but about 10% of the E. coli were Rif^{R} indicating displacement of pCT::aph rather than loss of the strain. Kanamycin was given days 25-26 but no Kan^{R} bacteria re-appeared and PCR screening of faeces samples at the end of the experiment proved negative. Carriage of pCT::aph was determined by Kan^{R} while carriage of pCURE-K-307 was determined by Tet^{R};

FIG. 8 is a sequence alignment of oriV region sequences for plasmids RK2, R751 and pDS3 (accession number JX469834.1), with the iterons identified. Numbers above the line indicate the coordinate of the starting nucleotide for that segment.

### EXAMPLES

The well-studied IncP-1 Birmingham plasmid RK2 (essentially identical to RP1 and RP4) was selected as a starting point (FIG. 1) since IncP plasmids, which were originally identified as responsible for the spread and maintenance of carbenicillin resistance in skin and gut bacteria in a Burns Unit in 1969, has a transfer system that can promote transfer between both Gram negative and positive bacteria. Moreover, RK2 also has a copy number in the region of 3-7 per chromosome which is higher than many of the large conjugative plasmids that would be prime targets for the strategy so it was thought that the cloned segments should be active in blocking replication of their target replicons. The course of implementing this strategy described below revealed exciting and unexpected new information about IncP-1 plasmid biology as well as helping define a strategy for building a successful conjugative curing plasmid.

### Materials and Methods

### Bacterial Strains, Plasmids and Growth Conditions

The bacterial strains used were Escherichia coli DH5α, C600, MV10NaIR, JM109, HB101, Nissle1917, AL1 (this study, Rif^{R} mutant of mouse E. coli strain). Plasmids used in or constructed as part of this study are listed in Table 1.

TABLE 1
Plasmids used and constructed during this study

| Plasmid | Properties | Reference |
|---|---|---|
| RK2 | IncP-1α; Amp^{R} Kan^{R} Tet^{R} | Ingram, L. C., |
| | | et al. (1973) |
| | | Antimicrobial |
| | | Agents and |
| | | Chemotherapy |
| | | 3, 279-288. |
| pUB307 | IncP-1α; Kan^{R} Tet^{R}; spontaneous deletion that lost Tn1 | Grinsted, J. et |
| | | al. (1973) |
| | | Plasmid 1, 34- |
| | | 37. |
| pR9242 | IncP-1α (R995); Kan^{R} Tet^{R}; site-directed deletion of KlcA | |
| | Tn1 | |
| F′ proAB | RepFIA RepFIB RepFIC/FIIA Tra⁻ in JM109 | Bhattacharyya, |
| | | A. et al. (2001) |
| | | J. Mol. Biol. |
| | | 310, 51-67. |
| pMS208A8.2 | pMB1 replicon, korC_{RK2}:Amp^{R} | Thomas, C. M. |
| | | et al. (1998) |
| | | Nucl. Acids. |
| | | Res. 15, 5345- |
| | | 5359. |
| pCURE2 | pMB1 replicon, oriTRK2, sacB, anti-IncF; Ap^{R} Kan^{R} | Hale, L. et al. |
| | | (2010) |
| | | BioTechniques. |
| | | 48 (3), 223-228 |
| pEK499 | IncF Tra⁻ Amp^{R} Sm^{R} Su^{R} Cam^{R} Tet^{R} Cp^{R} Tp^{R} | Woodford N. et |
| | | al. (2009) |
| | | Antimicrob |
| | | Agents |
| | | Chemother, 53, |
| | | 4472-82. |
| pCT::aph | IncK Tra⁺ Kan^{R} | Cottell, J. L. et |
| | | al. (2011) |
| | | Emerg. Infect. |
| | | Dis. 17, 645- |
| | | 652. |
| pMEL1 | From pACYC184:p15A replicon; Cam^{R}, Tet^{R}, sacB | This study |
| pMILL1 | pMEL1 with 500 bp arms from RK2 (coordinates 38,075- | This study |
| | 38,573: arm 1; 39,555-40,056: arm 2) to insert antiF | |
| | cassette | |
| pMILL2 | pMILL1 with anti-IncF cassette inserted as a Bg/II-AatII | This study |
| | fragment | |
| pLAZ1 | pMILL1 without EcoRI site in cat, Cam^{R} | This study |
| pLAZ2 | pLAZ1 with anti-F cassette inserted as a Bg/II-AatII | This study |
| | fragment; Cam^{R} | |
| pLAZ2.1 | pLAZ2 with pEK499 copAB inserted in EcoRI site of | This study |
| | anti-F cassette; Cam^{R} | |
| pLAZSOE1 | pMEL1 with SOEd arms for pUB307 deletion. Cloned | This study |
| | into HindIII and SaII sites; Cam^{R} | |
| pLAZSOE4 | pMEL1 with 2x 500 bp from RK2 to introduce the iteron | This study |
| | 10 region back into pUB307 | |
| pSLK1 | pMILL1 with anti-IncK cassette inserted as an Ncol to | This study |
| | BamHI fragment | |
| RK2Δaph | IncP-1α, Amp^{R}, Tet^{R}, Δaph from RK2 mediated by | This study |
| | pMILL1 | |
| RK2Δ307 | IncP-1α, Kan^{R} Tet^{R}, RK2 with site directed deletion | This study |
| | identical to that in pUB307 made using pLAZSOE1 | |
| RK2ΔklcA-korC | IncP-1α, Km^{R} Tc^{R} spontaneous deletⁿ between klcAp & | This study |
| | kleAp | |
| pUB307Δaph | IncP-1α, Tet^{R}, Δaph from pUB307 mediated by pMILL1 | This study |
| pUB307::iteron10 | pUB307 with iteron 10 after recombineering with | This study |
| | pLAZSOE4 | |
| pCURE-F-RK2 | IncP-1α, Amp^{R}, Tet^{R}, antiF cassette inserted via pMILL2 | This study |
| | into RK2 | |
| pCURE-F-307 | IncP-1a, Tc^{R}, antiF cassette inserted via pMILL2 into | This study |
| | pUB307 | |
| pCURE-F-9242 | IncP-1α, Tet^{R}, antiF cassette inserted via pMILL2 into | This study |
| | pR9242 | |
| pCURE-F- RK2 | IncP-1α, Tet^{R}, RK2ΔklcA-korC-derivative (Δ4340- | This study |
| ΔklcA-korC | 11669) with antiF cassette inserted by recombineering | |
| | using pMILL2 | |
| pCURE-F- | IncP-1α, Tet^{R}, pUB307::iteron10 with antiF cassette | This study |
| 307::i10 | inserted by recombineering using pMILL2 | |
| pCURE-FEK499- | IncP-1α, TetR, antiF cassette with extra copA copB | This study |
| 307 | segment from pEK499 inserted via pLAZ2.1 into | |
| | pUB307 | |
| pCURE-K-RK2 | IncP-1α, Amp^{R}, Tet^{R}, antiK cassette inserted via pSLK1 | This study |
| | into RK2 | |
| pCURE-K-307 | lIncP-1α, Tet^{R}, antiK cassette inserted via pSLK1 into | This study |
| | pUB307 | |
| pCT549 | Mini IncP-1α, Kan^{R}Tet^{R}, | Thomas, C. M. |
| | korA⁺incC⁺korB⁺korF⁺korG⁺kfrA⁺B⁺C⁻ | et al. (1984) |
| | | EMBO J 3, 57- |
| | | 63. |
| pCURE-F-549 | Mini IncP-1α, Tet^{R}, antiF, | This study, FIG. |
| | korA⁺incC⁺korB⁺korF⁺korG⁺kfrA⁺B⁺C⁻ | 3 |
| pCURE-F- | Mini IncP-1α, Tet^{R}, antiF, | This study, FIG. |
| 549::i10 | korA⁺incC⁺korB⁺korF⁺korG⁺kfrA⁺B⁺C⁻ | 3 |
| pCURE-F- | Mini IncP-1α, Tet^{R}, antiF, | This study, FIG. |
| 549::i10Δi1 | korA⁺incC⁺korB⁺korF⁺korG⁺kfrA⁺B⁺C⁻ | 3 |
| pCURE-F-549 | Mini IncP-1α, Tet^{R}, antiF, korA⁺incC⁺korB⁺ | This study, FIG. |
| ΔtrbB-korF | | 3 |
| pCT549ΔtrbB- | Mini IncP-1α, Tet^{R}, antiF, korA⁺incC⁺ | This study, FIG. |
| korB | | 3 |
| pCURE-F-549 | Mini IncP-1α, Tet^{R}, antiF, korA⁺inc⁻korB⁺ | This study, FIG. |
| ΔtrbB-incC::korB | | 3 |
| pRK2501 | Mini IncP-1α, Tet^{R}, Kan^{R}; korA⁺incC⁺korB⁻ | Kahn, M. et al. |
| | | (1979) Meth. |
| | | Enzymol. 68, |
| | | 268-280. |
| pRK2501::antiF | Mini lncP-1α, Tet^{R}, Km^{R}; korA⁺incC⁺korB⁻ | This study |

E. coli strains were cultured aerobically, in either L-broth/L-agar or M9 Minimal Medium, at 37° C. Final concentrations of antibiotics used were: Ampicillin (Amp), 100 μg/ml; kanamycin (Km or kan), 50 μg/ml; chloramphenicol (Cm), 50 μg/ml; nalidixic acid (nal), 25 μg/ml; and tetracycline (Tet or tet), 25 μg/ml. For the blue white screening L-agar was supplemented with X-gal (20 μg/ml) and IPTG (0.5 mM).

### DNA Analysis and Manipulation

Restriction enzymes were purchased from New England Biolabs; T4 DNA ligase and Taq DNA polymerase were from Invitrogen; Velocity proof-reading DNA polymerase was from Bioline; Q5 high fidelity Taq polymerase was from NEB. PCR amplification of DNA was achieved using the primers (AltaBioscience, University of Birmingham, UK; or Sigma Aldrich) listed in Table 2. Reactions were cycled in a SensoQuest Lab Cycler following standard procedures. PCR products were purified using the Illustra GFX™ PCR DNA and Gel Band Purification Kit (GE™ Healthcare). Small-scale plasmid DNA preparations were performed using the AccuPrep Plasmid MiniPrep DNA Extraction Kit (Bioneer) adapted from the alkaline lysis method of Bimboim and Doly. DNA sequencing reactions were prepared and run on an ABI 3730 DNA analyser (Functional Genomics Facility, University of Birmingham, U.K.) following the chain termination method.

TABLE 2
Primers designed and used during this study

| | | | |
|---|---|---|---|
| | | Template, | |
| Primer | Base sequence (5′-3′ where not indicated)^{a} | comments | SEQ ID No. |

Amplification of RK2 arms to allow integration in place of the aph gene

| | | | |
|---|---|---|---|
| RK2 Arm 1F | AGGCGTCGACCAAAGGGTTCGCAGACTG | RK2 | SEQ ID No. 11 |
| | GGG | | |
| RK2 Arm 1R | GACGTCGCTAACAGATCTTCCTTAATTAAG | | SEQ ID No. 12 |
| | GCATCCCTGACAGACAACGC | | |
| RK2 Arm 2F | TTAATTAAGGAAGATCTGTTAGCGACGTCC | | SEQ ID No. 13 |
| | AGGGAGGCGTTCAGGACGAC | | |
| RK2 Arm 2R | CCGCAAGCTTCACAGCCGGGGCATCTTTG | | SEQ ID No. 14 |
| | AG | | |

Amplification of anti-IncF and anti-IncK functions

| | | | |
|---|---|---|---|
| Anti-IncFF | GTCGACGTCCCCTGTTATCCCTACCCGG | pCURE2 | SEQ ID No. 15 |
| Anti-IncFR | GCGAGATCTAGGGTAATCCCGGATCTTCG | | SEQ ID No. 16 |
| Anti-IncK | AGCCATGGCCATAAGGCATTCAGGA | R387 | SEQ ID No. 17 |
| Anti-IncK | GTGGATCCGCAGGCTCTGCTCG | | SEQ ID No. 18 |
| AL IncK F | ATGGTGACAAAGAGAGTGCAAC | pCT::aph | SEQ ID No. 19 |
| AL IncK R | TTACAGCCCTTCGGCGATG | | SEQ ID No. 20 |

Amplification of copAB region from pEK499

| | | | |
|---|---|---|---|
| 499 copAB F | GTCCAATTGGTCGACCGTCACAATTCTCAA | pEK499 | SEQ ID No. 21 |
| | GTCGC | | |
| 499 copAB R | GTCCAATTGCTCGAGGTCACACCATCCTG | pEK499 | SEQ ID No. 22 |
| | CACTTAC | | |

Creation of deletion from pUB307 in RK2

| | | | |
|---|---|---|---|
| 307Δ Arm1F | CGCGTCGACTAGCCGTAGCACGACTCGAT | RK2 | SEQ ID No. 23 |
| | G | | |
| 307Δ Arm1R | CAATTACGTCTCCCATTACGACCATGCGC | RK2 | SEQ ID No. 24 |
| 307Δ Arm2F | CGTAATGGGAGACGTAATTGAGCATTTCC | RK2 | SEQ ID No. 25 |
| | AGGC | | |
| 307Δ Arm2R | CGGAAGCTTGGCGGACGTTGACACTTGA | RK2 | SEQ ID No. 26 |

Reinsertion of region with iteron 10 into pUB307

| | | | |
|---|---|---|---|
| +i10 Arm1F | CGCGTCGACCCGCTAGATCGCAAAGGAT | RK2 | SEQ ID No. 27 |
| +i10 Arm1R | GAATCGGGTATCCCATTACGACCATGCGC | RK2 | SEQ ID No. 28 |
| +i10 Arm2F | CGTAATGGGATACCCGATTCTGCGGTTAC | RK2 | SEQ ID No. 29 |
| | A | | |
| +i10 Arm2R | TATGCCGCCGGACGTAATTGAGCATTTCC | RK2 | SEQ ID No. 30 |
| | AGG | | |
| Mutate | ATGCTCATCCGGAGTTCCGTATGGCAATG | pACYC184 | SEQ ID No. 31 |
| EcoRI in | AAAGACG | | |
| pACYC184 | CGTCTTTCATTGCCATACGGAACTCCGGAT | pACYC184 | SEQ ID No. 32 |
| | GAGCAT | | |

Manipulation of mini-RK2 plasmid pCT549

| | | | |
|---|---|---|---|
| oriV + i10F | ATCGAATTCCGGCCGTACCCGATTC | | SEQ ID No. 33 |
| oriV + i1R | GAGATAGATCTAGCGTGGACTCAAG | | SEQ ID No. 34 |
| oriV - i10F | CATGAATTCGTTTAGAGCGAGCCAGGAAA | | SEQ ID No. 35 |
| | G | | |
| oriV - i1R | TGAAGATCTACCGCAGGGAAATTCTCGTC | | SEQ ID No. 36 |
| BglII-PacI- | 5′ | | SEQ ID No.s 37 |
| HindIII linker | AGCTTACGTTAATTAAATGTACGACGTCCT | | and 38 |
| | AA 3′ (SEQ ID NO 37) | | |
| | 3′ATGCAATTAATTTACATGCTGCAGGATTC | | |
| | TAG 5′ (SEQ ID NO 38) | | |
| MfeIPacloriV | ATCCAATTGGATTTAATTAACCGGCCGTAC | With i10 | SEQ ID No. 39 |
| | CCGATTC | | |
| oriVEcoRIXbaI | ATTCTAGATACGAATTCTACCTCAAGGCTC | With i10 | SEQ ID No. 40 |
| | TCGCGAATG | | |
| MfeIPacloriV | ATCCAATTGGATTTAATTAAGTTTAGAGCG | Without i10 | SEQ ID No. 41 |
| | AGCCAGGAAAG | | |
| oriVEcoRIXbaI | ATTCTAGATACGAATTCTACCTCAAGGCTC | Without i10 | SEQ ID No. 42 |
| | TCGCGAATG | | |
| ΔkorF-trbBa^{b} | ATGTCTAGAACTGTCAAAGCGCACCCG | | SEQ ID No. 43 |
| ΔkorF-trbBc^{b} | ATGTCTAGACGCTGTCTTTGGGGATCAGC | | SEQ ID No. 44 |
| ΔkorB-trbBc^{b} | ATGTCTAGACCGCAGTCATTGGGAAATCT | | SEQ ID No. 45 |
| | C | | |
| ΔincC-trbBc^{b} | ATGTCTAGACCGTGACCAAAGTTTTCATCG | | SEQ ID No. 46 |
| korB F | AGTGCATGCGAAGATGGAGATTTCCCAAT | | SEQ ID No. 47 |
| | G | | |

^{a}Restriction sites are shown underlined.
^{b}″c″ indicates clockwise on the standard map of RK2 i.e. running with the coordinates in the RK2 Genbank file. ″a″ indicates anticlockwise.

### Comparison of Plasmid Copy Number

A minimum of triplicate selective overnight (16 h) cultures of E. coli carrying the query plasmids plus 2 kb pACYC194 derivative pDS3 as internal standard in question grown in LB with shaking at 200 rpm and 37° C. were harvested and then plasmid DNA extracted as described above. Plasmid DNA was digested with an enzyme that would cut just once, to linearise the DNA and make ethidium bromide binding uniform. Band intensities were determined with QuantityOne software from Biorad and normalised against the pDS3 band. Colonies from serial dilution of the cultures were replica plated to determine % plasmid carriage.

### Conjugative Transfer

Following overnight growth, 100 μl of donor was mixed with 1 ml of E. coli MV10 NaIR recipient and filtered onto a 0.45 μm sterile Millipore filter. Filters were placed on L agar plates which were incubated at 37° C. for 6 hours. Cells from the filter were resuspended in 1 ml of 0.85% sterile saline solution each and serially diluted before spreading on selective agar and incubation at 37° C.

### Recombinational Engineering (Recombineering) of Conjugative IncP-1 Plasmid Genomes

For insertions, deletions or replacements primers as listed in Table 2 were used to amplify approximately 500 bp arms on either side of the point or region to be changed. The arms were joined by designing the internal primers to have complementarity, sometimes incorporating new restriction sites, to allow joining together by SOEing (Splicing by Overlap Extension) PCR. To do this the initial PCR products were purified, mixed and then extended for three cycles before adding the external primers for the remaining cycles. The product was routinely cloned between HindIII and SalI sites of a pACYC184 derivative pMEL1 that has the sacB gene (allowing counter-selection with sucrose) inserted between the XbaI and HindIII sites. To incorporate the antiF cassette this was then inserted into pMILL1 between the homology arms (using BglII and AatII sites designed in the inner primers) to give pMILL2.

The recombineering plasmid was introduced into E. coli C600 that already carries the target plasmid, selecting with Cam and an antibiotic appropriate for the target plasmid (routinely Tet). Conjugative transfer to MV10naIR was then carried out once again selecting both markers and Nal—the pMEL1-derived plasmid should not transfer unless it has undergone recombination with the conjugative plasmid. Individual colonies were restreaked to purify and single colonies from these plates used to inoculate liquid cultures were grown overnight without Cm. Selection of resolution products was then achieved either by spreading on L-agar with sucrose (5% w/v) or by isolating plasmid DNA and cutting with XbaI that cuts in pMEL1 but not in the IncP-1 backbone to linearise the unwanted plasmids which will not transform bacteria.

pCURE-F-307 was constructed in the same way as pCURE-F-RK2 but starting from pUB307 which is like RK2 but has the deletion referred to above that runs from position 5464/5466 to 12045/12047 (there are three bases at the junction that could come from either side of the deletion) relative to the IncP-1 genome sequence.

pCURE-F-RK2Δ307 was constructed by first re-creating the pUB307 deletion starting from RK2 and then inserting the antiF cassette into this derivative as for pCURE-F-RK2 and pCURE-F-307. The 307 deletion was created in RK2 using pLAZSOE1 (Table 1 above) which contained the spliced homology arms defining the deletion (see Table 2 under the heading “Creation of deletion from pUB307 in RK2”).

pCURE-K-RK2Δ307 and/or pCURE-K-307 were constructed in the same way starting from RK2Δ307 and pUB307 using recombineering plasmid pSLK1 (Table 1) which is pMILL1 (containing the homology arms to insert cassettes in place of the IncP aph gene) with the anti-K cassette inserted instead of the anti-F cassette.

pCURE-FEK499-307 was constructed using the recombineering plasmid pLAZ2.1 which is essentially pLAZ2 with the IncFIIA copAB segment amplified from pEK499 (using the primers listed in Table 2 under the title “Amplification of copAB region from pEK499”) inserted at the EcoRI site in the antiF cassette. However, because there is an EcoRI site in the cat gene (conferring chloramphenicol resistance) of pLAZ2 we went back to pMILL1 and did site directed mutagenesis to destroy the EcoRI site without altering the polypeptide encoded. We then inserted the anti-F cassette as for pMILL2, giving pLAZ2 and then cut this with EcoRI to insert the copAB segment from pEK499. This plasmid with an expanded anti-F cassette was then used to insert the cassette into pUB307 as indicated above.

### Testing Curing Efficiency

For transfer by conjugation, overnight liquid cultures of E. coli C600 carrying the pCURE plasmid to be tested, or an appropriate control, were washed to remove any selective antibiotics, then mixed 1:1 and 1:10 with a strain carrying the target plasmid and a standard filter mating carried out at 37° C. for 1 h. The bacteria from the membrane were then resuspended in 1 ml saline and serially diluted before plating on selective agar to determine the total number of transconjugants and transconjugants still carrying the target plasmid. Displacement of F′prolac was determined both by spreading on M9 medium supplemented appropriately with and without proline to determine % loss of Pro⁺ phenotype and by growth on L-agar with IPTG and X-gal when the target strain additionally carried pUC18 to determine the Lac⁺ phenotype. A similar method was used when introducing the curing plasmid by transformation except that the target bacteria were made competent by standard CaCl₂) treatment and transformation was done with purified pCURE DNA.

For the unselected invasion assay donor bacteria were mixed with target strain to give approximately 10⁶ donors and 10⁹ recipient bacteria before spreading 100 μl on a nitrocellulose filter (25 mm diameter, 0.45 μm pore size, EMD-Millipore, Darmstad, Germany) on an L-agar plate. After 24 h incubation at 37° C. the bacteria were resuspended in 2 ml saline, mixed thoroughly and both serially diluted to profile transfer and curing and 100 μl spread on a fresh nylon membrane.

### Manipulation of Mini-RK2 Plasmids

To make the pCT549 derivatives was not easy because simple insertion of EcoRI-BglII oriV fragments or BglII-PacI fragments into this plasmid proved difficult. To insert EcoRI-BglII oriV fragments without the antiF cassette the oriV segment was generated by PCR, joined to pGEM-T Easy and sequenced. The pGEMT-derivative and pCT549 were then cut with BglII and ligated before transformation into E. coli C2110, which being DNA poll deficient cannot replicate pGEM-T, selecting cointegrants. Plasmid DNA from transformants was checked for the relative orientation of the joined segments and the one chosen that could be cut with EcoRI and recircularised by ligation to replace the old oriV with the new one.

To allow the antiF cassette to be inserted beside oriV, new primers were designed putting XbaI plus EcoRI sites downstream of oriV and MfeI plus PacI sites where BglII is normally. After cloning in pGEM-T Easy and checking sequence the MfeI-XbaI oriV fragment was ligated with the pACYC184 derivative pLAZ2 cut with EcoRI and XbaI. In pLAZ2 EcoRI defines the end of the antiF cassette and the XbaI site is on the same side. The MfeI/EcoRI ends can join but do not regenerate either site. The other end of the antiF cassette in pLAZ2 is defined by a BglII site so this construction generates a BglII-antiF-PacI-oriV-EcoRI-XbaI segment and this was inserted into pCT549 by the trick described above involving BglII cutting, ligation and C2110. The antiF cassette was similarly inserted into mini-RK2 plasmid, pRK2501, that already lacked korB.

To remove parts of the korA-incC-korB-korF-korG-kfrABC region and remnants of trbB near trfA, inverse PCR was carried out on pCT549+antiF cassette with primers incorporating an XbaI site since XbaI does not cut the RK2 backbone or the antiF cassette. Long range PCR was carried out with Q5 high fidelity Taq polymerase using their recommended primer design and conditions removing korF-trbB, korB-trbB and incC-trbB. The product was purified, cut with XbaI and recircularised and transformed into DH5α. To remove incC but not korB the korB orf was amplified and it and the ΔkorF-trbB plasmid were ligated after cutting with XbaI (cuts upstream of trbBp) and SphI which cuts in incC.

### Mouse Experiments

All animal care procedures and experiments were approved by the Animal Ethics Committee of Western Sydney Local Health District (protocol 4276.08.17) in accordance with the ‘Australian Code of Practice for the Care and Use of Animals for Scientific Purposes’ and carried out essentially as described previously. Five week old female BALB/c mice (Animal Resource Centre; Perth, WA, Australia) were housed in groups of three in open-lid M1 polypropylene cages (Able Scientific, Australia) on a 12 h light/dark cycle, with food and water available ad libitum (Biological Services Facility, Westmead Institute for Medical Research). Each different treatment involved groups of 3 mice. Mice were acclimatised (d−6 to d0) before experiments, followed by run-in (d1-d3) in the experimental room to establish the baseline. Mice were fasted for 6 h before being given access to sucrose water and then normal food was continuously available. Bacterial cultures carrying a plasmid were resuspended in sucrose water (8%, w/v) to an OD600 of ˜0.4±0.05 and fed to mice on specified days and/or antibiotics (10-50 mg L-1) as appropriate. On the specified days each mouse was briefly transferred into a separate plastic box for weighing and to collect fresh faeces. Faeces (100 μg per mouse) were suspended in 1 ml phosphate buffered saline (PBS), dilutions plated on CHROMagar with appropriate antibiotics and colonies counted after incubation 0/N at 37° C. Periodically 100 colonies were picked onto further plates to determine accurate proportions of resistance phenotypes. PCR to detect the IncK plasmid replicon in Rif^{S} E. coli was done using primers AL_IncK_F and AL_IncK_R (Table 2). Mouse faeces solutions (100 mg in 1 mL saline) were diluted 1:100 times in water and 3 μl used as template in 50 μl reaction. E. coli carrying pCT::aph was used as positive control. At the end of the experiment PCR was carried out to determine whether any pCT::aph plasmid DNA could be detected if that was not evident by direct plating. Mice were euthanised by an overdose of CO₂ immediately after completion of experiments.

The groups of mice were as follows. Group 1 (control group): received normal food and drink plus sucrose water when other groups received it but without bacteria or antibiotics. Group 2 (antibiotic control group): received normal food and drink plus sucrose water with antibiotics when groups 3 & 4 received antibiotics. Group 3 (colonisation control group): received E. coli AL1 (pCT::aph) in sucrose water for days 3-5 plus Kanamycin for days 4-6, then normal food and water for the rest of the experiment, monitoring E. coli AL1(pCT::aph) in faeces until end of experiment. Group 4 (curing experimental group A): received E. coli AL1(pCT::aph) in sucrose water for 3 days plus Kan (3 days) as group 3, then challenged with E. coli Nissle1917(pCURE-K-307) for days 10-12 and Tet for days 11-13 and monitor faeces for different sets of E. coli (endogenous coloniser, curing strains and challenger). Group 5 (curing experimental group B): received E. coli AL1 (pCT::aph) in sucrose water for 3 days plus Kan (3 days), then challenged with E. coli Nissle1917 (pCURE-K-307) for 3 days but no antibiotics and monitored faeces as above. Group 6 (curing experimental group C): received E. coli AL1 (pCT::aph) in sucrose water for 3 days plus Kan (3 days), then challenged with E. coli Nissle1917 (pCURE-K-307) every day for 8 days (d10-17) and monitored faeces as above.

### Results

### Example 1: Development of Recombinant Vectors

The previously constructed anti-IncF cassette from pCURE2, designed to displace IncF plasmids, including loci that inhibit replication (repFIA, incC; repFIB; repFIC, copAB; repFIC/repFIIA, copAB) and that neutralise addiction systems (flmB, sok; letA, ccdA; pemI and srnC, sok), was inserted into RK2 by recombination to replace the aph (Kang) gene (FIG. 1). This created pCURE-F-RK2 (FIG. 2; nomenclature indicates first that it is a plasmid carrying a curing cassette, second the plasmid group it targets, and third the plasmid the vector is based on) along with a control, RK2Δaph, deleted for the aph region but without the anti-IncF cassette. The efficiency with which pCURE-F-RK2 can displace F′prolac (chosen because loss of the F plasmid can be detected by loss of the ability to grow on minimal medium without proline and by blue/white X-gal screening when the strain also carries a plasmid such as pUC18 that complements the lacZ defect in the lac operon carried by the F′) from JM109 was tested by mixing donor and recipient bacteria to allow transfer on solid agar, selecting for acquisition of pCURE-F-RK2 followed by replica plating onto appropriately supplemented M9 medium to detect loss of F′prolac (Table 3). None of the initial transconjugant colonies were completely free of F′prolac but on further culturing and screening, curing increased to between 85 and >99% while RK2 without the anti-IncF cassette as well as RK2Δaph did not affect F′prolac stability at all (Table 3). This indicates that the anti-IncF cassette, when joined to the IncP-1 plasmid to give pCURE-F-RK2, is able to sustain the unidirectional displacement of F-like plasmids, but is much less efficient at this than when in the high copy number pCURE2 plasmid.

To demonstrate curing of resistance plasmids carrying β-lactamases we inserted the anti-F cassette into a spontaneous deletion derivative of IncP-1 plasmid RP1 (indistinguishable from RK2), pUB307, because it had lost the transposon Tn1 that includes the b/a gene conferring AP^{R}. We had previously determined the ends of this deletion and found that it runs from position 5464/5466 to 12045/12047 (there are three bases at the junction that could come from either side of the deletion) relative to the IncP-1 genome sequence, removing backbone sequences flanking Tn1 as well as the transposon itself (FIG. 2). This new plasmid (pCURE-F-307) was unexpectedly found to be much more effective than pCURE-F-RK2 in causing displacement of F′prolac from JM109 (Table 3). To check whether the potentiation of curing activity in the pUB307-derived plasmids is due to this deletion rather than to other changes elsewhere in the plasmid, we recreated the pUB307 deletion starting from RK2, using a reverse-genetics approach as described above. The same potentiation of curing was observed (Table 3; pCURE-F-RK2Δ307), confirming that the deletion in pUB307 is indeed responsible for this effect.

To determine whether this increased ability to cure is specific for F-like plasmids we inserted the IncK replication control region of archetypal plasmid R387 as an anti-IncK cassette into both RK2 and pUB307 at the same location and tested for curing of IncK plasmid pCT::aph. Once again, low efficiency curing was observed with the pCURE-K-RK2 and high efficiency with pCURE-K-307. To check whether the potentiation is specific to delivery by conjugative transfer (which involves a single-stranded DNA intermediate) we introduced the plasmids by transformation. This showed that the same potentiation was observed irrespective of how the plasmid entered the target bacteria. Since the deletion removes the start of the klcA-klcB-korC operon it may affect expression of korC that encodes a transcriptional repressor providing autogenous control of the operon. The deletion could cause either increased expression due to removal of the normal autogenous control or decreased expression due to the absence of an obvious alternative promoter. We therefore introduced a plasmid expressing korC, pMS208A8.2, into the JM109 recipient with F′prolac for the curing experiment and repeated the experiment, comparing the curing ability of pCURE-F-RK2 and pCURE-F-307, but this did not affect the curing observed (data not shown). Therefore it seems unlikely that the potentiation seen with pCURE-F-307 is due to decreased korC expression.

TABLE 3
One- and two-step curing data for key anti-F and
anti-K plasmids constructed in this study.

| | Target plasmid and % cured ^{a} | | | |
|---|---|---|---|---|
| Curing plasmid or | F′prolac initial^{b} | F′prolac after^{b} | pEK499 (IncF) | R387 (IncK) initial |
| control (ctl) | colonies | re-culturing | initial colonies | colonies |
| RK2 (vector ctl) | <1 | <1 | <1 | <1 |
| RK2Δaph (vector ctl) | <1 | <1 | <1 | <1 |
| pCURE-F-RK2 | <1 | >85 | <1 | ND^{c} |
| pUB307 (vector ctl) | <1 | <1 | <1 | <1 |
| RK2Δ307 (vector ctl) | <1 | <1 | ND | ND |
| pCURE-F-307 | >99 | ND | <1 | ND |
| pCURE-F-RK2Δ307 | >99 | ND | ND | ND |
| pCURE-K-RK2 | ND | ND | ND | <1 |
| pCURE-K-307 | ND | ND | ND | >99 |
| pCURE-FEK499-307 | >99 | ND | >99 | ND |

^{a} These tests were carried out multiple times on separate occasions. Comparisons were done by replica plating 100 colonies. The phenotype was generally very clear: <1 means 0/100 colonies had lost the target plasmid; >99 means 100/100 had lost the target plasmid; >85 means we saw significant curing but there were always some colonies that retained the target plasmid and 85% cured was the lowest rate observed. Blue/white screening could also be used and gave a clear cut the difference between efficient and inefficient curing as shown in FIG. 2.
^{b}Stage 1 involved screening transconjugant colonies from initial selection plates for complete loss of Pro+ phenotype. Stage 2 involved re-culturing from initial colonies into LB medium + tetracycline selective for RK2 or pCURE-F-RK2, growing O/N, plating on L-agar + tetracycline and then screening for retention of the Pro+ phenotype.
^{c}Not Done

To explore further the genetic basis for the potentiation, we used deletions that remove sub-segments of the region deleted in pUB307. Plasmid pR9242 is a deletion derivative of R995 that removes essentially all of klcA and klcB by creating an in-frame fusion of the first six codons of klcA, a 6 bp XbaI recognition site and the stop codon of klcB. When the anti-F cassette was inserted into this plasmid (R995ΔklcAB), in the same location as in pCURE-F-RK2, no potentiation was seen (FIG. 2). In attempting to delete further DNA between klcAp and oriV we isolated a spontaneous deletion that removed klcA, klcB and korC (RK2Δ4340-11669) leaving the kleA operon being expressed from the klcAp. The anti-F cassette was inserted into this deletion derivative in the same location but once again the curing efficiency was similar to that of pCU RE-F-RK2 (FIG. 2), indicating that removal of all or part of the klcA, klcB, korC operon is not the reason for the potentiation of curing.

The last segment deleted in pUB307 to be tested is immediately adjacent to oriV and contains a single repeated sequence motif called an iteron (iteron 10, FIG. 2) that should bind the Rep protein, TrfA. The role of this single iteron has not been studied but deletion of the other single iteron (iteron 1) on the opposite side of oriV has been shown to increase copy number. To test this we used homologous recombination to reinsert the short region between oriV and klcA that contains iteron 10, giving pUB307::iteron10 and then combined this with the anti-F cassette (giving pCURE-F-307::i10). A curing test showed that the potentiation had been lost (FIG. 2). Attempts to confirm the change in copy number by qPCR did not give reliable data but determination of the kanamycin resistance levels conferred by the constitutively expressed aph gene in RK2 and pUB307 showed that the presence of the region with iteron 10 reduced resistance about 2-fold, corresponding to an approximately 2-fold change in copy number.

Direct confirmation of a copy number difference associated with the presence/absence of iteron 10 was obtained by comparison of isolated plasmid DNA intensity for mini-IncP-1 plasmid pCT54930 which, as constructed originally, does not have iteron 10 (FIG. 3). We amplified the oriV region from RK2 with iteron 10 and substituted it into the pCT549 backbone. Comparison of plasmid DNA yield, with and without iteron 10, indicated the copy number difference was about 2-fold (FIG. 3). Nevertheless, the same change in plasmid curing ability was observed in these mini-IncP-1 plasmids with anti-F cassette when the region with iteron 10 was absent (FIG. 3). We also considered the possibility that the change in potency could be due to some other property encoded in the region that contains iteron 10 so with this region intact we deleted the region with iteron 1 (bases 12927 to 12990 of RK2 according to the complete IncP-1 backbone). Deletion of iteron 1 resulted in a similar rise in copy number and similar potentiation of curing to removal of the region with iteron 10 suggesting that it is the presence or absence of these lone iterons that is responsible for the change in curing activity (FIG. 3).

As part of this analysis we also used as vector an even smaller derivative of RK2, pRK2501 that does not include the korB gene from the central control region and so is partially de-repressed for trfA (the rep gene) expression and has a higher copy number (2.5 compared to pCT549 with iteron 10). To our surprise this did not support curing of the F′prolac from JM109 despite its elevated copy number, suggesting that at least one additional factor in the RK2 backbone may be necessary for the curing activity by the anti-F cassette. We deleted the major block of backbone genes in pCT549 that are not essential for replication—from the trbB promoter to the start of the korF gene (removing kfrA, kfrB and the remaining part of kfrC as well as korF and korG). This leaves just oriV, the trfA region (encoding the Rep protein TrfA) plus the central control/active partitioning region32 (encoding repressor KorA, partitioning ATPase IncC [a ParA homologue] and centromere-binding protein and global repressor KorB) and observed that this did not result in loss of potentiation of curing efficiency. Since the only major difference between this derivative and pRK2501 is the absence of a functional korB in pRK2501 it appeared that korB must be necessary for the potentiation and this was confirmed by creating a deletion of all of korB from the pCT549 backbone (FIG. 3). We therefore reinserted korB into the deletion derivative that had lost most of incC and found that this derivative had regained the ability to displace the target F plasmid efficiently (FIG. 3). This indicates that the potent curing ability of pCURE307 is not only dependent on manipulation of the oriV region but also requires an intact korB gene although a complete set of par functions is not essential (FIG. 3).

### Example 2: Effectiveness of Recombinant Vectors

A critical test of a conjugative pCURE is whether it can spread through a population and displace target plasmids in the absence of selection. Therefore E. coli HB101 (pCURE-F-307) was mixed with E. coli JM109 at a ratio of 1 donor:1000 recipients and 10⁸ bacteria of the mixture placed on a nylon filter on L-agar. After overnight growth, the bacteria on the filter were washed off with 2 ml saline before re-placing on a fresh nylon filter on L-agar as well as serially diluting to sample composition. This was repeated to give 5 cycles of growth and pCURE plasmid spread. The results showed that the pCURE plasmids spread rapidly in the absence of selection and that pCURE-F-307 was able to spread and reduce the target F plasmid in a reciprocal way, with plasmid positive bacteria falling to less than 0.1% of the target population (FIG. 6). This experiment demonstrates that the elements we have shown are required for efficient curing are effective without selection. The same test was used to demonstrate displacement of IncK plasmid pCT::aph from J53RifR bacteria (FIG. 6B). Because pCT::aph is conjugative, it can potentially spread to the donor bacteria. We observed that pCT::aph spread efficiently to the HB101 bacteria in the negative control tests where the donor bacteria carried IncP plasmids lacking the anti-F cassette but, where they carried the anti-F cassette, pCT::aph was not able to establish itself.

Since F-like plasmids are the commonest plasmid types encountered among multi-resistance plasmids in Enterobacteriaceae, it is important that our anti-F pCURE plasmids can be adapted to displace all possible targets. To demonstrate the feasibility of this, we chose pEK499 which we found not to be displaced by the anti-F cassette in pCURE2 and pCURE-F-307 (Table 3). Bioinformatic analysis revealed that this may be because the copA antisense RNA region of pEK499 shows 3 mismatches to the specificity loop in the repFIIA segment in the pCURE2 anti-F cassette so that its repFIIA replicon may not be inhibited by the anti-FIIA element of the anti-F cassette. The copAB region of pEK499 region was therefore amplified and incorporated into the anti-F cassette before it was inserted into pUB307 to give pCURE-FEK499-307. As predicted, the addition of this region allowed pEK499 displacement (Table 3). This shows that the inability of pCURE2 and pCURE-F-307 to displace pEK499 was due to the lack of activity against the FIC/FIIA replicon and shows the ease with which specificity of the anti-F cassette can be extended.

### Example 3: Mouse Studies

The final test of the system was to determine whether our conjugative pCURE could spread in an animal gut model. We chose the IncK plasmid pCT::aph as the model target for displacement as it has been shown to persist in diverse E. coli strains in different animals and humans. It also has the advantage of possessing an active conjugative transfer system, in contrast to either the F′prolac or pEK499, neither of which are Tra+, thus representing the toughest sort of in vivo challenge. pCURE-K-307 was the test curing plasmid and we performed the tests in mice. After a number of exploratory tests, we chose to establish pCT::aph (the plasmid to be displaced from the mouse gut) by isolating an E. coli strain from a mouse in our first experiment, selecting a rifampicin resistant mutant, introducing pCT::aph into this strain (designated AL1) by conjugation in the lab and then feeding the plasmid-positive strain to the target mice. The strain established itself very efficiently (up to 10⁹ cfu/g faeces) but pCT::aph spread to other resident gut bacteria was only detected when the mouse received kanamycin (to select for the aph gene on the plasmid) the day after adding the plasmid-positive strain, and for the two following days (FIG. 5). That the Kan^{R} Rif^{S} bacteria carry pCT::aph was demonstrated by PCR with IncK-specific primers. In the control group of mice with no further treatment the level of Kan^{R} E. coli stabilised and remained for the rest of the experiment (FIG. 5B). In the test group, once the level of pCT::aph was stable, we attempted to displace it by feeding the mouse with E. coli Nissle1917 strain (which is accepted as a safe probiotic for human use) carrying pCURE-K-307. The colonisation by Nissle1917(pCURE-K-307) of the mouse gut was also very good (we detected ˜10⁷ cfu/g faeces). However, efficient transfer and displacement of pCT::aph was only detected in the group of mice that received Nissle1917(pCURE-K-307) with subsequent tetracycline for 3 to 4 days (FIG. 5B). Once the Nissle1917(pCURE-K-307) was established by this period of selection, which in itself did not eliminate the target bacteria, pCURE-K-307 was observed to increase in target bacteria in vivo in the absence of selection but was then lost (FIG. 5B). That transfer occurs in the gut is clear from the fact that the proportion of the Tet^{R} bacteria that are Rif^{R} rises with time to 100%, suggesting that the donor strain disappears faster than the transconjugants. That the target plasmid had disappeared completely was shown by a period of kanamycin selection near the end of the experiment—this caused the E. coli counts to fall 100-fold but the remaining E. coli were still kanamycin sensitive. PCR with IncK-specific primers was also used to confirm the absence of pCT::aph from the gut of the treated mice. This demonstrates not only successful elimination of the target plasmid but also that the short selection with antibiotics allows some endogenous microbiota to survive.

### DISCUSSION

This research demonstrates the feasibility of constructing an effective broad host range conjugative plasmid vector system that can spread without selective pressure and can specifically displace target plasmids of different incompatibility groups. This shows that the potential to ‘re-sensitise’ resistant bacterial populations is real and this could be an important alternative strategy to combat antimicrobial resistance. We also show that displacement is effective in the mouse gut following a period of selection.

### REFERENCES

1. Hale et al., (2010) Biotechniques, 48(3), 223-228;
2. Freire-Martin et al., Curing vector for IncI1 Plasmids and its use to provide evidence for a metabolic burden of IncI1 CTX-M-1 plasmid pIFM3971 on Klebsiella pneumonia (2016) J Med Microbiol. 65, 611-618;
3. Herring et al., Gene replacement without selection: regulated suppression of amber mutations in Escherichia coli, Gene. 311, 153-163;
4. Grinsted, J., Bennett, P. M. & Richmond, M. H. A restriction enzyme map of R-plasmid RP1. Plasmid 1, 34-37, 1977;
5. Ingram, L. C., et al. (1973) Antimicrobial Agents and Chemotherapy 3, 279-288.
6. Grinsted, J. et al. (1973) Plasmid 1, 34-37;
7. Bhattacharyya, A. et al. (2001) J. Mol. Biol. 310, 51-67;
8. Thomas, C. M. et al. (1998) Nucl. Acids. Res. 15, 5345-5359;
9. Woodford N. et al. (2009) Antimicrob Agents Chemother, 53, 4472-82;
10. Cottell, J. L. et al. (2011) Emerg. Infect. Dis. 17, 645-652;
11. Thomas, C. M. et al. (1984) EMBO J 3, 57-63.;
12. Doran et al, Journal of Biological Chemistry. 273, 8447-8453;
13. Shah et al., Journal of Molecular Biology. 254, 608-622;
14. Kahn, M. et al. (1979) Meth. Enzymol. 68, 268-280.

1. A conjugative recombinant vector for displacing a target plasmid from a host cell, the vector being capable of replicating in the host cell and adapted to compete with and/or inhibit replication of the target plasmid,
wherein the vector is derived from an IncP parent plasmid comprising an IncP replicon which comprises an origin of replication (oriV), or a functional fragment or variant thereof, that is associated with a series of iterons, and
wherein the vector comprises the IncP replicon comprising a deletion in one or more of the iterons relative to the parent plasmid.

2. The recombinant vector according to claim 1, wherein the recombinant vector comprises the IncP replicon comprising a deletion in one or both terminal iterons of the series, relative to the parent plasmid.

3. The recombinant vector according to claim 1, wherein the recombinant vector comprises the IncP replicon comprising a deletion in an iteron other than iterons 5-9 (i5-i9), relative to the parent plasmid.

4. The recombinant vector according to claim 1, wherein the recombinant vector comprises an IncP replicon comprising a deletion in iteron 1 and/or iteron 10 (i10), relative to the parent plasmid.

5. The recombinant vector according to claim 1, wherein the vector further comprises a first nucleic acid sequence which encodes TrfA, or a homologue, functional fragment or variant thereof.

6. The recombinant vector according to claim 1, wherein the vector further comprises a second nucleic acid sequence which encodes KorB or a homologue, functional fragment or variant thereof.

7. The recombinant vector according to claim 1, wherein the recombinant vector has a copy number which is greater than that of the parent plasmid.

8. The recombinant vector according to claim 1, wherein the recombinant vector comprises the transfer genes oriT, tra and trb.

9. The recombinant vector according to claim 1, wherein the recombinant vector comprises a nucleic acid sequence comprising all or selected parts of an origin of replication or one or more replicons of the target plasmid, such that the recombinant vector is adapted to inhibit replication of the target plasmid.

10. The recombinant vector according to claim 1, wherein the recombinant vector comprises a nucleic acid sequence encoding an inhibitor molecule which inhibits or prevents replication of the target plasmid.

11. The recombinant vector according to claim 1, wherein the recombinant vector is adapted to neutralize the toxic effects of a post-segregational killing (PSK) system of the target plasmid.

12. The recombinant vector according to claim 1, wherein the recombinant vector comprises one or more genes encoding a selectable marker.

13. A system for displacing a target plasmid from a host cell, the system comprising:
a) a conjugative recombinant vector which is capable of replicating in the host cell, wherein the vector is derived from an IncP parent plasmid comprising an IncP replicon, the IncP replicon of the parent plasmid comprising an origin of replication (oriV), or a functional fragment or variant thereof, which is associated with a series of iterons,
wherein the vector comprises an IncP replicon comprising a deletion in one or more of the iterons relative to the parent plasmid, and
wherein the vector is adapted to compete with and/or inhibit replication of the target plasmid;
b) a first nucleic acid sequence which encodes TrfA, or a homologue, functional fragment or variant thereof; and
c) a second nucleic acid sequence which encodes KorB, or a homologue, functional fragment or variant thereof.

14. (canceled)

15. (canceled)

16. A method of displacing a target plasmid from a host cell, the method comprising introducing a conjugative recombinant vector into the host cell, wherein the recombinant vector is derived from an IncP parent plasmid and comprises an IncP replicon that is modified to have an elevated copy number relative to that of the parent plasmid.

17. The method of claim 16, wherein:
i) the recombinant vector is capable of replicating in the host cell and adapted to compete with and/or inhibit replication of the target plasmid, the IncP replicon comprises an origin of replication (oriV), or a functional fragment or variant thereof, that is associated with a series of iterons, and the IncP replicon comprises a deletion in one or more of the iterons relative to the parent plasmid, or
ii) the method comprises introducing into the host cell a system comprising:
a) the conjugative recombinant vector, wherein the recombinant vector is capable of replicating in the host cell, the IncP replicon comprises an origin of replication (oriV), or a functional fragment or variant thereof, which is associated with a series of iterons, wherein the vector vector comprises an IncP replicon comprising a deletion in one or more of the iterons relative to the parent plasmid, and wherein the vector is adapted to compete with and/or inhibit replication of the target plasmid;
b) a first nucleic acid sequence which encodes TrfA, or a homologue, functional fragment or variant thereof; and
c) a second nucleic acid sequence which encodes KorB, or a homologue, functional fragment or variant thereof.

18. The method of claim 16, wherein the target plasmid carries one or more antibiotic resistance genes.

19. The method of claim 16, wherein the host cell is a bacterial cell.

20. A method of treatment of a bacterial infection in a subject, the method comprising administering to the subject:
i) a conjugative recombinant vector for displacing a target plasmid from a host cell, the vector being capable of replicating in the host cell and adapted to compete with and/or inhibit replication of the target plasmid, wherein the vector is derived from an IncP parent plasmid comprising an IncP replicon which comprises an origin of replication (oriV), or a functional fragment or variant thereof, that is associated with a series of iterons, and wherein the vector comprises the IncP replicon comprising a deletion in one or more of the iterons relative to the parent plasmid,
ii) a system for displacing a target plasmid from a host cell, the system comprising:
a) a conjugative recombinant vector which is capable of replicating in the host cell, wherein the vector is derived from an IncP parent plasmid comprising an IncP replicon, the IncP replicon of the parent plasmid comprising an origin of replication (oriV), or a functional fragment or variant thereof, which is associated with a series of iterons, wherein the vector comprises an IncP replicon comprising a deletion in one or more of the iterons relative to the parent plasmid, and wherein the vector is adapted to compete with and/or inhibit replication of the target plasmid;
b) a first nucleic acid sequence which encodes TrfA, or a homologue, functional fragment or variant thereof; and
c) a second nucleic acid sequence which encodes KorB, or a homologue, functional fragment or variant thereof, or
iii) a cell containing the recombinant vector of i) or the system of ii).

21. (canceled)

22. The recombinant vector of claim 1, wherein the vector further comprises: a) a first nucleic acid sequence which encodes TrfA, or a homologue, functional fragment or variant thereof; and b) a second nucleic acid sequence which encodes KorB, or a homologue, functional fragment or variant thereof.
